(19) United States
(12) Patent Application Publication
(10) Pub. No.: US 2022/0323482 A1
(43) Pub. Date: Oct. 13, 2022

(54) LNP-FORMULATED MRNA THERAPEUTICS AND USE THEREOF FOR TREATING HUMAN SUBJECTS

(71) Applicant: ModernaTX, Inc., Cambridge, MA (US)

(72) Inventors: Allison AUGUST, Cambridge, MA (US); Tal ZAKS, Newton, MA (US); Stephen G. HOGE, Brookline, MA (US)

(21) Appl. No.: 17/641,469

(22) PCT Filed: Sep. 11, 2020
(86) PCT No.: PCT/US2020/050546

§ 371 Date: Mar. 9, 2022

Related U.S. Application Data
(60) Provisional application No. 62/899,095, filed on Sep. 11, 2019.

(51) Int. Cl. A61K 31/7105; A61K 9/00; A61K 9/127

(52) U.S. Cl. CPC A61K 31/7105; A61K 9/0019; A61K 9/1272

(57) ABSTRACT

The disclosure features methods of treatment comprising systemic administration of mRNA encoding a therapeutic protein and delivered by lipid nanoparticle to human subjects.

### CROSS-REFERENCE TO RELATED APPLICATIONS

This application claims the benefit of U.S. Provisional Patent Application Ser. No. 62/899, 095, filed Sep. 11, 2019. The entire contents of which is incorporated herein by reference.

### BACKGROUND

Administration of a synthetic and/or in vitro-generated mRNA that structurally resembles natural mRNA can result in the controlled production of therapeutic proteins or peptides via the endogenous and constitutively-active translation machinery (e.g. ribosomes) that exists within a patient's own cells. In recent years, the development and use of mRNA as a therapeutic agent has demonstrated potential for treatment of numerous diseases and for the development of novel approaches in regenerative medicine and vaccination (Stanton et al (2017) Messenger RNA as a Novel Therapeutic Approach. In: Garnder A. (eds) RNA Therapeutics. Topics in Medicinal Chemistry, vol 27 Springer, Cham; Sabnis et al. (2018) Mol Ther 26:1509-1519; Hassett et al. (2019) Mol Ther Nucleic Acids 15:P1-11).

Treatment of certain diseases may comprise administration of a therapeutic polypeptide that are retained within a cell (e.g., an intracellular protein) or are secreted by a cell (e.g., a secreted protein). In some cases treatment comprises administration of mRNA encoding a therapeutic polypeptide that is a naturally-occurring polypeptide, whereupon expression of the mRNA has the purpose of increasing or promoting endogenous levels and/or activity of the polypeptide. While in other cases, treatment comprises administration of mRNA encoding a polypeptide that is non-naturally-occurring and/or is modified from a naturally-occurring polypeptide, whereupon expression of the mRNA has the purpose of providing a polypeptide with novel function for treatment of a disease.

It is recognized that treatment of a given disease requires that mRNA encoding a therapeutic polypeptide be produced at levels sufficient to achieve a therapeutic effect. There exists a need to develop mRNAs encoding therapeutic proteins that when administered to human subjects achieve a desired therapeutic effect to alleviate, prevent, and/or treat disease.

### SUMMARY OF THE DISCLOSURE

It has been discovered that systemic administration of mRNA formulated in lipid nanoparticles (LNPs) can produce therapeutic proteins at therapeutically-effective levels in serum and/or tissues of human subjects. As described herein, LNP-formulated mRNA administered intravenously to human subjects is taken up by the liver and translated into the therapeutically active protein. In the case of a prophylactic antibody, it has been discovered that systemic administration of an LNP-formulated mRNA encoding antibody heavy and light chains resulted in the production and secretion of functional antibody heterodimers at therapeutically effective levels in the serum of human subjects to protect against an infectious disease.

Specifically, systemic administration of two mRNAs encoding the heavy and light chains of an anti-chikungunya antibody formulated in an LNP resulted in therapeutically effective antibody levels in naïve human subjects, exceeding the level expected to be protective against chikungunya infection (>1 μg/mL), following a single dose of LNP-formulated mRNA. Surprisingly, intravenous administration of a single dose of LNP-formulated mRNA provided therapeutically effective levels of antibody above protective levels for at least 16 weeks in the human subjects, and resulted in circulating neutralizing antibody activity against chikungunya virus replication, demonstrating that the LNP-formulated mRNA resulted in the production of a fully functional therapeutic protein in vivo. Moreover, it was discovered that intravenous administration of a second dose of the LNP-formulated mRNA resulted in a serum concentration of the encoded antibody that was nearly 2-fold higher that that achieved with the first dose, demonstrating a capability to augment or increase antibody levels with repeat dosing of the LNP-formulated mRNA.

Administration of LNP-formulated mRNA for in vivo expression of an encoded antibody (e.g., a prophylactic antibody) in a human subject provides numerous advantages over administering antibody-based therapeutics, including reduced manufacturing challenges and development costs. As described herein, it has been shown that systemic administration of an LNP-formulated mRNA encoding an antibody resulted in therapeutic levels of the antibody produced in serum in the subject (e.g., a therapeutic level sufficient to neutralize an infectious agent). It has also been demonstrated that a therapeutic level of antibody was maintained in serum of a human subject for a duration necessary to achieve a therapeutic outcome (e.g., a duration longer than a period of exposure to an infectious agent), and provided a tolerable safety profile. As described herein, systemic administration of a single dose or a repeat dose of an LNP-formulated mRNA of the disclosure to human subjects, produced a therapeutic level of an encoded protein in serum (e.g., a therapeutic level of antibody sufficient to provide protection against an infectious agent, e.g., a therapeutic level having a Cmax greater than 1 μg/mL). Surprisingly, the therapeutic level of antibody was maintained in serum for an extended duration of time (e.g., greater than 16 weeks), and provided a tolerable safety profile throughout the extended duration of time (e.g., absence of serious or untreatable adverse events).

Accordingly, the present disclosure provides methods for treating and/or delaying progression of a disease or disorder in human subjects in a prophylactic or therapeutic application that would benefit from expression of a therapeutically effective level of a therapeutic protein encoded by an mRNA. For example, a disease or disorder that would benefit from therapeutically-effective levels of a therapeutic protein expressed by LNP-formulated mRNA in serum and/or a target tissue (e.g., a malignancy, an autoimmune disease, an infectious disease, or a metabolic disease). In some embodiments, the encoded therapeutic protein is an intracellular protein that performs a desired function within a cell (e.g., enzyme, a membrane-bound receptor, an antigen, a transcription factor). In some embodiments, the encoded therapeutic protein is a soluble, secreted protein (e.g., an antibody, an enzyme or recruitment factor).

In some embodiments, the disclosure provides methods for systemic administration of a dose of LNP-formulated mRNA encoding a therapeutic protein, optionally at a duration and/or frequency effective to achieve and/or maintain a therapeutically-effective level of the therapeutic polypeptide in serum and/or tissue of the subject to thereby treat and/or delay progression of a disease or disorder. In some embodiments, the dose of LNP-formulated mRNA is effective to maintain a therapeutically-effective level of the therapeutic protein for a duration, e.g., 1, 2, 3, 4, 5, 6, 7, 8, 9, 10, 11, 12, 13, 14, 15, 16, 17, 18, 19, 20, 24, 36, 48 weeks.

In some embodiments, the disclosure provides methods for systemic administration of a first dose of LNP-formulated mRNA encoding a therapeutic protein, optionally at a duration and/or frequency effective to achieve and/or maintain a therapeutically-effective level of the therapeutic polypeptide in serum and/or tissue of the subject, followed by administration of a second or subsequent dose of the LNP-formulated mRNA, optionally at a duration and/or frequency effect to achieve and/or maintain a therapeutically-effective level of the therapeutic polypeptide in serum and/or tissue of the subject. In some embodiments, the first and/or second dose of LNP-formulated mRNA is effective to maintain a therapeutically-effective level of the therapeutic protein fora duration, e.g., 1, 2, 3, 4, 5, 6, 7, 8, 9, 10, 11, 12, 13, 14, 15, 16, 17, 18, 19, 20, 24, 36, 48 weeks.

In some embodiments, the present disclosure relates to systemic administration of LNP-formulated mRNA encoding the heavy and light chains of an anti-chikungunya antibody to provide therapeutically effective levels of antibody to treat chikungunya fever and/or protect human subjects against infection with chikungunya virus (CHIKV). The CHIKV is a mosquito-born virus that poses a significant public health problem in tropical and subtropical regions. There are no vaccines approved to prevent CHIKV infection or disease, and effective mosquito control is challenging. Thus, there exists an unmet need for safe and effective therapies.

Accordingly, in some aspects, the disclosure relates to treatment of chikungunya fever and/or prevention of CHIKV infection by systemic administration of LNP-formulated mRNAs encoding the heavy and light chains of an antibody targeting CHIKV (anti-CHIKV). The disclosure is based, at least in part, on the discovery that intravenous administration of two mRNAs encoding the heavy and light chains of an anti-CHIKV antibody formulated in an LNP results in translation and assembly of a functional anti-CHIKV antibody in vivo. Indeed, administration was found to provide neutralizing antibody titers of the anti-CHIKV antibody following a single intravenous dose in both animal and human subjects. Moreover, when administered in human subjects, serum levels of the anti-CHIKV antibody exceeded a minimum target concentration expected to have a therapeutic effect, and remained elevated for an extended period following mRNA administration without significant toxicity. Without being bound by theory, the presence of serum levels of the anti-CHIKV antibody exceeding a minimum serum concentration target allows passive immunization of naïve human subjects to treat and/or prevent the onset of chikungunya fever disease symptoms.

Accordingly, in some aspects, the disclosure provides a method of producing a therapeutic level of an antibody in a human subject in vivo, the method comprising systemically administering to the subject an LNP-formulated mRNA, the mRNA encoding the antibody, in a dose effective to produce a Cmax of at least 1 μg/mL of the antibody in serum of the subject.

In some aspects, the disclosure provides a method of producing a therapeutic level of an antibody in a human subject in vivo, the method comprises administering (e.g., by intravenous injection or intravenous infusion) to the subject an LNP-formulated mRNA, the mRNA encoding the antibody, in a dose effective to produce a Cmax of at least 1 μg/mL of the antibody in serum of the subject.

In any of the foregoing or related aspects, the dose is effective to produce a Cmax of at least 2 μg/mL, at least 6 μg/mL, or at least 10 μg/mL. In some aspects, the dose is between 0.1-0.6 mg/kg. In some aspects, the dose is 0.1, 0.3, 0.45, or 0.6 mg/kg. In some aspects, the antibody has a half-life of at least 50-100 days.

In any of the foregoing or related aspects, the method comprises systemically administering a second dose of the LNP-formulated mRNA within about 1 week, about 2 weeks, about 3 weeks, or about 4 weeks following the first dose. In some aspects, the method comprises administering (e.g., by intravenous injection or intravenous infusion) a second dose of the LNP-formulated mRNA within about 1 week, about 2 weeks, about 3 weeks, or about 4 weeks following the first dose. In some aspects, the Cmax following the second dose is equal to or greater than the Cmax following the first dose. In some aspects, the Cmax following the second dose is at least 1.1-fold, 1.4-fold, or 1.8-fold greater than the Cmax following the first dose.

In some aspects, the disclosure provides a method of producing a therapeutic level of an antibody in a human subject in vivo, the method comprising systemically administering to the subject an LNP-formulated mRNA, the mRNA encoding the antibody, in a first dose effective to produce a Cmax of at least 1 μg/mL of the antibody in serum of the subject, and at least one second dose effective to produce a Cmax equal to or greater than a Cmax following the first dose.

In some aspects, the disclosure provides a method of producing a therapeutic level of an antibody in a human subject in vivo, the method comprising administering (e.g., by intravenous injection or intravenous infusion) to the subject an LNP-formulated mRNA, the mRNA encoding the antibody, in a first dose effective to produce a Cmax of at least 1 μg/mL of the antibody in serum of the subject, and at least one second dose effective to produce a Cmax equal to or greater than a Cmax following the first dose.

In any of the foregoing or related aspects, the first dose effective to produce a Cmax of at least 2 μg/mL, at least 6 μg/mL, or at least 10 μg/mL. In some aspects, the Cmax following the second dose is at least 1.1-fold, 1.4-fold, or 1.8-fold greater than the Cmax following the first dose. In some aspects, the first and second dose of LNP-formulated mRNA is between 0.1-0.6 mg/kg. In some aspects, the second dose is administered once weekly, once every 2 weeks, once every 3 weeks, or once every 4 weeks. In some aspects, the second dose is administered within one week, within 2 weeks, within 3 weeks, or within 4 weeks.

In some aspects, the disclosure provides a method of producing a therapeutic level of an antibody in a human subject in vivo, the method comprising systemically administering to the subject a repeat dose of LNP-formulated mRNA, the mRNA encoding the antibody, effective to achieve a therapeutic level having a Cmax of at least 1 μg/mL of the antibody in serum of the subject. In some aspects, the repeat dose comprises a dose of LNP-formulated mRNA systemically administered once weekly, once every 2 weeks, once every 3 weeks, or once every 4 weeks. In some aspects, the repeat dose comprises a first dose of between 0.1-0.6 mg/kg. In some aspects, the first dose is 0.1, 0.3, 0.45, or 0.6 mg/kg. In some aspects, the repeat dose comprises a second dose of LNP-formulated mRNA between 0.1-0.6 mg/kg. In some aspects, the second dose is 0.1, 0.3, 0.45, or 0.6 mg/kg. In some aspects, the Cmax following the first dose is at least 2 μg/mL, at least 6 μg/mL, or at least 10 μg/mL. In some aspects, the therapeutically-effective level has a Cmax following the second dose that is equal to or greater than a Cmax following the first dose. In some aspects, the Cmax following the second dose is at least 1.1-fold, 1.4-fold, or 1.8-fold greater than the Cmax following the first dose. In some aspects, the antibody has a half-life of at least 50-100 days.

In any of the foregoing or related aspects, the therapeutic level of the antibody in serum is maintained for a duration following systemic administration. In some aspects, the duration is at least 12 weeks, at least 13 weeks, at least 14 weeks, at least 15 weeks, at least 16 weeks, at least 18 weeks, at least 19 weeks, at least 20 weeks, at least 21 weeks, at least 22 weeks, at least 23 weeks, or at least 24 weeks.

In any of the foregoing or related aspects, the disclosure provides methods of systemically administering a dose of LNP-formulated mRNA encoding an antibody to human subjects, wherein the antibody comprises an antibody heavy chain (HC) and an antibody light chain (LC), and wherein the HC and LC are encoded on separate mRNAs. In some aspects, the mRNAs encoding the HC and LC are co-formulated in the same LNP, optionally at a 2:1 (HC:LC) w/w ratio. In some aspects, the mRNAs encoding the HC and LC are co-formulated in the same LNP, optionally at a 1:2 (HC:LC) w/w ratio. In some aspects, the mRNAs encoding the HC and LC are co-formulated in the same LNP, optionally ranging between at a 2:1 (HC:LC) to a 1:2 (HC:LC) w/w ratio. In some aspects, wherein when the LNP-formulated mRNA is systemically administered, the HC and LC are expressed from separate mRNAs and combined to form the antibody.

In any of the foregoing or related aspects, the disclosure provides methods of systemically administering a dose of LNP-formulated mRNA encoding an antibody to human subjects, wherein the antibody comprises an engineered single chain antibody, and wherein the HC and LC are encoded by a single mRNA.

In some aspects, the disclosure provides methods of systemically administering a dose of LNP-formulated mRNA encoding an antibody to a human subject, wherein the dose is effective to produce a therapeutic level of an antibody in the human subject in vivo. In some aspects, the antibody is a prophylactic antibody, optionally for use in protecting the subject against an infectious disease, optionally wherein the subject is naïve for or uninfected by the infectious disease. In some aspects, the therapeutic level of the prophylactic antibody in serum is sufficient to neutralize a target infectious agent.

In any of the foregoing or related aspects, the LNP comprises an ionizable amino lipid, a phospholipid, a cholesterol lipid or cholesterol-derivative lipid, a PEG-lipid or conjugated lipid. In some aspects, the ionizable amino lipid is Compound 1. In some aspects, the PEG-lipid is Compound 2. In some aspects, the LNP comprises 20-60 mol % ionizable amino lipid and 0.5-15 mol % PEG lipid. In some aspects, the LNP comprises 20-60 mol % ionizable amino lipid, 5-25% DSPC, 25-55% cholesterol, and 0.5-15 mol % PEG lipid. In some aspects, the LNP-formulated mRNA is systemically administered via intravenous infusion or intravenous injection. In some aspects, the LNP and mRNA are formulated in the same vial. In some aspects, the LNP and mRNA are formulated in separate vials. In some aspects, the LNP and mRNA are combined prior to being systemically administered. In some aspects, the LNP-formulated mRNA is systemically administered via intravenous infusion for a duration of 30 minutes to 4 hours.

In some aspects, the disclosure provides a method of producing a therapeutic level of an antibody in a human subject in vivo, the method comprising systemically administering to the subject an LNP-formulated mRNA, the mRNA encoding the antibody, in a dose effective to produce a Cmax of at least 1 μg/mL of the antibody in serum of the subject, wherein the LNP comprises Compound 1, a phospholipid, a cholesterol lipid or cholesterol-derivative lipid, and Compound 2. In some aspects, the LNP comprises 20-60 mol % Compound 1 and 0.5-15 mol % Compound 2. In some aspects, the LNP comprises 20-60 mol % Compound 1, 5-25% DSPC, 25-55% cholesterol, and 0.5-15 mol % Compound 2. In some aspects, the dose is effective to produce a Cmax of at least 2 μg/mL, at least 6 μg/mL, or at least 10 μg/mL. In some aspects, the dose is between 0.1-0.6 mg/kg. In some aspects, the dose is 0.1, 0.3, 0.45, or 0.6 mg/kg. In some aspects, the antibody has a half-life of at least 50-100 days

In some aspects, the disclosure provides a method of producing a therapeutic level of an antibody in a human subject in vivo, the method comprising administering (e.g., by intravenous injection or intravenous infusion) to the subject an LNP-formulated mRNA, the mRNA encoding the antibody, in a first dose effective to produce a Cmax of at least 1 μg/mL of the antibody in serum of the subject, and at least one second dose effective to produce a Cmax equal to or greater than a Cmax following the first dose, wherein the LNP comprises Compound 1, a phospholipid, a cholesterol lipid or cholesterol-derivative lipid, and Compound 2. In some aspects, the LNP comprises 20-60 mol % Compound 1 and 0.5-15 mol % Compound 2. In some aspects, the LNP comprises 20-60 mol % Compound 1, 5-25% DSPC, 25-55% cholesterol, and 0.5-15 mol % Compound 2. In some aspects, the LNP comprises 45-49 mol % Compound 1, 8-13 mol % DSPC, 35-40 mol % cholesterol, and 0.5-3.0 mol % Compound 2. In some aspects, the LNP comprises 48-49 mol % Compound 1, 10-12 mol % DSPC, 38-40 mol % cholesterol, and 0.5-2.5 mol % Compound 2. In some aspects, the Cmax following the second dose is at least 1.1-fold, 1.4-fold, or 1.8-fold greater than the Cmax following the first dose. In some aspects, the first and/or at least one second dose is effective to produce a Cmax of at least 2 μg/mL, at least 6 μg/mL, or at least 10 μg/mL. In some aspects, the first and/or at least one second dose is between 0.1-0.6 mg/kg. In some aspects, the first and/or at least one second dose is 0.1, 0.3, 0.45, or 0.6 mg/kg. In some aspects, the antibody has a half-life of at least 50-100 days.

In some aspects, the disclosure provides a method of expressing a therapeutic level of a protein in a human subject in vivo, the method comprising systemically administering to the human subject a dose of LNP-formulated mRNA, the mRNA encoding the therapeutic protein, optionally at a frequency, effective to achieve a therapeutically-effective level of the therapeutic protein in serum or tissue of the subject.

In some aspects, the disclosure provides a method of expressing a therapeutic level of a protein in a human subject in vivo, the method comprising administering to the human subject (e.g., by intravenous injection or intravenous infusion) a dose of LNP-formulated mRNA, the mRNA encoding the therapeutic protein, optionally at a frequency, effective to achieve a therapeutically-effective level of the therapeutic protein in serum or tissue of the subject.

In any of the foregoing aspects, the dose of the LNP-formulated mRNA is effective to maintain a therapeutically-effective level of the therapeutic protein for a duration. In some aspects, the method further comprises administering at least one second dose of the LNP-formulated mRNA to maintain a therapeutically-effective level of the therapeutic protein for a duration.

In some aspects, the disclosure provides methods of administration of an LNP-formulated mRNA encoding a therapeutic protein to human subjects, wherein the therapeutic protein encoded by the mRNA is a secreted protein. In some aspects, the secreted protein is an antibody or antigen binding fragment thereof. In some aspects, the antibody is a prophylactic antibody, optionally for use in protecting the subject against an infectious disease, optionally wherein the subject is naïve for the infectious disease. In some aspects, the antibody comprises an antibody heavy chain (HC) and an antibody light chain (LC), and wherein the HC and LC are encoded on separate mRNAs. In some aspects, the mRNAs encoding the HC and LC are co-formulated in the same LNP, optionally at a 2:1 (HC:LC) w/w ratio. In some aspects, the antibody comprises an engineered single chain antibody, wherein the HC and LC are encoded by a single mRNA.

In some aspects, the disclosure provides methods of administration of an LNP-formulated mRNA encoding a therapeutic protein to human subjects, wherein the therapeutic protein encoded by the mRNA is an intracellular protein. In some aspects, the therapeutic protein encoded by the mRNA is a metabolic enzyme. In some aspects, the metabolic enzyme is a hepatic metabolic enzyme. In some embodiments, the metabolic enzyme is a mitochondrial enzyme. In some aspects, the therapeutic protein encoded by the mRNA is expressed in the liver of the subject. In some aspects, the therapeutic protein is expressed in hepatocytes of the subject.

In some aspects, the disclosure provides methods of administration of an LNP-formulated mRNA encoding a therapeutic protein to human subjects, wherein the human subject is systemically administered a dose of LNP-formulated mRNA once weekly, once every 2 weeks, once every 3 weeks, once every 4 weeks. In some aspects, the dose is between 0.1-0.6 mg/kg. In some aspects, the dose is between 0.2-0.5 mg/kg.

In some aspects, the disclosure provides methods of administration of an LNP-formulated mRNA encoding a therapeutic protein to human subjects, wherein the therapeutically-effective level of the therapeutic protein is in the serum of the human subject. In some aspects, the therapeutically-effective level is a Cmax at least 2-fold, at least 4-fold, at least 6-fold, at least 8-fold, or at least 10-fold higher than a therapeutic threshold level for the therapeutic protein.

In some aspects, the disclosure provides methods of administration of an LNP-formulated mRNA encoding a therapeutic protein to human subjects, wherein the therapeutically-effective level of the therapeutic protein is in a tissue of the human subject. In some aspects, the therapeutically-effective level is an increase in expression or bioactivity of the therapeutic protein or a change in the level of a biomarker (e.g., an increase or decrease in the level of a biomarker) of the therapeutic protein in a tissue

In some aspects, the disclosure provides a method of protecting a human subject against an infectious disease, the method comprising systemically administering to the human subject (e.g., by intravenous injection or intravenous infusion) a dose of LNP-formulated mRNA, the mRNA encoding a prophylactic antibody, optionally at a frequency, effective to achieve and/or maintain a therapeutically-effective level of the prophylactic antibody in serum of the subject. In some aspects, the subject is naïve for the infectious disease.

In some aspects, the LNP-formulated mRNA encodes a prophylactic antibody which comprises an antibody heavy chain (HC) and an antibody light chain (LC) comprising amino acid sequences set forth by SEQ ID NOs: 1 and 3 respectively. In some aspects, the HC and LC are encoded on separate mRNAs. In some aspects, the HC and LC comprise the nucleotide sequences set forth by SEQ ID NOs: 2 and 4 respectively. In some aspects, the mRNAs encoding the HC and LC are co-formulated in the same LNP, optionally at a 2:1 (HC:LC) w/w ratio.

In some aspects, the disclosure provides methods of administration of an LNP-formulated mRNA encoding a therapeutic protein to human subjects, wherein the human subject is systemically administered a dose of LNP-formulated mRNA encoding a prophylactic antibody once weekly, once every 2 weeks, once every 3 weeks, once every 4 weeks. In some aspects, the dose is between 0.1-0.6 mg/kg. In some aspects, the dose is between 0.2-0.5 mg/kg. In some aspects, a therapeutically-effective level is determined by measuring the concentration of the prophylactic antibody in serum collected from the subject. In some aspects, the Cmax of the prophylactic antibody in serum is at least 2 μg/mL, at least 3 μg/mL, at least 4 μg/mL, at least 5 μg/mL, at least 6 μg/mL, at least 7 μg/mL, at least 8 μg/mL, at least 9 μg/mL, or at least 10 μg/mL. In some aspects, the therapeutically-effective level is a Cmax at least 2-fold, at least 4-fold, at least 6-fold, at least 8-fold, or at least 10-fold higher than a therapeutic threshold level for the prophylactic antibody. In some aspects, the therapeutically-effective level is maintained for a duration of at least 12 weeks, at least 13 weeks, at least 14 weeks, at least 15 weeks, at least 16 weeks, at least 18 weeks, at least 19 weeks or at least 20 weeks following systemic administration. In some aspects, the therapeutically-effective levels of the prophylactic antibody in serum are sufficient for neutralization of a target infectious agent.

In some aspects, the disclosure provides a method of expressing a therapeutic level of a hepatic metabolic enzyme in a human subject in vivo, the method comprising systemically administering to the human subject (e.g., by intravenous injection or intravenous infusion) a dose of LNP-formulated mRNA, the mRNA encoding the hepatic metabolic enzyme, optionally at a frequency, effective to achieve a therapeutically-effective level of the hepatic metabolic enzyme in the liver of the subject. In some aspects, the human subject is systemically administered a dose of LNP-formulated mRNA once weekly, once every 2 weeks, once every 3 weeks, once every 4 weeks. In some aspects, the dose of LNP-formulated mRNA is between 0.1-0.6 mg/kg. In some aspects, the therapeutically-effective level of the hepatic metabolic enzyme is in hepatocytes of the human subject. In some aspects, the therapeutically-effective level is measured as a change in the level of expression or bioactivity of the hepatic metabolic enzyme or a change in the level of a substrate of the hepatic metabolic enzyme.

In any of the foregoing aspects or related aspects of the disclosure, the LNP-formulated mRNA is systemically administered to human subjects via intravenous infusion or intravenous injection. In some aspects, the LNP-formulated mRNA is systemically administered via intravenous infusion for a duration of 30 minutes to 4 hours.

In some aspects, the disclosure provides methods of administration of an LNP-formulated mRNA encoding a therapeutic protein to human subjects, wherein the human subject is premedicated or co-medicated with a therapeutic agent.

In any of the foregoing or related aspects of the disclosure, the LNP-formulated mRNA is formulated in an LNP comprising an ionizable amino lipid, a phospholipid, a cholesterol lipid or cholesterol-derivative lipid, a PEG-lipid or conjugated lipid. In some aspects, the ionizable amino lipid is Compound 1. In some aspects, the PEG-lipid is Compound 2. In some aspects, the LNP comprises 50 mol % ionizable amino lipid and 2 mol % PEG lipid. In some aspects, the LNP comprises 50 mol % ionizable amino lipid, 10% DSPC, 38% cholesterol, and 2 mol % PEG lipid.

### BRIEF DESCRIPTION OF THE FIGURES

FIG. 1 is a graph showing the percent survival of AG129 mice intravenously administered 0.5 mg/kg, 0.1 mg/kg, or 0.02 mg/kg of lipid nanoparticle (LNP)-formulated mRNAs encoding the heavy and light chains of the CHIKV-24 antibody, or 0.5 mg/kg of mRNAs expressing a control antibody, over the course of 21 days following challenge with chikungunya virus. The CHIKV-24 antibody clone is a human neutralizing monoclonal IgG antibody against a chikungunya viral protein. The mice were inoculated (challenged) with the virus 24 hours following administration with mRNA.

FIG. 2 is a graph showing the serum concentrations of the mRNA-expressed CHIKV-24 IgG antibody from AG129 mice intravenously administered 0.5 mg/kg, 0.1 mg/kg, or 0.02 mg/kg of LNP-formulated mRNAs encoding the heavy and light chains of CHIKV-24 antibody, or 0.5 mg/kg of mRNAs encoding a control (anti-influenza antibody).

FIG. 3 is a graph showing the serum concentrations of the mRNA-expressed CHIKV-24 IgG antibody over time from cynomolgus monkeys injected intravenously at 0 and 168 hours with 3 mg/kg (top line), 1 mg/kg (middle line), or 0.3 mg/kg (bottom line) of LNP-formulated mRNAs encoding the heavy and light chains of the CHIKV-24 antibody. Control animals received injection of phosphate buffered saline (PBS) only.

FIG. 4 is a schematic detailing a randomized, placebo-controlled, single ascending dose Phase I study in healthy human adults to assess safety, tolerability, and pharmacology of escalating doses of LNP-formulated mRNAs expressing the heavy and light chains of the CHIKV-24 IgG antibody (referred to as LNP-1) administered by intravenous infusion.

FIG. 5 is a graph showing the serum concentrations of the mRNA-expressed CHIKV-24 IgG antibody over time from humans injected with a single intravenous dose of 0.6 mg/kg (top line), 0.3 mg/kg (middle line), or 0.1 mg/kg (bottom line) of LNP-1.

FIG. 6 is a graph showing the percentage of human subjects with neutralizing antibody titers of mRNA-expressed CHIKV-24 antibody following a single intravenous dose of 0.6 mg/kg, 0.3 mg/kg, or 0.1 mg/kg of LNP-1 as compared to a placebo (intravenous administration of 0.9% sodium chloride). Also shown is the geometric mean titer (GMT) measured for serum CHIKV-24 antibody in each cohort, and the number of subjects (N) per cohort.

FIG. 7 is a schematic detailing an amended study design for the Phase I study described in FIG. 4 that was updated to incorporate additional LNP-1 dose level (DL) cohorts. The study design included 7 total cohorts, with each cohort having 3 sentinel subjects administered LNP-1 at the indicated DL with an intervening safety performance evaluation (“IST”), and an expansion cohort having 5 subjects randomly assigned to receive LNP-1 or placebo (3:2 active: placebo). The study design allowed for dose escalation following a review of safety performance (“SMC”). Included in the study design were cohorts at DL1, DL2, and DL3 that were intended to receive no pre-medication with steroids, and an optional cohort at DL4 and cohorts at DL5 and DL6 that were intended to receive pre-medication with steroids.

FIGS. 8A-8B are graphs showing serum concentrations of the CHIKV-24 antibody over time from humans injected with a single intravenous dose of 0.1 mg/kg, 0.3 mg/kg, or 0.6 mg/kg LNP-1, a first and second intravenous dose of 0.3 mg/kg LNP-1 (0.3 mg/kg qWx2), or a single intravenous dose of 0.6 mg/kg LNP-1 following premedication with steroids according to the study design shown in FIG. 7. FIG. 8A provides serum concentrations measured through the duration of the study, and FIG. 8B provides serum concentrations measured in the first 28 days of the study.

### DETAILED DESCRIPTION

As described herein, it has been discovered that systemic administration of mRNA formulated in LNPs can produce therapeutic proteins at therapeutically-effective levels in serum and/or tissues of human subjects. Accordingly, the present disclosure provides compositions (e.g., LNP formulations) comprising mRNA(s) encoding therapeutic polypeptides and methods of use thereof to treat and/or delay progression of a disease or disorder in human subjects.

### Methods of Treatment

The disclosure provides methods for administering an LNP-formulated therapeutic mRNA for use in preventing, treating or delaying progression of a disease or disorder in a clinical, prophylactic or therapeutic application that would benefit from expression of a therapeutic polypeptide encoded by the mRNA. For example, a disease or disorder that would benefit from increased expression of a membrane bound protein, an intracellular protein, or a secreted protein (such as cancer, an autoimmune disease, an infectious disease, a metabolic disease). In one embodiment, the therapeutic polypeptide is an immunogenic protein or polypeptide, e.g., an infectious disease antigen, a tumor cell antigen. In another embodiment, the therapeutic polypeptide is a soluble effector molecule, an antibody, an enzyme, a recruitment factor, a transcription factor, a membrane bound receptor, a membrane bound ligand or any fragment or variant thereof. In some embodiments, the therapeutic polypeptide is a prophylactic antibody for use in protecting a subject against an infectious disease.

### Therapeutic Level

In some embodiments, the disclosure provides methods for systemically administering to a human subject a dose of LNP-formulated mRNA encoding a therapeutic protein, optionally at a frequency to achieve a therapeutically-effective level of the therapeutic protein in serum or tissues of the subject. As used herein, a therapeutic level refers to an amount or concentration of a therapeutic polypeptide translated from an LNP-formulated mRNA within serum or tissue sample of the subject. In a particular embodiment, when the mRNA encodes a secreted protein, the concentration of expressed therapeutic protein is determined in serum. In another embodiment, when the mRNA encodes an intracellular protein, the concentration of expressed therapeutic protein is determined in tissue, e.g., in liver tissue. In another embodiment, when the mRNA encodes an intracellular protein, the concentration of expressed therapeutic protein is determined in a cell population within a tissue, e.g., in liver hepatocytes. In certain embodiments, where the therapeutic protein is an intracellular enzyme, the therapeutic level may be determined indirectly by measuring the effects of the enzyme's activity on the subject's condition.

In some embodiments, a therapeutically-effective level is a therapeutic level that exceeds the therapeutic threshold. The therapeutic threshold refers to the concentration of expressed therapeutic protein that provides the minimum useful therapeutic effect. In some embodiments, the therapeutic threshold is measured in serum or tissue. In some embodiments, the therapeutic threshold as measured in serum is equal to or less than 1 μg/mL, 0.8 μg/mL, 0.6 μg/mL, 0.4 μg/mL or 0.2 μg/mL. In some embodiments, the therapeutic threshold as measured in serum is about 0.2 μg/mL to about 1 μg/mL. In some embodiments, the therapeutic threshold as measured in serum is about 0.2 μg/mL to about 0.5 μg/mL.

In some embodiments, the therapeutically-effective level is a Cmax at least 2-fold, at least 4-fold, at least 6-fold, at least 8-fold, or at least 10-fold higher than a therapeutic threshold level for the therapeutic protein. As used herein, Cmax refers to the maximum (or peak) concentration of a therapeutic polypeptide in serum or a tissue sample obtained from a subject after administration of a first dose of the LNP-formulated mRNA and prior to administration of a subsequent dose. As is understood by one of skill in the art, the Cmax may be measured following each dose administered to the subject (e.g., the first, second, third, fourth, etc dose). The Tmax refers to the time at which the maximum concentration (Cmax) is observed relative to the time of administration. In some embodiments, the Tmax is about 30 hours, about 35 hours, about 40 hours, about 45 hours, or about 50 hours. In some embodiments, the Tmax is about 35-50 hours.

In some embodiments, the therapeutically-effective level has a Cmax (e.g., as measured in serum) that is at least 1 μg/mL, 2 μg/mL, 3 μg/mL, 4 μg/mL or 5 μg/mL. In some embodiments, the Cmax (e.g., as measured in serum) is equal to or less than 6 μg/mL, 7 μg/mL, 8 μg/mL, 9 μg/mL or 10 μg/mL. In some embodiments, the Cmax (e.g., as measured in serum) is equal to or less than 10 μg/mL, 012 μg/mL, 014 μg/mL, 16 μg/mL, 18 μg/mL or 20 μg/mL.

In some embodiments, the duration of the therapeutically-effective level is dependent on the half-life of the expressed therapeutic polypeptide (e.g., antibody) after administration of a dose of LNP-formulated mRNA. As used herein, “half-life” refers to the time interval over which the concentration of the therapeutic polypeptide in the body is decreased by one-half from the maximal concentration, for example, as measured in serum or a tissue sample obtained from a subject following administration of a first dose of the LNP-formulated mRNA and prior to administration of a subsequent dose. In some embodiments, the therapeutic polypeptide (e.g., antibody) has a half-life of at least 50 days, 55 days, 60 days, 65 days, 70 days, 75 days, or 80 days following administration. In some embodiments, the therapeutic polypeptide (e.g., antibody) has a half-life of approximately 50-100 days following administration.

Methods of determining a therapeutic threshold are known in the art and depend upon the therapeutic polypeptide. In some embodiments, the therapeutic threshold is determined for a therapeutic polypeptide that is an infectious disease antibody, wherein data is collected from subjects having existing immunity to an infectious disease antigen targeted by the antibody, and wherein the therapeutic threshold is determined to be substantially equivalent to the concentration of the infectious disease antibody in the serum of subjects immune to re-infection with the infectious disease. As another example, the therapeutic threshold for a therapeutic intracellular protein, for example, in an enzyme deficiency disorder or disease, can be determined based on data collected from normal human subjects. A method of preventing and/or treating a disease or disorder that would benefit from increased expression of a therapeutic polypeptide comprises systemically administering to the human subject a dose of an LNP-formulated mRNA that comprises an ORF encoding the therapeutic polypeptide, optionally at a frequency and/or duration, effective to achieve and/or maintain a therapeutically-effective level of the therapeutic polypeptide in the serum and/or tissue of the human subject.

### Human Subject

In some embodiments, the LNP-formulated mRNA of the disclosure is used to treat and/or prevent diseases, disorders or conditions in human subjects that would benefit by achieving a therapeutically-effective level of the therapeutic protein (e.g., by increased expression of a therapeutic polypeptide) and/or by maintaining a therapeutically-effective level of a therapeutic polypeptide in the serum and/or tissue of the human subject. In some embodiments, the LNP-formulated mRNAs of the disclosure are systemically administered to treat and/or prevent a disease or disorder resulting from a deficiency of a polypeptide of interest (e.g., a membrane bound, intracellular, or secreted protein). In some embodiments, the LNP-formulated mRNAs of the disclosure are used to prevent a disease or disorder (e.g., an infectious disease or disorder) in a human subject by systemically administering a dose of an LNP-formulated mRNA encoding a therapeutic polypeptide (e.g., a prophylactic antibody that selectively binds an infectious agent) to achieve a therapeutically-effective level of the therapeutic polypeptide to protect the subject against the disease or disorder (e.g., a disease or disorder caused by an infectious agent).

In some embodiments, systemic administration of an LNP-formulated mRNA of the disclosure achieves and/or maintains a therapeutically-effective level of a biomarker of the therapeutic protein encoded by the mRNA, e.g., changes the level of a biomarker of a therapeutic polypeptide (e.g., changes the level of one or more substrates of the therapeutic polypeptide) in a subject in need thereof. In some embodiments, the LNP-formulated mRNA of the present disclosure are systemically administered to human subjects to reduce the level of a metabolite associated with a disease or disorder, the method comprising administering to the subject a dose of an LNP-formulated mRNA encoding the therapeutic polypeptide (e.g., a membrane bound, intracellular, or secreted protein), optionally at frequency, effective to achieve and/or maintain a therapeutically-effective level of the therapeutic polypeptide in the serum and/or tissue of the human subject.

In one embodiment, the disclosure provides a method for treating a disease or disorder that would benefit from increased expression of a therapeutic polypeptide (e.g., a membrane bound, intracellular, or secreted protein), comprising: (a) systemically administering to a patient in need thereof one or more doses of LNP-formulated mRNA encoding a therapeutic polypeptide (e.g., a membrane bound, intracellular, or secreted protein) or a pharmaceutical composition thereof; and (b) monitoring the concentration of the therapeutic polypeptide (e.g., detected in biological specimens such as plasma, serum, cerebral spinal fluid, urine, or other biofluids). In some embodiments, an increase in the concentration of the therapeutic polypeptide following administration of the LNP-formulated mRNA encoding the therapeutic polypeptide (e.g., a membrane bound, intracellular, or secreted protein) or pharmaceutical composition thereof indicates that the course of treatment is effective for treating the disease or disorder that would benefit from increased expression of a therapeutic polypeptide (e.g., a membrane bound, intracellular, or secreted protein).

In some embodiments, the methods for treating a disease or disorder that would benefit from systemic administration of an LNP-formulated mRNA to achieve and/or maintain a therapeutically-effective level of a therapeutic polypeptide e.g., an increased expression of the therapeutic polypeptide (e.g., a membrane bound, intracellular, or secreted protein) provided herein alleviate or manage one, two or more symptoms associated with the disease or disorder. In some embodiments, alleviating or managing one, two or more symptoms of a disease or disorder is used as a clinical endpoint for efficacy of an LNP-formulated mRNA. In some embodiments, the methods for treating a disease or disorder provided herein reduce the duration and/or severity of one or more symptoms associated with the disease or disorder. In some embodiments, the methods for treating a disease or disorder provided herein inhibit the onset, progression and/or recurrence of one or more symptoms associated with the disease or disorder. In some embodiments, the methods for treating a disease or disorder provided herein reduce the number of symptoms associated with the disease or disorder. In some embodiments, the methods for treating a disease or disorder provided herein inhibit or reduce the progression of one or more symptoms associated therewith.

In some embodiments, the methods provided herein increase the survival of a patient diagnosed with a disease or disorder. In some embodiments, the methods for treating a disease or disorder provided herein reduce the mortality of subjects diagnosed with the disease or disorder. In some embodiments, the methods for treating a disease or disorder provided herein increase symptom-free survival of patients having the disease or disorder. In some embodiments, the methods for treating a disease or disorder provided herein do not cure the disease or disorder in patients but prevent the progression or worsening of the disease. In some embodiments, the methods for treating a disease or disorder provided herein enhance or improve the therapeutic effect of another therapy.

In some embodiments, the disclosure provides methods for treating a disease or disorder that would benefit from systemic administration of an LNP-formulated mRNA to achieve and/or maintain a therapeutically-effective level of an antibody (e.g., prophylactic antibody), to alleviate or manage one or more symptoms associated with the disease or disorder.

In some embodiments, the disclosure provides methods for preventing the onset or development of a nosocomial infection resulting from an infectious agent in a human subject, wherein the method comprises systemically administering to the human subject a dose of LNP-formulated mRNA, wherein the LNP-formulated mRNA comprises one or more mRNAs encoding a prophylactic antibody targeting the infectious agent, wherein the dose is administered prior the subject being admitted to a hospital for an inpatient procedure (e.g., a surgical procedure), and wherein the method is effective to achieve a therapeutically-effective level of the prophylactic antibody in serum of the subject. In some embodiments, the subject is uninfected with or naïve to the nosocomial infection prior to being admitted. In some embodiments, the dose of LNP-formulated mRNA is administered prior to the subject being admitted to the hospital, e.g., about 48 hours, 72 hours, 1 week, 2 weeks, or 3 weeks prior to being admitted to the hospital. In some embodiments, the method achieves a therapeutically-effective level of the antibody in serum that is maintained for a duration that extends beyond the length of time the subject is admitted to the hospital and/or the length of time for the subject to recover from the procedure. In some embodiments, the therapeutically-effective level is sufficient for neutralization of the infectious agent upon exposure.

In some embodiments, the disclosure provides methods for preventing the onset or development of a seasonal respiratory virus in a human subject, the method comprising systemically administering to the human subject a dose of LNP-formulated mRNA, wherein the LNP-formulated mRNA comprises one or more mRNAs encoding a prophylactic antibody targeting the respiratory virus, wherein the dose is administered prior to or concurrent with a seasonal epidemic caused by the virus, and wherein the method is effective to achieve a therapeutically-effective level of the prophylactic antibody in serum of the subject for a duration. In some embodiments, the duration extends beyond a length of time for the seasonal epidemic. In some embodiments, the subject has one or more criteria that result in increased susceptibility to the seasonal respiratory virus. In some embodiments, the criteria is an age greater than 65 years old or less than 5 years old. In some embodiments, the criteria is pregnancy, or a pre-existing condition selected from heart disease, cancer, or an autoimmune disorder. In some embodiments, the prophylactic antibody in serum is sufficient for neutralization of the respiratory virus.

In some embodiments, the disclosure provides a method of protecting a human subject against an endemic infectious disease, the method comprising systemically administering to the human subject a dose of LNP-formulated mRNA, wherein the LNP-formulated mRNA comprises one or more mRNAs encoding a prophylactic antibody targeting the infectious disease, wherein the dose is administered prior to the subject traveling to a region with a high prevalence of the disease, and wherein the method is effective to achieve a therapeutically-effective level of the prophylactic antibody in serum of the subject for a duration. In some embodiments, the duration extends beyond a length of time for traveling to the region. In some embodiments, the dose is administered at least 36, 48, or 72 hours prior to traveling. In some embodiments, the dose is administered no more than 7, 10, 13, 16, 18, or 21 days prior to traveling. In some embodiments, the prophylactic antibody in serum is sufficient for neutralization of the infectious agent.

In some embodiments, the disclosure provides a method of expressing a therapeutic level of a therapeutic antibody against an inflammatory cytokine in a human subject in vivo, the method comprising systemically administering to the subject a dose of LNP-formulated mRNA, the mRNA encoding the therapeutic antibody, effective to achieve a therapeutically-effective level of the therapeutic antibody in serum of the subject. In some embodiments, the subject is at risk of a cytokine storm. In some embodiments, the risk is a result of an infection, an autoimmune disorder, or administration of an immunotherapy. In some embodiments, the dose is administered prior to the risk of the cytokine storm.

In some embodiments, the subject is a male human. In some embodiments, the subject is a female human. In some embodiments, a subject treated for a disease or disorder in accordance with the methods provided herein is a human infant, an elderly human, an adult human and/or a human child. In some embodiments, a subject treated for a disease or disorder in accordance with the methods provided herein is a human that is at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, or 55 years of age.

In some embodiments, a subject treated for a disease or disorder in accordance with the methods provided herein is administered an LNP-formulated mRNA before any adverse effects or intolerance to therapies other than the LNP-formulated mRNA develops. In some embodiments, a subject treated for a disease or disorder in accordance with the methods provided herein is a refractory patient. In a certain embodiment, a refractory patient is a patient that is refractory to a standard therapy. In some embodiments, a subject treated for a disease or disorder in accordance with the methods provided herein is a human that has proven refractory to therapies other than treatment with an LNP-formulated mRNA but is no longer on these therapies. In some embodiments, a subject treated for a disease or disorder in accordance with the methods provided herein is a human already receiving one or more conventional therapies.

In some embodiments, a subject treated for a disease or disorder in accordance with the methods provided herein is a human susceptible to adverse reactions to conventional therapies. In some embodiments, a subject treated for a disease or disorder in accordance with the methods provided herein is a human that has not received a therapy, e.g., a prior to the administration of an LNP-formulated mRNA. In some embodiments, a subject treated for a disease or disorder in accordance with the methods provided herein is a human that has received a therapy prior to administration of an LNP-formulated mRNA. In some embodiments, a subject treated for a disease or disorder in accordance with the methods provided herein is a human that has experienced adverse side effects to the prior therapy or the prior therapy was discontinued due to unacceptable levels of toxicity to the human.

### Administration and Dosage

In some embodiments, the disclosure pertains to a method of increasing expression of a therapeutic polypeptide in a subject in need thereof, the method comprising administering to the subject a composition of the disclosure comprising an LNP-formulated mRNA comprising an ORF encoding the therapeutic polypeptide.

In some embodiments, a dose of LNP-formulated mRNA is administered by parenteral administration, optionally at a frequency and/or duration to achieve and/or maintain a therapeutically-effective level of the therapeutic polypeptide in the serum and/or tissue of the human subject. In some embodiments, a dose of LNP-formulated mRNA is administered by intravenous administration, optionally at a frequency and/or duration, effective to achieve and/or maintain a therapeutically-effective level of the therapeutic polypeptide in the serum and/or tissue of the human subject. In some embodiments, a dose of LNP-formulated mRNA is administered by continuous infusion, for example, using a catheter, optionally at a frequency and/or duration, effective to achieve and/or maintain a therapeutically-effective level of the therapeutic polypeptide in the serum and/or tissue of the human subject.

In some embodiments, the subject is provided with or administered a pharmaceutical composition of the disclosure comprising the LNP-formulated mRNA. In some embodiments, the LNP-formulated mRNA, or pharmaceutical composition is administered to the patient parenterally (e.g., intravenous administration). In particular embodiments, the subject is a mammal, e.g., a human. In various embodiments, the subject is provided with a therapeutically effective amount of the LNP-formulated mRNA.

A pharmaceutical composition including one or more therapeutic mRNAs of the disclosure may be administered to a subject by any suitable route. In some embodiments, compositions of the disclosure are administered by one or more of a variety of routes, including parenteral (e.g., subcutaneous, intracutaneous, intravenous, intraperitoneal, intramuscular, intraarticular, intraarterial, intrasynovial, intrasternal, intrathecal, intralesional, or intracranial injection, as well as any suitable infusion technique), oral, trans- or intra-dermal, interdermal, rectal, intravaginal, topical (e.g., by powders, ointments, creams, gels, lotions, and/or drops), mucosal, nasal, buccal, enteral, vitreal, intratumoral, sublingual, intranasal; by intratracheal instillation, bronchial instillation, and/or inhalation; as an oral spray and/or powder, nasal spray, and/or aerosol, and/or through a portal vein catheter. In some embodiments, a composition may be administered intravenously, intramuscularly, intradermally, intra-arterially, intratumorally, subcutaneously, or by inhalation. However, the present disclosure encompasses the delivery of compositions of the disclosure by any appropriate route taking into consideration likely advances in the sciences of drug delivery. In general, the most appropriate route of administration will depend upon a variety of factors including the nature of the pharmaceutical composition including one or more mRNAs (e.g., its stability in various bodily environments such as the bloodstream and gastrointestinal tract), and the condition of the patient (e.g., whether the patient is able to tolerate particular routes of administration).

In some embodiments, the LNP-formulated mRNA of the disclosure is systemically administered to human subjects at a dose or dosage level effective to achieve and/or maintain a therapeutically-effective level of a therapeutic protein in the serum and/or tissue of the human subject. In some embodiments, the LNP-formulated mRNA is systemically administered to human subjects at a dose of about 0.1 to 1 mg/kg of mRNA or a pharmaceutical composition thereof (e.g., a solution formulated for intravenous infusion). In some embodiments, the LNP-formulated mRNA is systemically administered to human subjects at a dose of about 0.1 mg/kg of mRNA or a composition thereof. In some embodiments, the LNP-formulated mRNA is systemically administered to human subjects at a dose of about 0.2 mg/kg of mRNA or a composition thereof. In some embodiments, the LNP-formulated mRNA is systemically administered to human subjects at a dose of about 0.3 mg/kg of mRNA or a composition thereof. In some embodiments, the LNP-formulated mRNA is systemically administered to human subjects at a dose of about 0.4 mg/kg of mRNA or a composition thereof. In some embodiments, the LNP-formulated mRNA is systemically administered to human subjects at a dose of about 0.5 mg/kg of mRNA or a composition thereof. In some embodiments, the LNP-formulated mRNA is systemically administered to human subjects at a dose of about 0.6 mg/kg of mRNA or a composition thereof. In some embodiments, the LNP-formulated mRNA is systemically administered to human subjects at a dose of about 0.7 mg/kg of mRNA or a composition thereof. In some embodiments, the LNP-formulated mRNA is systemically administered to human subjects at a dose of about 0.8 mg/kg of mRNA or a composition thereof. In some embodiments, the LNP-formulated mRNA is systemically administered to human subjects at a dose of about 0.9 mg/kg of mRNA or a composition thereof. In some embodiments, the LNP-formulated mRNA is systemically administered to human subjects at a dose of about 1 mg/kg of mRNA or a composition thereof.

In some embodiments, the LNP-formulated mRNA is systemically administered to human subjects at a dose, frequency and/or duration to achieve and/or maintain a therapeutically effective level of a therapeutic polypeptide in the serum and/or tissue of the subject. In some embodiments, a dose of LNP-formulated mRNA is administered one time to a given subject. In some embodiments, a dose of LNP-formulated mRNA is administered at a frequency effective to achieve a therapeutically-effective level of a therapeutic polypeptide. For example, the human subject is as administered multiple doses (e.g., 2, 3, 4, or 5) of the LNP-formulated mRNA effective to achieve a therapeutically-effective level of the therapeutic polypeptide in the serum and/or tissue of the subject. In some embodiments, a dose of LNP-formulated mRNA is administered for a duration effective to achieve a therapeutically-effective level of a therapeutic polypeptide. For example, the human subject is administered multiple doses (e.g., 2, 3, 4, or 5) of the LNP-formulated mRNA for a duration (e.g., one week, 2 weeks, 3 weeks, 1 month, 2 months, 3 months, 4-6 months, 6-12 months, 1-5 years or longer) effective to achieve a therapeutically-effective level of the therapeutic polypeptide in the serum and/or tissue of the subject.

In some embodiments, a dose of LNP-formulated mRNA is systemically administered to human subjects as multiple doses (e.g., 2, 3, 4, or 5) for an extended duration. For example, in some embodiments, the human subject is administered a dose of the LNP-formulated mRNA at a frequency of once weekly, bi-weekly, once monthly, or bi-monthly for a duration (e.g., one week, 2 weeks, 3 weeks, 1 month, 2 months, 3 months, 4-6 months, 6-12 months, 1-5 years or longer) effective to achieve a therapeutically-effective level of the therapeutic polypeptide in the serum and/or tissue of the subject. In some embodiments, the human subject is administered a dose of the LNP-formulated mRNA at a frequency of 1, 2, 3, 4, or 5 times per week for a duration (e.g., one week, 2 weeks, 3 weeks, 1 month, 2 months, 3 months, 4-6 months, 6-12 months, 1-5 years or longer) effective to achieve a therapeutically-effective level of the therapeutic polypeptide in the serum and/or tissue of the subject. In some embodiments, the human subject is administered a dose of the LNP-formulated mRNA at a frequency of 1, 2, 3, 4, or 5 times per month for a duration (e.g., one week, 2 weeks, 3 weeks, 1 month, 2 months, 3 months, 4-6 months, 6-12 months, 1-5 years or longer) effective to achieve a therapeutically-effective level of the therapeutic polypeptide in the serum and/or tissue of the subject.

A dose may be administered one or more times per day, in the same or a different amount, to obtain a desired level of mRNA expression and/or effect (e.g., a therapeutic effect). The desired dosage may be delivered, for example, three times a day, two times a day, once a day, every other day, every third day, every week, every two weeks, every three weeks, or every four weeks. In some embodiments, the desired dosage may be delivered using multiple administrations (e.g., two, three, four, five, six, seven, eight, nine, ten, eleven, twelve, thirteen, fourteen, or more administrations) to achieve a therapeutically-effective level of the therapeutic polypeptide in the serum and/or tissue of the subject. For example, in some embodiments, a composition of the disclosure is administered at least two times wherein the second dose is administered at least one day, or at least 3 days, or least 7 days, or at least 10 days, or at least 14 days, or at least 21 days, or at least 28 days, or at least 35 days, or at least 42 days or at least 48 days after the first dose is administered. In certain embodiments, a first and second dose are administered on days 0 and 2, respectively, or on days 0 and 7 respectively, or on days 0 and 14, respectively, or on days 0 and 21, respectively, or on days 0 and 48, respectively. Additional doses (i.e., third doses, fourth doses, etc.) can be administered on the same or a different schedule on which the first two doses were administered. For example, in some embodiments, the first and second dosages are administered 7 days apart and then one or more additional doses are administered weekly thereafter. In another embodiment, the first and second dosages are administered 7 days apart and then one or more additional doses are administered every two weeks thereafter.

In some embodiments, the LNP-formulated mRNA is systemically administered to human subjects at a dose, frequency and/or duration sufficient to achieve and/or maintain a therapeutically effective level of a therapeutic polypeptide in the serum and/or tissue of the subject for at least 4-8, 4-10, 4-12, 8-12, 8-16, 8-20, 10-20, or 10-30 weeks. In some embodiments, the LNP-formulated mRNA is systemically administered to human subjects at a dose, frequency and/or duration sufficient to achieve and/or maintain a therapeutically effective level of a therapeutic polypeptide in the serum and/or tissue of the subject for at least 4 weeks, 5 weeks, 6 weeks, 7 weeks, 8 week, 9 week, 10 week, 11 weeks, 12 weeks, 13 week, 14 week, 15 weeks, 16 weeks, 17 weeks, or 18 weeks.

In some embodiments, a human subject is administered a first dose and at least one second or subsequent dose (e.g., a repeat dose) of an LNP-formulated mRNA of the disclosure to produce a therapeutically-effective level of an encoded therapeutic polypeptide (e.g., antibody) in serum of the subject. In some embodiments, administration of the at least one second dose or subsequent dose are administered to the human subject separately and subsequent to the first dose.

In some embodiments, a human subject is administered a split dose of LNP-formulated mRNA of the disclosure, wherein the split dose comprises a first dose and at least one second dose, wherein the first dose and the at least one second dose are in equal amount (the same dose) or different amounts, and wherein the total amount of LNP-formulated mRNA provided by the first dose and the second dose provide a therapeutically-effective amount of LNP-formulated mRNA to the human subject. In some embodiments, the split dose of LNP-formulated mRNA administered to the subject provides a therapeutically-effective amount of not more than 0.6 mg/kg. In some embodiments, the split dose of LNP-formulated mRNA is administered as a first dose on day 0 and a second dose administered about 1 week, about 2 weeks, about 3 weeks, or about 4 weeks following the first dose, optionally wherein the split dose of LNP-formulated mRNA provides a therapeutically-effective amount of not more than 0.6 mg/kg. In one embodiment, the human subject is administered a split dose of LNP-formulated mRNA of the disclosure wherein the split dose comprises a first dose of 0.3 mg/kg and a second dose of 0.3 mg/kg, optionally wherein the first dose is administered at day 0 and the second dose is administered at about 1 week following the first dose.

In some embodiments, a human subject is administered a first loading dose and at least one second or subsequent dose (e.g., a repeat or maintenance dose) of an LNP-formulated mRNA of the disclosure to produce a therapeutically-effective level of an encoded therapeutic polypeptide (e.g., antibody) in serum of the subject. In some embodiments, administration of the at least one second dose or subsequent dose are administered to the human subject separately and subsequent to the first dose.

In some embodiments, administration of the first dose is followed by a second dose. In some embodiments, the first dose and the second dose are each administered by the same route of administration (e.g., by intravenous injection or intravenous infusion) or by a different route of administration. In some embodiments, the first dose and the second dose contain the same or a different amount of LNP-formulated mRNA. In some embodiments, the second dose is administered at 1 week, 2 weeks, 3 weeks, or 4 weeks following administration of the first dose.

In some embodiments, administration of the first dose is followed by more than one second or subsequent doses, wherein the human subject is repeatedly administered two, three, four or five second or subsequent doses of LNP-formulated mRNA at a frequency and for a duration of time relative to administration of the first dose. In some embodiments, the subsequent doses are maintenance doses to maintain the therapeutic level of the therapeutic protein. In some embodiments, the first dose and second or subsequent doses are each administered by the same route of administration (e.g., by intravenous injection or intravenous infusion) or by a different route of administration. In some embodiments, the first dose and the second or subsequent doses contain the same or a different amount of LNP-formulated mRNA. In some embodiments, the second or subsequent or maintenance doses are administered at a frequency of once per week, once per every two weeks, once per every three weeks, or once per every four weeks relative to administration of the first dose.

In some embodiments, the first dose and at least one second or subsequent dose is administered at a frequency and/or for a duration to maintain a therapeutic level of the therapeutic polypeptide in serum (e.g., above a therapeutic threshold level for the therapeutic polypeptide, e.g., 1 μg/mL or higher) for at least 12 weeks, at least 13 weeks, at least 14 weeks, at least 15 weeks, at least 16 weeks, at least 18 weeks, at least 19 weeks, at least 20 weeks, at least 21 weeks, at least 22 weeks, at least 23 weeks, or at least 24 weeks. In some embodiments, the first dose and at least one second or subsequent dose comprises a first dose of LNP-formulated mRNA is between 0.1-0.6 mg/kg. In some embodiments, the first dose is 0.1, 0.3, 0.45, or 0.6 mg/kg. In some embodiments, the first dose is not more than 0.6 mg/kg. In some embodiments, the at least one second or subsequent dose of LNP-formulated mRNA is between 0.1-0.6 mg/kg. In some embodiments, the at least one second or subsequent dose is 0.1, 0.3, 0.45, or 0.6 mg/kg. In some embodiments, the at least one second or subsequent dose is not more than 0.6 mg/kg.

In some embodiments, the first dose and at least one second or subsequent dose provides a therapeutic level of the therapeutic protein in serum having a Cmax following the first dose that is (i) at least 2-10 μg/mL; and/or (ii) is at least 2-fold to at least 10-fold higher than a therapeutic threshold level for the therapeutic protein. In some embodiments, the therapeutic level has a Cmax following the at least one second or subsequent dose that is equal to or greater than the Cmax following the first dose. In some embodiments, the Cmax following the at least one second or subsequent dose is at least 1.1-fold, 1.4-fold, or 1.8-fold greater than the Cmax following the first dose.

Suitable doses of an LNP-formulated mRNA for human patients can be evaluated in, e.g., a Phase I dose escalation study. Data obtained from the cell culture assays and animal studies can be used in formulating a range of dosage for use in humans. The dosage of such LNP-formulated mRNA described herein lies generally within a range of local concentrations of the LNP-formulated mRNA that include the ED50 with little or no toxicity. The dosage may vary within this range depending upon the dosage form employed and the route of administration utilized. For the mRNA and compositions described herein, the therapeutically effective dose can be estimated initially from cell culture assays. A dose can be formulated in animal models to achieve a therapeutically effective concentration within the local site that includes the IC50 (i.e., the concentration of the mRNA which achieves a half-maximal inhibition of symptoms) as determined in cell culture. Such information can be used to more accurately determine useful doses in humans.

Also provided herein are methods for reducing drug responses, including dose-limiting toxicity, associated with LNP-formulated mRNAs. Further description of methods to reduce dose-limiting toxicity associated with LNPs is provided by U.S. Pat. No. 10,207,010, which is incorporated by reference herein. In some embodiments, a dose lower than a certain therapeutic ceiling is used to reduce dose-limiting toxicities or to prevent their occurrence. As used herein, the “therapeutic celling” refers to the concentration of expressed therapeutic protein at which the maximum tolerated side effects occur.

In some embodiments, a method for reducing dose-limiting toxicities associated with LNP-formulated mRNA is performed by at administering to a subject in need thereof a dose of the LNP-formulated mRNA wherein the dose is equal to or less than about 0.3 mg/kg of mRNA. In some embodiments, a method for reducing dose-limiting toxicities associated with LNP-formulated mRNA is performed by at administering to a subject in need thereof a dose of the LNP-formulated mRNA wherein the dose is equal to or less than about 0.6 mg/kg of mRNA. In some embodiments, the dose can range from about 0.1 to about 0.3 mg/kg of mRNA. In some embodiments, the dose can range from about 0.1 to about 0.6 mg/kg of mRNA. In some embodiments wherein a first and second dose are administered, the interval between the first and second dose is at least 2 weeks, at least 10 days, at least 1 week, at least 4 days, or at least 2 days. In some embodiments, treatment regimens are used to maintain serum levels of LNPs comprising mRNAs of the disclosure below the therapeutic ceiling for triggering clinically significant dose-limiting toxicities to reduce such toxicity or prevent their occurrence.

In some embodiments, the dose-limiting toxicities associated with LNP-formulated mRNA are reduced or substantially eliminated by administering a therapeutically-effective amount of LNP-formulated mRNA to a human subject in two or more doses, wherein the therapeutically-effective amount of LNP-formulated mRNA is administered not in a single dose, but in two or more doses to the human subject over a duration of time (e.g., hours, days weeks). In some embodiments, the therapeutically-effective amount of LNP-formulated mRNA is not more than 1 mg/kg, wherein the therapeutically-effective amount is administered in a first dose and at least one second or subsequent dose, wherein the first and at least one second or subsequent doses are each independently equal to or less than about 0.6 mg/kg of mRNA (e.g., between about 0.1 mg/kg and about 0.6 mg/kg of mRNA). In some embodiments, a therapeutically-effective amount of LNP-formulated mRNA administered as two or more doses provides reduced or substantially eliminated dose-limiting toxicities and/or a more tolerable safety profile as compared to the therapeutically-effective amount of LNP-formulated mRNA administered as a single dose.

In some embodiments, the dose-limiting toxicities associated with LNP-formulated mRNA are reduced or substantially eliminated by administering a therapeutically-effective amount of LNP-formulated mRNA to a human subject in a split dose, wherein the therapeutically-effective amount of LNP-formulated mRNA is administered in a first dose and at least one second dose, wherein a first dose (e.g., one-half) of the therapeutically-effective amount is administered to the subject and at least one second dose (e.g., one-half) of the therapeutically-effective amount is administered a period of time following the first dose (e.g., hours, days, weeks). In some embodiments, the at least one second dose is administered about 1 week, about 2 weeks, about 3 weeks, or about 3 weeks following the first dose. In some embodiments, the therapeutically-effective amount of LNP-formulated mRNA is not more than 0.6 mg/kg. In some embodiments, a therapeutically-effective amount of LNP-formulated mRNA administered as a split dose provides reduced or substantially eliminated dose-limiting toxicities and/or a more tolerable safety profile as compared to the therapeutically-effective amount of LNP-formulated mRNA administered as a single dose.

In some aspects, the at least one second or subsequent dose is administered about 1 week, about 2 weeks, about 3 weeks, or about 4 weeks or more following administration of the first dose of LNP-formulated mRNA. In some embodiments, the second dose is administered at a frequency of once per week, once per every 2 weeks, once per every 3 weeks, or once per every 4 weeks.

In some embodiments, the at least one second or subsequent dose of LNP-formulated mRNA is sufficient to achieve a tolerable safety profile following systemic administration to the human subject.

In some embodiments, the at least one second or subsequent dose of LNP-formulated mRNA is delivered to achieve a particular therapeutic level of a therapeutic protein following systemic administration of an initial dose to the human subject.

In some embodiments, the LNP-formulated mRNA is administered as a single dose or as two or more doses (which may or may not contain the same amount of the desired molecule) over time, or as a continuous infusion via an implantation device or catheter. Further refinement of the appropriate dosage is routinely made by those of ordinary skill in the art. In certain embodiments, appropriate dosages can be ascertained through use of appropriate dose-response data. In some embodiments, the specified time period is determined by a clinician.

In some embodiments, the dose comprising LNP-formulated mRNA is administered as a continuous infusion. It is within the knowledge of those skilled in the art to select the duration of administration of the LNP-formulated mRNA (e.g., infusion) so as to maintain the serum level of the LNPs below a therapeutic ceiling. For example, when a large dose is needed to reach the intended therapeutic effects, a longer administration period is used. Occurrence of any dose-limiting toxicities can be monitored via conventional approaches in medical practice. The dose and administration are adjusted upon the signs of symptoms associated with toxicity. In some embodiments, the continuous infusion is administered over 0.5-1 hours, 0.5-2 hours, 0.5-3 hours, 1-3 hours, or 1-4 hours. In some embodiments, the continuous infusion is administered over about 0.5, 1, 1.5, 2, 2.5, 3, 3.5 or 4 hours. In some embodiments, the continuous infusion is administered over about 0.5 hours. In some embodiments, the continuous infusion is administered over about 1 hour. In some embodiments, the continuous infusion is administered over about 2 hours. In some embodiments, the continuous infusion is administered over about 3 hours. In some embodiments, the continuous infusion is administered over about 4 hours.

In some embodiments, the dosing regimen is determined by the pharmacodynamics effects of the therapeutic polypeptide. In some embodiments, the frequency of dosing will take into account the pharmacokinetic parameters of the mRNA in the LNP formulation used. In certain embodiments, a clinician will administer the composition until a dosage is reached that achieves or maintains the desired effect. In some embodiments, achievement of a desired effect occurs immediately after administration of a dose. In some embodiments, achievement occurs at any point in time following administration. In some embodiments, achievement occurs at any point in time during a dosing interval. In some embodiments, achievement of a desired effect is determined by analyzing a biological sample (e.g., serum sample, e.g., biopsy sample) immediately after administration of a dose, at any point in time following administration of a dose, at any point in time during a doing interval, or combinations thereof.

### Combination Therapy

In some embodiments, an LNP-formulated mRNA of the disclosure may be administered in combination with another agent, for example, another therapeutic agent, a prophylactic agent, and/or a diagnostic agent. By “in combination with,” it is not intended to imply that the agents must be administered at the same time and/or formulated for delivery together, although these methods of delivery are within the scope of the present disclosure. For example, one or more compositions, including one or more different LNP-formulated mRNAs, may be administered in combination. Compositions can be administered concurrently with, prior to, or subsequent to, one or more other desired therapeutics or medical procedures. In general, each agent will be administered at a dose and/or on a time schedule determined for that agent. In some embodiments, the present disclosure encompasses the delivery of compositions of the disclosure in combination with agents that improve their bioavailability, reduce and/or modify their metabolism, inhibit their excretion, and/or modify their distribution within the body.

The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, a composition useful for treating cancer may be administered concurrently with a chemotherapeutic agent), or they may achieve different effects (e.g., control of any adverse effects).

Presented herein are combination therapies for the treatment of a disease or disorder that involve the administration of an LNP-formulated mRNA encoding a therapeutic polypeptide in combination with one or more additional therapies (e.g., secondary agents) to a subject in need thereof. In a specific embodiment, presented herein are combination therapies for the treatment of a disease or disorder which involve the administration of an effective amount of an LNP-formulated mRNA encoding a therapeutic polypeptide in combination with an effective amount of another therapy (e.g., secondary agent) to a subject in need thereof. In some embodiments, the combination therapies provided herein involve administering to a subject in need thereof an LNP-formulated mRNA in combination with conventional, or known, therapies for a given disease or disorder. In some embodiments, other therapies for a disease or disorder (e.g., secondary agents) are aimed at controlling or relieving symptoms associated with the disease or disorder and/or administration of the LNP-formulated mRNA.

In some embodiments, the methods for treating a disease or disorder provided herein comprise administering an LNP-formulated mRNA as a single agent for a period of time prior to administering the LNP-formulated mRNA in combination with an additional therapy. In some embodiments, the methods for treating a disease or disorder provided herein comprise administering an additional therapy alone for a period of time prior to administering an LNP-formulated mRNA in combination with the additional therapy. In some embodiments, the methods for treating a disease or disorder provided herein comprise administering an additional therapy alone for a period of time prior to administering an LNP-formulated mRNA alone. In some embodiments, the methods for treating a disease or disorder provided herein comprise administering an LNP-formulated mRNA alone for a period of time prior to administering an additional therapy alone.

In some embodiments, administration of an LNP-formulated mRNA is performed substantially simultaneously with administration of another therapy (e.g., a secondary agent). Substantially simultaneously is meant administration of the LNP-formulated mRNA is performed close in time with administration of the secondary agent, including within about 3 hours, 2 hours, 1 hour, 0.5 hours, 0.25 hours, and 0.1 hours.

In some embodiments, administration of an LNP-formulated mRNA encoding a therapeutic polypeptide is performed following administration of another therapy (e.g., a secondary agent). In some embodiments, the additional therapy (e.g., secondary agent) is administered prior to and within about 144 hours, 120 hours, 96 hours, 72 hours, 24 hours, 18 hours, 12 hours, or 6 hours of administration of the polynucleotide (e.g., mRNA). In some embodiments, the additional therapy (e.g., secondary agent) is administered about 96-144, 72-120, 24-72, 12-24, or 6-24 hours prior to administration of an LNP-formulated mRNA.

Subjects who have been administered one or more secondary agents 2 or more hours prior to administration of an LNP-formulated mRNA may be referred to as having been pre-medicated with such agent(s). Subjects who have been administered one or more secondary agents within about 1-3, 0.5-2, 0.1-1 hours of administration of an LNP-formulated mRNA may be referred to as having been co-medicated with such agent(s).

In some embodiments, the secondary agent(s) is administered continuously to the subject, on an as needed basis or on a regular schedule (e.g., every days, every two days, etc.).

In some embodiments, the secondary agent(s) is administered before or after administration of the LNP-formulated mRNA.

Accordingly, in some embodiments, the combination therapies provided herein involve administrating to a subject to in need thereof a secondary agent that is a pain reliever (e.g., a non-steroidal anti-inflammatory drugs (NSAIDs)), a medication for epileptic seizures, a medication that is an anti-coagulant agent, a medication that is an anti-platelet agent, a medication that is an anti-thrombotic agent, a medication that is a fibrinolytic agent, a medication that is an antihistamine, a medication that is an antibiotic, and/or a medication that is a steroid. or other therapy aimed at alleviating or controlling symptoms associated with the disease or disorder and/or administration of the polynucleotide or composition thereof disclosed herein.

In some embodiments, administration of an LNP-formulated mRNA is performed with pre-medication and/or co-medication with an NSAID. NSAIDs include but are not limited to naproxen sodium, diclofenae, sulindac, oxaprozin, difluinisal, aspirin, piroxicam, indomethocin, etodolac, ibuprofen, fenoprofen, ketoprofen, mefenamic acid, nabumetone, tolmetin sodium, and ketorolac tromethamine.

In some embodiments, administration of an LNP-formulated mRNA is performed with pre-medication and/or co-medication with a corticosteroid, such as but not limited to dexamethasone.

In some embodiments, administration of an LNP-formulated mRNA is performed with pre-medication and/or co-medication with an antihistamine, such as but not limited H1 receptor antagonists and H1 receptor inverse agonists. Non-limiting examples of H1 receptor antagonists include but are not limited to brompheniramine, chlorpheniramine, dimenhydrinate, diphenhydramine, or doxylamine. Non-limiting examples of H1 receptor inverse agonists include but are not limited to pyrilamine, cetirizine, levocetirizine, and desloratadine.

In some embodiments, the administration of an LNP-formulated mRNA encoding a therapeutic polypeptide and one or more additional therapies in accordance with the methods presented herein have an additive effect relative the administration of the polynucleotide or said one or more additional therapies alone. In some embodiments, the administration of an LNP-formulated mRNA and one or more additional therapies in accordance with the methods presented herein have a synergistic effect relative to the administration of the polynucleotide or said one or more additional therapies alone.

In some embodiments, the combination of an LNP-formulated mRNA and one or more additional therapies is administered to a subject in the same pharmaceutical composition. Alternatively, an LNP-formulated mRNA and one or more additional therapies is administered concurrently to a subject in separate pharmaceutical compositions. In some embodiments, an LNP-formulated mRNA and one or more additional therapies is administered sequentially to a subject in separate pharmaceutical compositions. In some embodiments, an LNP-formulated mRNA and one or more additional therapies is administered to a subject by the same or different routes of administration.

### Clinical Objectives and Endpoints

In some embodiments, maintenance of a desired effect is determined by analyzing a biological sample (e.g., biopsy) at least once during a dosing interval. In some embodiments, maintenance of a desired effect is determined by analyzing a biological sample (e.g., biopsy) at regular intervals during a dosing interval. In some embodiments, maintenance of a desired effect is determined by analyzing a biological sample (e.g., biopsy) before a subsequent dose is administered.

In some embodiments, dosing occurs until a positive therapeutic outcome is achieved. In some embodiments, dosing of a composition comprising an LNP-formulated mRNA encoding a therapeutic polypeptide will occur indefinitely, or until a positive therapeutic outcome is achieved. In some embodiments, the dosing interval remains consistent. In some embodiments, the dosing interval changes as needed based on a clinician's assessment.

In some aspects, efficacy of an LNP-formulated mRNA for treating a disease or disorder that would benefit from increased expression of a therapeutic polypeptide is assessed by determining the effects of the polynucleotide on reduction of a biomarker of the therapeutic polypeptide. The efficacy of an LNP-formulated mRNA for treating a disease or disorder that would benefit from increased expression of a therapeutic polypeptide may also be assessed by: (i) determining the effect on levels of one or more biomarker of the therapeutic polypeptides; (ii) determining the effects on enzyme activity in cultured fibroblasts and lymphocytes from subjects with the disease or disorder that would benefit from increased expression of a therapeutic polypeptide, (iii) evaluating the safety profile of the LNP-formulated mRNA; (iv) evaluating compliance with treatment with the LNP-formulated mRNA; and (v) determining the LNP-formulated mRNA's plasma exposure over time.

A primary clinical endpoint for efficacy of an LNP-formulated mRNAfor treating a disease or disorder that would benefit from increased expression of a therapeutic polypeptide includes a reduction in a biomarker of the therapeutic polypeptide. Other clinical endpoints for the efficacy of an LNP-formulated mRNA for treating a disease or disorder that would benefit from increased expression of a therapeutic polypeptide may include a reduction in one or more biomarker of the therapeutic polypeptide and pharmacokinetic parameters, e.g., time to maximum plasma concentration (Tₘₐₓ), Cₘₐₓ, AUC, terminal elimination half-life (t_{1/2}) based on an LNP-formulated mRNA's plasma concentrations as assessed by a validated bioanalytical method.

### Therapeutic Polypeptides

In some aspects, the present disclosure provides mRNAs comprising an open reading frame (ORF) encoding polypeptides of interest (e.g., therapeutic proteins). In some embodiments, the polypeptide of interest is a therapeutic polypeptide. In some embodiments, the disclosure provides method of generating an mRNA comprising an ORF that encodes a polypeptide of interest (e.g., a therapeutic polypeptide), typically a protein or peptide having therapeutic properties for use in a subject. The polypeptides of interest can be essentially any protein or polypeptide that can be encoded by an mRNA.

In some embodiments, an mRNA of the disclosure encodes a polypeptide of interest that is a full-length protein. In some embodiments, an mRNA of the disclosure encodes a polypeptide of interest that is a functional fragment of a full-length protein (e.g., a fragment of the full-length protein that includes one or more functional domains such that the functional activity of the full-length protein is retained). In some embodiments, an mRNA of the disclosure encodes a polypeptide of interest that is not naturally occurring. In some embodiments, an mRNA of the disclosure encodes a polypeptide of interest that is a modified protein comprised of one or more heterologous domains (e.g., a protein that is a fusion protein comprised of one or more domains that do not naturally occur in the protein such that the function of the protein is altered).

Exemplary types of proteins (e.g., infectious disease antigens, tumor cell antigens, soluble effector molecules, antibodies, enzymes, recruitment factors, transcription factors, membrane bound receptors or ligands) that are encoded by an mRNA of the disclosure are described in detail in the following subsections.

### Naturally Occurring Targets

In some embodiments, an mRNA of the disclosure encodes a polypeptide of interest that is a naturally occurring target. In some embodiments, an mRNA encodes a polypeptide of interest that when expressed, modulates a naturally occurring target (e.g., up- or down-regulates the activity of a naturally occurring target). In some embodiments, a naturally occurring target is a soluble protein that is secreted by a cell. In some embodiments, a naturally occurring target is a protein that is retained within a cell (e.g., an intracellular protein). In some embodiments, a naturally occurring target is a membrane-bound or transmembrane protein. Non-limiting examples of naturally occurring targets include soluble proteins (e.g., chemokines, cytokines, growth factors, antibodies, enzymes), intracellular proteins (e.g., intracellular signaling proteins, transcription factors, enzymes, structural proteins) and membrane-bound or transmembrane proteins (e.g., receptors, adhesion molecules, enzymes).

### Naturally Occurring Soluble Targets

In some embodiments, an mRNA encodes a polypeptide of interest that modulates the activity of a naturally occurring soluble target, for example by encoding the soluble target itself or by modulating the expression (e.g., transcription or translation) of the soluble target. Non-limiting examples of naturally occurring soluble targets include cytokines, chemokines, growth factors, enzymes, and antibodies.

In some embodiments, an mRNA encoding a polypeptide of interest stimulates (e.g., upregulates, enhances) the activation or activity of a cell type, for example in situations where stimulation of an immune response is desirable, such as in cancer therapy or treatment of an infectious disease (e.g., a viral, bacterial, fungal, protozoal or parasitic infection). In another embodiment, an mRNA encoding a polypeptide of interest inhibits (e.g., downregulates, reduces) the activation or activity of a cell, for example in situations where inhibition of an immune response is desirable, such as in autoimmune diseases, allergies and transplantation.

In some embodiments, an mRNA of the disclosure encodes a soluble target that is a cytokine or chemokine with desirable uses for stimulating or inhibiting immune responses, e.g., that is useful in treating cancer as described further below.

In some embodiments, an mRNA of the disclosure encodes a soluble target that is a cytokine that stimulates the activation or activity of a cell such as an immune cell.

In some embodiments, an mRNA of the disclosure encodes a chemokine or a chemokine receptor which is useful for stimulating the activation or activity of an immune cell. Chemokines have been demonstrated to control the trafficking of inflammatory cells (including granulocytes and monocytes monocytes), as well as regulating the movement of a wide variety of immune cells (including lymphocytes, natural killer cells and dendritic cells). Thus, chemokines are involved both in regulating inflammatory responses and immune responses. Moreover, chemokines have been shown to have effects on the proliferative and invasive properties of cancer cells (for a review of chemokines, see e.g., Mukaida, N. et al. (2014) Mediators of Inflammation, Article ID 170381, pg. 1-15).

In some embodiments, an mRNA of the disclosure encodes an inhibitory cytokine or an antagonist of a stimulatory cytokine which is useful for inhibiting immune responses.

In some embodiments, an mRNA of the disclosure encodes a soluble target that is an antibody. As used herein, the term “antibody” refers to a whole antibody comprising two light chain polypeptides and two heavy chain polypeptides, or an antigen-binding fragment thereof. In some embodiments, a soluble target is a monoclonal antibody (e.g., full length monoclonal antibody) that displays a single binding specificity and affinity for a particular epitope. In some embodiments, a soluble target is an antigen binding fragment of a monoclonal antibody that retains the ability to bind a target antigen. Such fragments include, e.g., a single chain antibody, a single chain Fv fragment (scFv), an Fd fragment, an Fab fragment, an Fab′ fragment, or an F(ab′)₂ fragment.

In some embodiments, an mRNA of the disclosure encodes an antibody that recognizes a tumor antigen, against which a protective or a therapeutic immune response is desired, e.g., antigens expressed by a tumor cell. In some embodiments, a suitable antigen includes tumor associated antigens for the prevention or treatment of cancers.

In some embodiments, an mRNA of the disclosure encodes an antibody that recognizes an infectious disease antigen, against which protective or therapeutic immune responses are desired, e.g., an antigen present on a pathogen or infectious agent. In some embodiments, a suitable antigen includes an infectious disease associated antigen for the prevention or treatment of an infectious disease. Methods for identification of antigens on infectious disease agents that comprise protective epitopes (e.g., epitopes that when recognized by an antibody enable neutralization or blocking of infection caused by an infectious disease agent) are described in the art as detailed by Sharon, J. et al. (2013) Immunology 142:1-23. In some embodiments, an infectious disease antigen is present on a virus or on a bacterial cell.

In some embodiments, an mRNA of the disclosure encodes a soluble target that is an enzyme with desirable uses for modulating metabolism or growth in a subject. In some embodiments, an enzyme is administered to replace an endogenous enzyme that is absent or dysfunctional as described in Brady, R. et al, (2004) Lancet Neurol. 3:752. In some embodiments, an enzyme is used to treat a metabolic storage disease. A metabolic storage disease results from the systemic accumulation of metabolites due to the absence or dysfunction of an endogenous enzyme. Such metabolites include lipids, glycoproteins, and mucopolysaccharides. In some embodiments, an enzyme is used to reduce or eliminate the accumulation of monosaccharides, polysaccharides, glycoproteins, glycopeptides, glycolipids or lipids due to a metabolic storage disease.

### Naturally Occurring Intracellular Targets

In some embodiments, an mRNA of the disclosure encodes a polypeptide of interest that modulates the activity of a naturally occurring intracellular target, for example by encoding the intracellular target itself or by modulating the expression (e.g., transcription or translation) of the intracellular target in a cell. Non-limiting examples of naturally-occurring intracellular targets include transcription factors and cell signaling cascade molecules, including enzymes, that modulate cell growth, differentiation and communication. Additional examples include intracellular targets that regulate cell metabolism.

In some embodiments, an mRNA of the disclosure encodes an intracellular target that is a protein that is used to treat a metabolic disease or disorder.

In some embodiments, an mRNA of the disclosure encodes a polypeptide of interest that is a fully-functional mitochondrial protein (e.g., wild-type). In some embodiments, an mRNA of the disclosure encodes a mitochondrial protein encoded by mitochondrial DNA (e.g., a mitochondrial-encoded mitochondrial protein). In some embodiments, an mRNA of the disclosure encodes a mitochondrial protein encoded by nuclear DNA (e.g., a nuclear-encoded mitochondrial protein). In some embodiments, an mRNA of the disclosure is used to treat a mitochondrial disease resulting from a mutation in a mitochondrial protein. In some embodiments, translation of an mRNA encoding a mitochondrial protein provides sufficient quantity and/or activity of the protein to ameliorate a mitochondrial disease. In some embodiments, an mRNA encodes a polypeptide of interest that is a mitochondrial protein described in the MitoCarta2.0 mitochondrial protein inventory.

### Naturally Occurring Membrane Bound/Transmembrane Targets

In some embodiments, an mRNA of the disclosure encodes a polypeptide of interest that modulates the activity of a naturally-occurring membrane-bound/transmembrane target, for example by encoding the membrane-bound/transmembrane target itself or by modulating the expression (e.g., transcription or translation) of the membrane-bound/transmembrane target. Non-limiting examples of naturally-occurring membrane-bound/transmembrane targets include Cell surface receptors, growth factor receptors, costimulatory molecules, immune checkpoint molecules, homing receptors and HLA molecules.

### Modified Targets

In some embodiments, an mRNA of the disclosure encodes a polypeptide of interest that is a modified polypeptide. In some embodiments, an mRNA of the disclosure encodes a polypeptide of interest that modulates a modified target (e.g., up- or down-regulates the activity of a non-naturally-occurring target). Typically, an mRNA of the disclosure encodes a modified target. Alternatively, if a cell expresses a modified target, an mRNA-encoded polypeptide functions to modulate the activity of the modified target in the cell. In some embodiments, a non-naturally occurring target is a full-length target, such as a full-length modified protein. In some embodiments, a non-naturally occurring target is a fragment or portion of a non-naturally-occurring target, such as a fragment or portion of a modified protein. In some embodiments, an mRNA-encoded polypeptide when expressed acts in an autocrine fashion to modulate a modified target, i.e., exerts an effect directly on the cell into which the mRNA is delivered. Additionally or alternatively, an mRNA-encoded polypeptide when expressed acts in a paracrine fashion to modulates a modified target, i.e., exerts an effect indirectly on a cell other than the cell into which the mRNA is delivered (e.g., delivery of the mRNA into one type of cell results in secretion of a molecule that exerts effects on another type of cell, such as bystander cells). Non-limiting examples of modified proteins include modified soluble proteins (e.g., secreted proteins), modified intracellular proteins (e.g., intracellular signaling proteins, transcription factors) and modified membrane-bound or transmembrane proteins (e.g., receptors).

### Modified Soluble Targets

In some embodiments, an mRNA of the disclosure encodes a polypeptide of interest that modulates a modified soluble target (e.g., up- or down-regulates the activity of a non-naturally-occurring soluble target). In some embodiments, an mRNA of the disclosure encodes a polypeptide of interest that is a modified soluble target. In some embodiments, a modified soluble target is a soluble protein that has been modified to alter (e.g., increase or decrease) the half-life (e.g., serum half-life) of the protein. Modified soluble proteins with altered half-life include modified cytokines and chemokines. In some embodiments, a modified soluble target is a soluble protein that has been modified to incorporate a tether such that the soluble protein becomes tethered to a cell surface. Modified soluble proteins incorporating a tether include tethered cytokines and chemokines.

### Modified Intracellular Targets

In some embodiments, an mRNA of the disclosure encodes a polypeptide of interest that modulates a modified intracellular target (e.g., up- or down-regulates the activity of a non-naturally-occurring intracellular target). In some embodiments, an mRNA of the disclosure encodes polypeptide of interest that is a modified intracellular target. In some embodiments, a modified intracellular target is a constitutively active mutant of an intracellular protein, such as a constitutively active transcription factor or intracellular signaling molecule. In some embodiments, a modified intracellular target is a dominant negative mutant of an intracellular protein, such as a dominant negative mutant of a transcription factor or intracellular signaling molecule. In some embodiments, a modified intracellular target is an altered (e.g., mutated) enzyme, such as a mutant enzyme with increased or decreased activity within an intracellular signaling cascade.

### Modified Membrane Bound/Transmembrane Targets

In some embodiments, an mRNA of the disclosure encodes a polypeptide of interest that modulates a modified membrane-bound/transmembrane target (e.g., up- or down-regulates the activity of a non-naturally-occurring membrane-bound/transmembrane target). In some embodiments, an mRNA of the disclosure encodes a polypeptide of interest that is a modified membrane-bound/transmembrane target. In some embodiments, a modified membrane-bound/transmembrane target is a constitutively active mutant of a membrane-bound/transmembrane protein, such as a constitutively active cell surface receptor (i.e., activates intracellular signaling through the receptor without the need for ligand binding). In some embodiments, a modified membrane-bound/transmembrane target is a dominant negative mutant of a membrane-bound/transmembrane protein, such as a dominant negative mutant of a cell surface receptor. In some embodiments, a modified membrane-bound/transmembrane target is a molecule that inverts signaling of a cellular synapse (e.g., agonizes or antagonizes signaling of a receptor). In some embodiments, a modified membrane-bound/transmembrane target is a chimeric membrane-bound/transmembrane protein, such as a chimeric cell surface receptor.

As used herein, the term “chimeric antigen receptor (CAR)” refers to an artificial transmembrane protein receptor comprising an extracellular domain capable of binding to a predetermined CAR ligand or antigen, an intracellular segment comprising one or more cytoplasmic domains derived from signal transducing proteins different from the polypeptide from which the extracellular domain is derived, and a transmembrane domain.

### Antibodies Specific for Chikungunya Virus

The polynucleotides, constructs, and/or compositions of the present disclosure are useful for producing antibodies that bind to a chikungunya virus (CHIKV), e.g., to a CHIKV antigenic polypeptide.

In some embodiments the compositions and methods are useful for the prevention, treatment, or management of CHIKV infection, e.g., chikungunya fever. Some embodiments of the present disclosure provide RNA polynucleotides, e.g., mRNA, encoding an anti-CHIKV antibody, fragment, or variant thereof, which may be used to treat or prevent chikungunya fever. In some embodiments, one or more RNA polynucleotides have open reading frames (ORFs) encoding at least one anti-CHIKV antibody that binds specifically to a CHIKV antigenic polypeptide. In some embodiments, two or more RNA polynucleotides, e.g., two or more mRNAs, encode portions or fragments of an anti-CHIKV antibody. For example, one mRNA polynucleotide can have an ORF encoding a heavy chain of the anti-CHIKV antibody, and one mRNA polynucleotide can have an ORF encoding a light chain of the anti-CHIKV antibody, such that the two mRNAs in combination express the heavy and light chain polypeptides that together form the antibody, e.g., in a cell. In some embodiments, the mRNA polynucleotides described herein encode an antibody that neutralizes CHIKV.

An antibody is an immunoglobulin molecule capable of specific binding to a target through at least one antigen recognition site, located in the variable region of the immunoglobulin molecule.

As used herein, the term “antibody” encompasses not only intact (i.e., full-length) antibodies, but also antigen-binding fragments thereof (such as Fab, Fab′, F(ab′)2, Fv), single chain (scFv), mutants thereof, fusion proteins comprising an antibody portion, humanized antibodies, chimeric antibodies, diabodies, nanobodies, linear antibodies, single chain antibodies, multispecific antibodies (e.g., bispecific antibodies), single domain antibodies such as heavy-chain antibodies, and any other modified configuration of the immunoglobulin molecule that comprises an antigen recognition site of the required specificity, including glycosylation variants of antibodies, amino acid sequence variants of antibodies, and covalently modified antibodies. An antibody includes an antibody of any class, such as IgD, IgE, IgG, IgA, or IgM (or sub-class thereof), and the antibody need not be of any particular class.

In some embodiments, the antibodies described herein specifically bind to the corresponding target antigen or an epitope thereof. An antibody that “specifically binds” to an antigen or an epitope is a term well understood in the art. A molecule is said to exhibit “specific binding” if it reacts more frequently, more rapidly, with greater duration and/or with greater affinity with a particular target antigen than it does with alternative targets. An antibody “specifically binds” to a target antigen or epitope if it binds with greater affinity, avidity, more readily, and/or with greater duration than it binds to other substances.

In some embodiments, the mRNA polynucleotides described herein encode an antibody that binds to CHIKV. The mRNAs of the present disclosure can encode one or more polypeptides that form an antibody, or an antigen-binding portion thereof, that specifically binds to and neutralizes CHIKV. In one exemplary embodiment, mRNA polynucleotides described herein encode a heavy chain polypeptide of an antibody, a light chain polypeptide of an antibody, or heavy and light chain polypeptides of an antibody.

In some embodiments, the antibodies, or antigen binding fragments thereof, have a heavy chain polypeptide having an amino acid sequence sharing at least 80%, 81%, 82%, 83%, 84%, 85%, 86%, 87%, 88%, 89%, 90%, 91%, 92%, 93%, 94%, 95%, 96%, 97% 98%, or 99% identity with SEQ ID NO: 1. In some embodiments, the antibodies, or antigen binding fragments thereof, have a heavy chain polypeptide having an amino acid sequence that is identical to SEQ ID NO: 1. In some embodiments, the antibodies or antigen binding fragments thereof, have a heavy chain polypeptide having an amino acid sequence differing by up to 20 amino acids from SEQ ID NO: 1, e.g., differing by 1, 2, 3, 4, 5, 6, 7, 8, 9, 10, 11, 12, 13, 14, 15, 16, 17, 18, 19, or 20 amino acids from SEQ ID NO: 1.

In some embodiments, the antibodies, or antigen binding fragments thereof, have a light chain polypeptide having an amino acid sequence sharing at least 80%, 81%, 82%, 83%, 84%, 85%, 86%, 87%, 88%, 89%, 90%, 91%, 92%, 93%, 94%, 95%, 96%, 97% 98%, or 99% identity with SEQ ID NO: 3. In some embodiments, the antibodies, or antigen binding fragments thereof, have a light chain polypeptide having an amino acid sequence that is identical to SEQ ID NO: 3. In some embodiments, the antibodies or antigen binding fragments thereof, have a light chain polypeptide having an amino acid sequence differing by up to 20 amino acids from SEQ ID NO: 3, e.g., differing by 1, 2, 3, 4, 5, 6, 7, 8, 9, 10, 11, 12, 13, 14, 15, 16, 17, 18, 19, or 20 amino acids from SEQ ID NO: 3.

In some embodiments, the mRNA polynucleotides described herein encode one or more antibodies, or combinations of antibodies, selected from the group consisting of IgA, IgG, IgM, IgE, and IgD, that can bind specifically to CHIKV.

In some embodiments, the antibody described herein is a humanized antibody. Humanized antibodies refer to forms of non-human (e.g., murine) antibodies that are specific chimeric immunoglobulins, immunoglobulin chains, or antigen-binding fragments thereof that contain minimal sequence derived from non-human immunoglobulin.

In some embodiments, the heavy chain constant region used in the antibodies described herein may comprise mutations (e.g., amino acid residue substitutions) to enhance a desired characteristic of the antibody, for example, increasing the binding activity to the neonatal Fc receptor (FcRn) and thus the serum half-life of the antibodies. It was known that binding to FcRn is critical for maintaining antibody homeostasis and regulating the serum half-life of antibodies. One or more (e.g., 1, 2, 3, 4, 5, or more) mutations (e.g., amino acid residue substitutions) may be introduced into the constant region at suitable positions (e.g., in CH2 region) to enhance FcRn binding and enhance the half-life of the antibody. See, e.g., Dall'Acqua et al., J.B.C., 2006, 281:23514-23524; Robbie et al., Antimicrob. Agents Chemother, 2013, 57(12):6147; and Dall'Acqua et al., J. Immunol. 2002 169:5171-5180.

### Polynucleotides Encoding Anti-CHIKV

In some aspects, the polynucleotides disclosed herein are or function as a messenger RNA (mRNA). As used herein, the term “messenger RNA” (mRNA) refers to any polynucleotide which encodes at least one peptide or polypeptide of interest and which is capable of being translated to produce the encoded peptide polypeptide of interest in vitro, in vivo, in situ or ex vivo. The basic components of an mRNA molecule typically include at least one coding region, a 5′ untranslated region (UTR), a 3′ UTR, a 5′ cap, and a poly-A tail.

In some embodiments, the disclosure provides mRNAs for use in treating or preventing CHIKV infection in a subject. The mRNAs featured for use in the invention are administered to subjects and encode a human anti-CHIKV antibody in vivo. Accordingly, the invention relates to polynucleotides, e.g., mRNA, comprising an open reading frame of linked nucleosides encoding a human anti-CHIKV antibody polypeptide, functional fragments thereof, and fusion proteins. In some embodiments, the open reading frame is sequence-optimized. In particular embodiments, the invention provides sequence-optimized polynucleotides comprising nucleotides encoding a polypeptide sequence of a human anti-CHIKV antibody, or a portion or fragment thereof, e.g., nucleotides encoding a heavy chain or a light chain of an anti-CHIKV antibody.

In certain aspects, the invention provides polynucleotides (e.g., a RNA such as an mRNA) that comprise a nucleotide sequence (e.g., an ORF) encoding one or more anti-CHIKV antibody polypeptides. In some embodiments, the encoded anti-CHIKV antibody polypeptide of the invention can be selected from:

(i) a full length anti-CHIKV heavy chain polypeptide or a full length anti-CHIKV light chain polypeptide;

(ii) a functional fragment of an anti-CHIKV heavy chain or light chain polypeptide described herein (e.g., a truncated sequence shorter than the heavy or light chain; but still retaining CHIKV binding activity);

(iii) a variant (e.g., full length or truncated protein in which one or more amino acids have been replaced) with respect to a reference protein, e.g., a heavy chain (e.g., SEQ ID NO: 1) or light chain (e.g., SEQ ID NO: 3) of an anti-CHIKV antibody; or

(iv) a fusion protein comprising (i) a full length heavy chain (e.g., SEQ ID NO: 1), a functional fragment or a variant thereof, or (ii) a full length light chain (e.g., SEQ ID NO: 3), a functional fragment or a variant thereof; and (ii) a heterologous protein.

In certain embodiments, the encoded polypeptide is a mammalian anti-CHIKV antibody polypeptide, such as a human anti-CHIKV antibody polypeptide, a functional fragment or a variant thereof.

In some embodiments, one or more mRNA polynucleotide as described herein expresses an anti-CHIKV antibody in a mammalian cell, e.g., a human cell. In some embodiments, a first mRNA polynucleotide encodes a first polypeptide that is a heavy chain of an anti-CHIKV antibody, of a portion thereof (e.g., a heavy chain variable region), and a second mRNA polynucleotide encodes a second polypeptide that is a light chain of an anti-CHIKV antibody, or a portion thereof (e.g., a light chain variable region), such that the first and second polynucleotides express the heavy and light chains of an anti-CHIKV antibody in a mammalian cell, e.g., a human cell, and the heavy and light chains pair to form the anti-CHIKV antibody.

In some embodiments, the anti-CHIKV antibody expressed in the cell is secreted and can bind to CHIKV and/or neutralize CHIKV. Anti-CHIKV antibody protein expression levels and/or anti-CHIKV antibody activity, e.g., antigen binding activity and/or virus neutralization activity, can be measured according to methods known in the art. In some embodiments, the polynucleotide is introduced to the cells in vitro. In some embodiments, the polynucleotide is introduced to the cells in vivo by administration of the polynucleotide to a subject.

In some embodiments, the polynucleotides (e.g., a RNA, e.g., an mRNA) of the invention comprise a nucleotide sequence (e.g., an ORF) that encodes a heavy chain polypeptide, e.g., SEQ ID NO: 1. In some embodiments, the polynucleotides (e.g., a RNA, e.g., an mRNA) of the invention comprise a nucleotide sequence (e.g., an ORF) that encodes a light chain polypeptide, e.g., SEQ ID NO: 3.

In some embodiments, the polynucleotides (e.g., a RNA, e.g., an mRNA) of the invention comprise a nucleotide sequence (e.g., an ORF) that is identical to SEQ ID NO:2 or SEQ ID NO:4.

In some embodiments, the polynucleotides (e.g., an mRNA) described herein comprise a nucleotide sequence that is identical to SEQ ID NO: 5 or SEQ ID NO: 6

In some embodiments, the polynucleotide (e.g., a RNA, e.g., an mRNA) of the invention further comprises a 5′-UTR and a 3′UTR. In some embodiments, an mRNA described herein comprises a 5′ UTR comprising a nucleic acid sequence of SEQ ID NO: 13. In some embodiments, an mRNA described herein comprises a 3′ UTR comprising a nucleic acid sequence of SEQ ID NO: 14. In some embodiments, an mRNA described herein comprises an ORF encoding SEQ ID NO:2 or SEQ ID NO:4.

In a further embodiment, the polynucleotide (e.g., a RNA, e.g., an mRNA) comprises a 5′ terminal cap (e.g., Cap0, Cap1, ARCA, inosine, N1-methyl-guanosine, 2′-fluoro-guanosine, 7-deaza-guanosine, 8-oxo-guanosine, 2-amino-guanosine, LNA-guanosine, 2-azidoguanosine, Cap2, Cap4, 5′ methylG cap, or an analog thereof) and a poly-A-tail region (e.g., about 100 nucleotides in length). In some instances, the poly A tail is 50-150, 75-150, 85-150, 90-150, 90-120, 90-130, or 90-150 nucleotides in length. In some instances, the poly A tail is 100 nucleotides in length.

In some embodiments, the polynucleotide of the invention (e.g., a RNA, e.g., an mRNA) comprises a sequence-optimized nucleotide sequence (e.g., an ORF) encoding an anti-CHIKV antibody polypeptide (e.g., SEQ ID NOs:2 or 4), wherein the polynucleotide comprises at least one chemically modified nucleobase, e.g., N1-methylpseudouracil or 5-methoxyuracil. In certain embodiments, all uracils in the polynucleotide are N1-methylpseudouracils. In other embodiments, all uracils in the polynucleotide are 5-methoxyuracils. In some embodiments, the polynucleotide further comprises a miRNA binding site, e.g., a miRNA binding site that binds to miR-142 and/or a miRNA binding site that binds to miR-126.

In some embodiments, the polynucleotide (e.g., a RNA, e.g., a mRNA) disclosed herein is formulated with a delivery agent comprising, e.g., a compound having the Formula (I), (IIa) or (IIb), e.g., Compound 1; a compound having the Formula (III), e.g., Compounds 2; or any combination thereof. In some embodiments, the delivery agent comprises Compound 1, DSPC, Cholesterol, and Compound 2 or PEG-DMG, e.g., with a mole ratio of about 50:10:38:2. In some embodiments, the delivery agent comprises Compound 1, DSPC, Cholesterol, and Compound 2 or PEG-DMG, e.g., with a mole ratio in the range of about 30 to about 60 mol % Compound 1 (or related suitable amino lipid) (e.g., 30-40, 40-45, 45-50, 50-55 or 55-60 mol % Compound 1 (or related suitable amino lipid)), about 5 to about 20 mol % phospholipid (or related suitable phospholipid or “helper lipid”) (e.g., 5-10, 10-15, or 15-20 mol % phospholipid (or related suitable phospholipid or “helper lipid”)), about 20 to about 50 mol % cholesterol (or related sterol or “non-cationic” lipid) (e.g., about 20-30, 30-35, 35-40, 40-45, or 45-50 mol % cholesterol (or related sterol or “non-cationic” lipid)) and about 0.05 to about 10 mol % PEG lipid or Compound 2 (or other suitable PEG lipid) (e.g., 0.05-1, 1-2, 2-3, 3-4, 4-5, 5-7, or 7-10 mol % PEG lipid or Compound 2 (or other suitable PEG lipid)). An exemplary delivery agent can comprise mole ratios of, for example, 47.5:10.5:39.0:3.0 or 50:10:38:2. In certain instances, an exemplary delivery agent can comprise mole ratios of, for example, 47.5:10.5:39.0:3; 47.5:10:39.5:3; 47.5:11:39.5:2; 47.5:10.5:39.5:2.5; 47.5:11:39:2.5; 48.5:10:38.5:3; 48.5:10.5:39:2; 48.5:10.5:38.5:2.5; 48.5:10.5:39.5:1.5; 48.5:10.5:38.0:3; 47:10.5:39.5:3; 47:10:40.5:2.5; 47:11:40:2; 47:10.5:39.5:3; 48:10.5:38.5:3; 48:10:39.5:2.5; 48:11:39:2; or 48:10.5:38.5:3. In some embodiments, the delivery agent comprises Compound 1, DSPC, Cholesterol, and Compound 2 or PEG-DMG, e.g., with a mole ratio of about 47.5:10.5:39.0:3.0. In some embodiments, the delivery agent comprises Compound 1, DSPC, Cholesterol, and Compound 2, e.g., with a mole ratio of about 50:10:38:2.

In some embodiments, the polynucleotide of the disclosure is an mRNA that comprises a 5′-terminal cap (e.g., Cap 1), a 5′UTR (e.g., SEQ ID NO:13), a ORF sequence selected from the group consisting of SEQ ID NO: 2 and SEQ ID NO: 4, a 3′UTR (e.g., SEQ ID NO:14), and a poly A tail (e.g., about 100 nucleotides in length), wherein all uracils in the polynucleotide are N1-methylpseudouracils. In some embodiments, the delivery agent comprises Compound 1 as the ionizable lipid and PEG-DMG or Compound 2 as the PEG lipid, or any combinations thereof (e.g., Compound 1 and Compound 2).

In some embodiments, the disclosure provides an mRNA comprising an ORF sequence set forth by SEQ ID NO: 2 and an mRNA comprising an ORF sequence set forth by SEQ ID NO: 4, wherein the mRNAs are co-formulated in the same lipid nanoparticle. In some embodiments, the mRNA comprising an ORF with a nucleotide sequence set forth by SEQ ID NO: 2 and an mRNA comprising an ORF with a nucleotide sequence set forth by SEQ ID NO: 4 are co-formulated in a lipid nanoparticle at a ratio of about 0.5:1, 1:1, 1.5:1, 2:1, 2.5:1, 3:1, 3.5:1, or 4:1. In some embodiments, the mRNA comprising an ORF with a nucleotide sequence set forth by SEQ ID NO: 2 and an mRNA comprising an ORF with a nucleotide sequence set forth by SEQ ID NO: 4 are co-formulated in a lipid nanoparticle at a ratio of about 2:1.

mRNA Construct Components

In some embodiments, the disclosure provides a lipid nanoparticle comprising at least one mRNA, for use in the methods described herein.

An mRNA may be a naturally or non-naturally occurring mRNA. An mRNA may include one or more modified nucleobases, nucleosides, or nucleotides, as described below, in which case it may be referred to as a “modified mRNA” or “mmRNA.” As described herein “nucleoside” is defined as a compound containing a sugar molecule (e.g., a pentose or ribose) or derivative thereof in combination with an organic base (e.g., a purine or pyrimidine) or a derivative thereof (also referred to herein as “nucleobase”). As described herein, “nucleotide” is defined as a nucleoside including a phosphate group.

An mRNA may include a 5′ untranslated region (5′-UTR), a 3′ untranslated region (3′-UTR), and/or a coding region (e.g., an open reading frame). Exemplary 5′UTR and 3′UTRs for use in the constructs are shown in Table 1. An mRNA may include any suitable number of base pairs, including tens (e.g., 10, 20, 30, 40, 50, 60, 70, 80, 90 or 100), hundreds (e.g., 200, 300, 400, 500, 600, 700, 800, or 900) or thousands (e.g., 1000, 2000, 3000, 4000, 5000, 6000, 7000, 8000, 9000, 10,000) of base pairs. Any number (e.g., all, some, or none) of nucleobases, nucleosides, or nucleotides may be an analog of a canonical species, substituted, modified, or otherwise non-naturally occurring. In certain embodiments, all of a particular nucleobase type may be modified.

TABLE 1
Exemplary 5′UTR and 3′UTRs

| | | SEQ | |
|---|---|---|---|
| | | ID | |
| Identifier | Name | NO | Nucleotide sequence |
| 5′UTR | 5′UTR v1.1 | 13 | GGGAAAUAAGAGAGAAAAGAAGA |
| | | | GUAAGAAGAAAUAUAAGACCCCG |
| | | | GCGCCGCCACC |
| 5′UTR | 5′UTR v1.0 | 15 | GGGAAAUAAGAGAGAAAAGAAGA |
| | | | GUAAGAAGAAAUAUAAGAGCCAC |
| | | | C |
| 3′UTR | 3′UTR v1.1 | 14 | UGAUAAUAGGCUGGAGCCUCGGU |
| | | | GGCCUAGCUUCUUGCCCCUUGGG |
| | | | CCUCCCCCCAGCCCCUCCUCCCC |
| | | | UUCCUGCACCCGUACCCCCGUGG |
| | | | UCUUUGAAUAAAGUCUGAGUGGG |
| | | | CGGC |
| 3′UTR | 3′UTR v1.0 | 16 | UGAUAAUAGGCUGGAGCCUCGGU |
| | | | GGCCAUGCUUCUUGCCCCUUGGG |
| | | | CCUCCCCCCAGCCCCUCCUCCCC |
| | | | UUCCUGCACCCGUACCCCCGUGG |
| | | | UCUUUGAAUAAAGUCUGAGUGGG |
| | | | CGGC |

In some embodiments, an mRNA as described herein may include a 5′ cap structure, a chain terminating nucleotide, optionally a Kozak sequence (also known as a Kozak consensus sequence), a stem loop, a polyA sequence, and/or a polyadenylation signal.

A 5′ cap structure or cap species is a compound including two nucleoside moieties joined by a linker and may be selected from a naturally occurring cap, a non-naturally occurring cap or cap analog, or an anti-reverse cap analog (ARCA). A cap species may include one or more modified nucleosides and/or linker moieties. For example, a natural mRNA cap may include a guanine nucleotide and a guanine (G) nucleotide methylated at the 7 position joined by a triphosphate linkage at their 5′ positions, e.g., m⁷G(5′)ppp(5′)G, commonly written as m⁷GpppG. A cap species may also be an anti-reverse cap analog. A non-limiting list of possible cap species includes m⁷GpppG, m⁷Gpppm⁷G, m⁷3′dGpppG, m₂^{7,O3′}GpppG, m₂^{7,O3′}GppppG, m^{7,O2′}GpppG, m⁷Gpppm⁷G, m⁷3′dGpppG, m₂^{7,O3′}GpppG, m₂^{7,O3′}GppppG, and m₂^{7,O2′}GppppG.

An mRNA may instead or additionally include a chain terminating nucleoside. For example, a chain terminating nucleoside may include those nucleosides deoxygenated at the 2′ and/or 3′ positions of their sugar group. Such species may include 3′-deoxyadenosine (cordycepin), 3′-deoxyuridine, 3′-deoxycytosine, 3′-deoxyguanosine, 3′-deoxythymine, and 2′,3′-dideoxynucleosides, such as 2′,3′-dideoxyadenosine, 2′,3′-dideoxyuridine, 2′,3′-dideoxycytosine, 2′,3′-dideoxyguanosine, and 2′,3′-dideoxythymine. In some embodiments, incorporation of a chain terminating nucleotide into an mRNA, for example at the 3′-terminus, may result in stabilization of the mRNA, as described, for example, in International Patent Publication No. WO 2013/103659.

An mRNA may instead or additionally include a stem loop, such as a histone stem loop. A stem loop may include 2, 3, 4, 5, 6, 7, 8, or more nucleotide base pairs. For example, a stem loop may include 4, 5, 6, 7, or 8 nucleotide base pairs. A stem loop may be located in any region of an mRNA. For example, a stem loop may be located in, before, or after an untranslated region (a 5′ untranslated region or a 3′ untranslated region), a coding region, or a polyA sequence or tail. In some embodiments, a stem loop may affect one or more function(s) of an mRNA, such as initiation of translation, translation efficiency, and/or transcriptional termination.

An mRNA may instead or additionally include a polyA sequence and/or polyadenylation signal. A polyA sequence may be comprised entirely or mostly of adenine nucleotides or analogs or derivatives thereof. A polyA sequence may be a tail located adjacent to a 3′ untranslated region of an mRNA. In some embodiments, a polyA sequence may affect the nuclear export, translation, and/or stability of an mRNA.

An mRNA may instead or additionally include a microRNA binding site.

Modified mRNAs

In some embodiments, an mRNA of the disclosure comprises one or more modified nucleobases, nucleosides, or nucleotides (termed “modified mRNAs” or “mmRNAs”). In some embodiments, modified mRNAs may have useful properties, including enhanced stability, intracellular retention, enhanced translation, and/or the lack of a substantial induction of the innate immune response of a cell into which the mRNA is introduced, as compared to a reference unmodified mRNA. Therefore, use of modified mRNAs may enhance the efficiency of protein production, intracellular retention of nucleic acids, as well as possess reduced immunogenicity.

In some embodiments, an mRNA includes one or more (e.g., 1, 2, 3 or 4) different modified nucleobases, nucleosides, or nucleotides. In some embodiments, an mRNA includes one or more (e.g., 1, 2, 3, 4, 5, 6, 7, 8, 9, 10, 20, 30, 40, 50, 60, 70, 80, 90, 100, or more) different modified nucleobases, nucleosides, or nucleotides. In some embodiments, the modified mRNA may have reduced degradation in a cell into which the mRNA is introduced, relative to a corresponding unmodified mRNA.

In some embodiments, the modified nucleobase is a modified uracil. Exemplary nucleobases and nucleosides having a modified uracil include pseudouridine (ψ), pyridin-4-one ribonucleoside, 5-aza-uridine, 6-aza-uridine, 2-thio-5-aza-uridine, 2-thio-uridine (s²U), 4-thio-uridine (s⁴U), 4-thio-pseudouridine, 2-thio-pseudouridine, 5-hydroxy-uridine (ho⁵U), 5-aminoallyl-uridine, 5-halo-uridine (e.g., 5-iodo-uridineor 5-bromo-uridine), 3-methyl-uridine (m³U), 5-methoxy-uridine (mo⁵U), uridine 5-oxyacetic acid (cmo⁵U), uridine 5-oxyacetic acid methyl ester (mcmo⁵U), 5-carboxymethyl-uridine (cm⁵U), 1-carboxymethyl-pseudouridine, 5-carboxyhydroxymethyl-uridine (chm⁵U), 5-carboxyhydroxymethyl-uridine methyl ester (mchm⁵U), 5-methoxycarbonylmethyl-uridine (mcm⁵U), 5-methoxycarbonylmethyl-2-thio-uridine (mcm⁵s²U), 5-aminomethyl-2-thio-uridine (nm⁵s²U), 5-methylaminomethyl-uridine (mnm⁵U), 5-methylaminomethyl-2-thio-uridine (mnm⁵s²U), 5-methylaminomethyl-2-seleno-uridine (mnm⁵se²U), 5-carbamoylmethyl-uridine (ncm⁵U), 5-carboxymethylaminomethyl-uridine (cmnm⁵U), 5-carboxymethylaminomethyl-2-thio-uridine (cmnm⁵s²U), 5-propynyl-uridine, 1-propynyl-pseudouridine, 5-taurinomethyl-uridine (τm⁵U), 1-taurinomethyl-pseudouridine, 5-taurinomethyl-2-thio-uridine(τm⁵s²U), 1-taurinomethyl-4-thio-pseudouridine, 5-methyl-uridine (m⁵U, i.e., having the nucleobase deoxythymine), 1-methyl-pseudouridine (m¹ψ), 5-methyl-2-thio-uridine (m⁵s²U), 1-methyl-4-thio-pseudouridine (m¹s⁴ψ), 4-thio-1-methyl-pseudouridine, 3-methyl-pseudouridine (m³ψ), 2-thio-1-methyl-pseudouridine, 1-methyl-1-deaza-pseudouridine, 2-thio-1-methyl-1-deaza-pseudouridine, dihydrouridine (D), dihydropseudouridine, 5,6-dihydrouridine, 5-methyl-dihydrouridine (m⁵D), 2-thio-dihydrouridine, 2-thio-dihydropseudouridine, 2-methoxy-uridine, 2-methoxy-4-thio-uridine, 4-methoxy-pseudouridine, 4-methoxy-2-thio-pseudouridine, N1-methyl-pseudouridine, 3-(3-amino-3-carboxypropyl)uridine (acp³U), 1-methyl-3-(3-amino-3-carboxypropyl)pseudouridine (acp³ ψ), 5-(isopentenylaminomethyl)uridine (inm⁵U), 5-(isopentenylaminomethyl)-2-thio-uridine (inm⁵s²U), α-thio-uridine, 2′-O-methyl-uridine (Um), 5,2′-O-dimethyl-uridine (m⁵Um), 2′-O-methyl-pseudouridine (ψm), 2-thio-2′-O-methyl-uridine (s²Um), 5-methoxycarbonylmethyl-2′-O-methyl-uridine (mcm⁵Um), 5-carbamoylmethyl-2′-O-methyl-uridine (ncm⁵Um), 5-carboxymethylaminomethyl-2′-O-methyl-uridine (cmnm⁵Um), 3,2′-O-dimethyl-uridine (m³Um), and 5-(isopentenylaminomethyl)-2′-O-methyl-uridine (inm⁵Um), 1-thio-uridine, deoxythymidine, 2′-F-ara-uridine, 2′-F-uridine, 2′-OH-ara-uridine, 5-(2-carbomethoxyvinyl) uridine, and 5-[3-(1-E-propenylamino)]uridine.

In some embodiments, the modified nucleobase is a modified cytosine. Exemplary nucleobases and nucleosides having a modified cytosine include 5-aza-cytidine, 6-aza-cytidine, pseudoisocytidine, 3-methyl-cytidine (m³C), N4-acetyl-cytidine (ac⁴C), 5-formyl-cytidine (f⁵C), N4-methyl-cytidine (m⁴C), 5-methyl-cytidine (m⁵C), 5-halo-cytidine (e.g., 5-iodo-cytidine), 5-hydroxymethyl-cytidine (hm⁵C), 1-methyl-pseudoisocytidine, pyrrolo-cytidine, pyrrolo-pseudoisocytidine, 2-thio-cytidine (s²C), 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-1-methyl-pseudoisocytidine, lysidine (k₂C), a-thio-cytidine, 2′-O-methyl-cytidine (Cm), 5,2′-O-dimethyl-cytidine (m⁵Cm), N4-acetyl-2′-O-methyl-cytidine (ac⁴Cm), N4,2′-O-dimethyl-cytidine (m⁴Cm), 5-formyl-2′-O-methyl-cytidine (f⁵Cm), N4,N4,2′-O-trimethyl-cytidine (m⁴₂Cm), 1-thio-cytidine, 2′-F-ara-cytidine, 2′-F-cytidine, and 2′-OH-ara-cytidine.

In some embodiments, the modified nucleobase is a modified adenine. Exemplary nucleobases and nucleosides having a modified adenine include α-thio-adenosine, 2-amino-purine, 2,6-diaminopurine, 2-amino-6-halo-purine (e.g., 2-amino-6-chloro-purine), 6-halo-purine (e.g., 6-chloro-purine), 2-amino-6-methyl-purine, 8-azido-adenosine, 7-deaza-adenine, 7-deaza-8-aza-adenine, 7-deaza-2-amino-purine, 7-deaza-8-aza-2-amino-purine, 7-deaza-2,6-diaminopurine, 7-deaza-8-aza-2,6-diaminopurine, 1-methyl-adenosine (m¹A), 2-methyl-adenine (m²A), N6-methyl-adenosine (m⁶A), 2-methylthio-N6-methyl-adenosine (ms²m⁶A), N6-isopentenyl-adenosine (i⁶A), 2-methylthio-N6-isopentenyl-adenosine (ms²i⁶A), N6-(cis-hydroxyisopentenyl)adenosine (io⁶A), 2-methylthio-N6-(cis-hydroxyisopentenyl)adenosine (ms²io⁶A), N6-glycinylcarbamoyl-adenosine (g⁶A), N6-threonylcarbamoyl-adenosine (t⁶A), N6-methyl-N6-threonylcarbamoyl-adenosine (m⁶t⁶A), 2-methylthio-N6-threonylcarbamoyl-adenosine (ms²g⁶A), N6,N6-dimethyl-adenosine (m⁶2A), N6-hydroxynorvalylcarbamoyl-adenosine (hn⁶A), 2-methylthio-N6-hydroxynorvalylcarbamoyl-adenosine (ms²hn⁶A), N6-acetyl-adenosine (ac⁶A), 7-methyl-adenine, 2-methylthio-adenine, 2-methoxy-adenine, α-thio-adenosine, 2′-O-methyl-adenosine (Am), N6,2′-O-dimethyl-adenosine (m⁶Am), N6,N6,2′-O-trimethyl-adenosine (m⁶₂Am), 1,2′-O-dimethyl-adenosine (m¹Am), 2′-O-ribosyladenosine (phosphate) (Ar(p)), 2-amino-N6-methyl-purine, 1-thio-adenosine, 8-azido-adenosine, 2′-F-ara-adenosine, 2′-F-adenosine, 2′-OH-ara-adenosine, and N6-(19-amino-pentaoxanonadecyl)-adenosine.

In some embodiments, the modified nucleobase is a modified guanine. Exemplary nucleobases and nucleosides having a modified guanine include a-thio-guanosine, inosine (I), 1-methyl-inosine (m¹I), wyosine (imG), methylwyosine (mimG), 4-demethyl-wyosine (imG-14), isowyosine (imG2), wybutosine (yW), peroxywybutosine (o₂yW), hydroxywybutosine (OhyW), undermodified hydroxywybutosine (OhyW*), 7-deaza-guanosine, queuosine (Q), epoxyqueuosine (oQ), galactosyl-queuosine (galQ), mannosyl-queuosine (manQ), 7-cyano-7-deaza-guanosine (preQo), 7-aminomethyl-7-deaza-guanosine (preQ₁), archaeosine (G⁺), 7-deaza-8-aza-guanosine, 6-thio-guanosine, 6-thio-7-deaza-guanosine, 6-thio-7-deaza-8-aza-guanosine, 7-methyl-guanosine (m⁷G), 6-thio-7-methyl-guanosine, 7-methyl-inosine, 6-methoxy-guanosine, 1-methyl-guanosine (m¹G), N2-methyl-guanosine (m²G), N2,N2-dimethyl-guanosine (m²₂G), N2,7-dimethyl-guanosine (m^{2,7}G), N2, N2,7-dimethyl-guanosine (m^{2,2,7}G), 8-oxo-guanosine, 7-methyl-8-oxo-guanosine, 1-methyl-6-thio-guanosine, N2-methyl-6-thio-guanosine, N2,N2-dimethyl-6-thio-guanosine, α-thio-guanosine, 2′-O-methyl-guanosine (Gm), N2-methyl-2′-O-methyl-guanosine (m²Gm), N2,N2-dimethyl-2′-O-methyl-guanosine (m²₂Gm), 1-methyl-2′-O-methyl-guanosine (m¹Gm), N2,7-dimethyl-2′-O-methyl-guanosine (m^{2,7}Gm), 2′-O-methyl-inosine (Im), 1,2′-O-dimethyl-inosine (m¹Im), 2′-O-ribosylguanosine (phosphate) (Gr(p)), 1-thio-guanosine, O6-methyl-guanosine, 2′-F-ara-guanosine, and 2′-F-guanosine.

In some embodiments, an mRNA of the disclosure includes a combination of one or more of the aforementioned modified nucleobases (e.g., a combination of 2, 3 or 4 of the aforementioned modified nucleobases).

In some embodiments, the modified nucleobase is pseudouridine (ψ), N1-methylpseudouridine (m¹ψ), 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine, or 2′-O-methyl uridine. In some embodiments, an mRNA of the disclosure includes a combination of one or more of the aforementioned modified nucleobases (e.g., a combination of 2, 3 or 4 of the aforementioned modified nucleobases).

In some embodiments, the modified nucleobase is a modified cytosine. Exemplary nucleobases and nucleosides having a modified cytosine include N4-acetyl-cytidine (ac⁴C), 5-methyl-cytidine (m⁵C), 5-halo-cytidine (e.g., 5-iodo-cytidine), 5-hydroxymethyl-cytidine (hm⁵C), 1-methyl-pseudoisocytidine, 2-thio-cytidine (s²C), 2-thio-5-methyl-cytidine. In some embodiments, an mRNA of the disclosure includes a combination of one or more of the aforementioned modified nucleobases (e.g., a combination of 2, 3 or 4 of the aforementioned modified nucleobases).

In some embodiments, the modified nucleobase is a modified adenine. Exemplary nucleobases and nucleosides having a modified adenine include 7-deaza-adenine, 1-methyl-adenosine (m¹A), 2-methyl-adenine (m²A), N6-methyl-adenosine (m⁶A). In some embodiments, an mRNA of the disclosure includes a combination of one or more of the aforementioned modified nucleobases (e.g., a combination of 2, 3 or 4 of the aforementioned modified nucleobases).

In some embodiments, the modified nucleobase is a modified guanine. Exemplary nucleobases and nucleosides having a modified guanine include inosine (I), 1-methyl-inosine (m¹I), wyosine (imG), methylwyosine (mimG), 7-deaza-guanosine, 7-cyano-7-deaza-guanosine (preQ₀), 7-aminomethyl-7-deaza-guanosine (preQ₁), 7-methyl-guanosine (m⁷G), 1-methyl-guanosine (m¹G), 8-oxo-guanosine, 7-methyl-8-oxo-guanosine. In some embodiments, an mRNA of the disclosure includes a combination of one or more of the aforementioned modified nucleobases (e.g., a combination of 2, 3 or 4 of the aforementioned modified nucleobases).

In some embodiments, the modified nucleobase is 1-methyl-pseudouridine (m¹ψ), 5-methoxy-uridine (mo⁵U), 5-methyl-cytidine (m⁵C), pseudouridine (ψ), α-thio-guanosine, or α-thio-adenosine. In some embodiments, an mRNA of the disclosure includes a combination of one or more of the aforementioned modified nucleobases (e.g., a combination of 2, 3 or 4 of the aforementioned modified nucleobases).

In some embodiments, the mRNA comprises pseudouridine (ψ). In some embodiments, the mRNA comprises pseudouridine (ψ) and 5-methyl-cytidine (m⁵C). In some embodiments, the mRNA comprises 1-methyl-pseudouridine (m¹ψ). In some embodiments, the mRNA comprises 1-methyl-pseudouridine (m¹ψ) and 5-methyl-cytidine (m⁵C). In some embodiments, the mRNA comprises 2-thiouridine (s²U). In some embodiments, the mRNA comprises 2-thiouridine and 5-methyl-cytidine (m⁵C). In some embodiments, the mRNA comprises 5-methoxy-uridine (mo⁵U). In some embodiments, the mRNA comprises 5-methoxy-uridine (mo⁵U) and 5-methyl-cytidine (m⁵C). In some embodiments, the mRNA comprises 2′-O-methyl uridine. In some embodiments, the mRNA comprises 2′-O-methyl uridine and 5-methyl-cytidine (m⁵C). In some embodiments, the mRNA comprises N6-methyl-adenosine (m⁶A). In some embodiments, the mRNA comprises N6-methyl-adenosine (m⁶A) and 5-methyl-cytidine (m⁵C).

In certain embodiments, an mRNA of the disclosure is uniformly modified (i.e., fully modified, modified through-out the entire sequence) for a particular modification. In some embodiments, an mRNA of the disclosure is modified wherein at least 95%, at least 96%, at least 97%, at least 98%, at least 99% or 100% of a specified nucleotide or nucleobase is modified. For example, an mRNA can be uniformly modified with 5-methyl-cytidine (m⁵C), meaning that all cytosine residues in the mRNA sequence are replaced with 5-methyl-cytidine (m⁵C). Similarly, mRNAs of the disclosure can be uniformly modified for any type of nucleoside residue present in the sequence by replacement with a modified residue such as those set forth above. In some embodiments, an mRNA of the disclosure is uniformly modified with 1-methyl pseudouridine (m¹ψ), meaning that all uridine residues in the mRNA sequence are replaced with 1-methyl pseudouridine (m¹ψ). In some embodiments, at least 95%, at least 96%, at least 97%, at least 98%, at least 99% or 100% of uridines are 1-methyl pseudouridine (m¹ψ).

In some embodiments, an mRNA of the disclosure may be modified in a coding region (e.g., an open reading frame encoding a polypeptide). In other embodiments, an mRNA may be modified in regions besides a coding region. For example, in some embodiments, a 5′-UTR and/or a 3′-UTR are provided, wherein either or both may independently contain one or more different nucleoside modifications. In such embodiments, nucleoside modifications may also be present in the coding region.

Examples of nucleoside modifications and combinations thereof that may be present in mmRNAs of the present disclosure include, but are not limited to, those described in PCT Patent Application Publications: WO2012045075, WO2014081507, WO2014093924, WO2014164253, and WO2014159813.

The mRNAs of the disclosure can include a combination of modifications to the sugar, the nucleobase, and/or the internucleoside linkage. These combinations can include any one or more modifications described herein.

Examples of modified nucleosides and modified nucleoside combinations thereof that may be present in mmRNAs of the present disclosure include, but are not limited to, those described in PCT Application Publications: WO 2012045075, WO2014081507, WO2014093924, WO2014164253 and WO2014159813.

The mmRNAs of the disclosure can include a combination of modifications to the sugar, the nucleobase, and/or the internucleoside linkage. These combinations can include any one or more modifications described herein.

In certain embodiments, the modified nucleosides may be partially or completely substituted for the natural nucleotides of the mRNAs of the disclosure. As a non-limiting example, the natural nucleotide uridine may be substituted with a modified nucleoside described herein. In another non-limiting example, the natural nucleoside uridine may be partially substituted (e.g., about 0.1%, 1%, 5%, 10%, 15%, 20%, 25%, 30%, 35%, 40%, 45%, 50%, 55%, 60%, 65%, 70%, 75%, 80%, 85%, 90%, 95% or 99.9% of the natural uridines) with at least one of the modified nucleoside disclosed herein.

The mRNAs of the present disclosure, or regions thereof, may be codon optimized. Codon optimization methods are known in the art and may be useful for a variety of purposes: matching codon frequencies in host organisms to ensure proper folding, bias GC content to increase mRNA stability or reduce secondary structures, minimize tandem repeat codons or base runs that may impair gene construction or expression, customize transcriptional and translational control regions, insert or remove proteins trafficking sequences, remove/add post translation modification sites in encoded proteins (e.g., glycosylation sites), add, remove or shuffle protein domains, insert or delete restriction sites, modify ribosome binding sites and mRNA degradation sites, adjust translation rates to allow the various domains of the protein to fold properly, or to reduce or eliminate problem secondary structures within the polynucleotide. Codon optimization tools, algorithms and services are known in the art; non-limiting examples include services from GeneArt (Life Technologies), DNA2.0 (Menlo Park, Calif.) and/or proprietary methods. In one embodiment, the mRNA sequence is optimized using optimization algorithms, e.g., to optimize expression in mammalian cells or enhance mRNA stability.

In certain embodiments, the present disclosure includes polynucleotides having at least 80%, at least 85%, at least 90%, at least 95%, at least 98%, or at least 99% sequence identity to any of the polynucleotide sequences described herein.

mRNAs of the present disclosure may be produced by means available in the art, including but not limited to in vitro transcription (IVT) and synthetic methods. Enzymatic (IVT), solid-phase, liquid-phase, combined synthetic methods, small region synthesis, and ligation methods may be utilized. In one embodiment, mRNAs are made using IVT enzymatic synthesis methods. Methods of making polynucleotides by IVT are known in the art and are described in International Application PCT/US2013/30062, the contents of which are incorporated herein by reference in their entirety. Accordingly, the present disclosure also includes polynucleotides, e.g., DNA, constructs and vectors that may be used to in vitro transcribe an mRNA described herein.

Non-natural modified nucleobases may be introduced into polynucleotides, e.g., mRNA, during synthesis or post-synthesis. In certain embodiments, modifications may be on internucleoside linkages, purine or pyrimidine bases, or sugar. In particular embodiments, the modification may be introduced at the terminal of a polynucleotide chain or anywhere else in the polynucleotide chain; with chemical synthesis or with a polymerase enzyme. Examples of modified nucleic acids and their synthesis are disclosed in PCT application No. PCT/US2012/058519. Synthesis of modified polynucleotides is also described in Verma and Eckstein, Annual Review of Biochemistry, vol. 76, 99-134 (1998).

Either enzymatic or chemical ligation methods may be used to conjugate polynucleotides or their regions with different functional moieties, such as targeting or delivery agents, fluorescent labels, liquids, nanoparticles, etc. Conjugates of polynucleotides and modified polynucleotides are reviewed in Goodchild, Bioconjugate Chemistry, vol. 1(3), 165-187 (1990).

### Adjusted Uracil Content

In some embodiments of the disclosure, an mRNA may have adjusted uracil content. In some embodiments, the uracil content of the open reading frame (ORF) of the polynucleotide encoding a therapeutic polypeptide relative to the theoretical minimum uracil content of a nucleotide sequence encoding the therapeutic polypeptide (% U_{TM}), is between about 100% and about 150. In some embodiments, the uracil content of the ORF is between about 105% and about 145%, about 105% and about 140%, about 110% and about 140%, about 110% and about 145%, about 115% and about 135%, about 105% and about 135%, about 110% and about 135%, about 115% and about 145%, or about 115% and about 140% of the theoretical minimum uracil content in the corresponding wild-type ORF (% U_{TM}). In other embodiments, the uracil content of the ORF is between about 117% and about 134% or between 118% and 132% of the % U_{TM}. In some embodiments, the uracil content of the ORF encoding a polypeptide is about 115%, about 120%, about 125%, about 130%, about 135%, about 140%, about 145%, or about 150% of the % U_{TM}. In this context, the term “uracil” can refer to an alternative uracil and/or naturally occurring uracil.

In some embodiments, the uracil content of the ORF of the polynucleotide relative to the uracil content of the corresponding wild-type ORF (% U_{WT}) is less than 100%. In some embodiments, the % U_{WT} of the polynucleotide is less than about 95%, less than about 90%, less than about 85%, less than 80%, less than 79%, less than 78%, less than 77%, less than 76%, less than 75%, less than 74%, or less than 73%. In some embodiments, the % U_{WT} of the polynucleotide is between 65% and 73%.

In some embodiments, the uracil content in the ORF of the mRNA encoding a is less than about 50%, about 40%, about 30%, or about 20% of the total nucleobase content in the ORF. In some embodiments, the uracil content in the ORF is between about 15% and about 25% of the total nucleobase content in the ORF. In other embodiments, the uracil content in the ORF is between about 20% and about 30% of the total nucleobase content in the ORF. In one embodiment, the uracil content in the ORF of the mRNA encoding a polypeptide is less than about 20% of the total nucleobase content in the open reading frame. In this context, the term “uracil” can refer to an alternative uracil and/or naturally occurring uracil.

In further embodiments, the ORF of the mRNA encoding a polypeptide having adjusted uracil content has increased cytosine (C), guanine (G), or guanine/cytosine (G/C) content (absolute or relative). In some embodiments, the overall increase in C, G, or G/C content (absolute or relative) of the ORF is at least about 2%, at least about 3%, at least about 4%, at least about 5%, at least about 6%, at least about 7%, at least about 10%, at least about 15%, at least about 20%, at least about 40%, at least about 50%, at least about 60%, at least about 70%, at least about 80%, at least about 90%, at least about 95%, or at least about 100% relative to the G/C content (absolute or relative) of the wild-type ORF. In some embodiments, the G, the C, or the G/C content in the ORF is less than about 100%, less than about 90%, less than about 85%, or less than about 80% of the theoretical maximum G, C, or G/C content of the nucleotide sequence encoding the PBDG polypeptide (% G_{TMX}; % C_{TMX}, or % G/C_{TMX}). In other embodiments, the G, the C, or the G/C content in the ORF is between about 70% and about 80%, between about 71% and about 79%, between about 71% and about 78%, or between about 71% and about 77% of the % G_{TMX}, % C_{TMX}, or % G/C_{TMX}. In some embodiments, the guanine content of the ORF of the polynucleotide with respect to the theoretical maximum guanine content of a nucleotide sequence encoding the polypeptide (% G_{TMX}) is at least 69%, at least 70%, at least 75%, at least about 80%, at least about 85%, at least about 90%, at least about 95%, or about 100%. In some embodiments, the % G_{TMX} of the polynucleotide is between about 70% and about 80%, between about 71% and about 79%, between about 71% and about 78%, or between about 71% and about 77%. In some embodiments, the cytosine content of the ORF of the polynucleotide relative to the theoretical maximum cytosine content of a nucleotide sequence encoding the polypeptide (% C_{TMX}) is at least 59%, at least 60%, at least about 65%, at least about 70%, at least about 75%, at least about 80%, at least about 85%, at least about 90%, at least about 95%, or about 100%. In some embodiments, the % C_{TMX} of the ORF of the polynucleotide is between about 60% and about 80%, between about 62% and about 80%, between about 63% and about 79%, or between about 68% and about 76%. In some embodiments, the guanine and cytosine content (G/C) of the ORF of the polynucleotide relative to the theoretical maximum G/C content in a nucleotide sequence encoding the polypeptide (% G/C_{TMX}) is at least about 81%, at least about 85%, at least about 90%, at least about 95%, or about 100%. In some embodiments, the % G/C_{TMX} in the ORF of the polynucleotide is between about 80% and about 100%, between about 85% and about 99%, between about 90% and about 97%, or between about 91% and about 96%. In some embodiments, the G/C content in the ORF of the polynucleotide relative to the G/C content in the corresponding wild-type ORF (% G/C_{WT}) is at least 102%, at least 103%, at least 104%, at least 105%, at least 106%, at least 107%, at least 110%, at least 115%, or at least 120%. In some embodiments, the average G/C content in the 3rd codon position in the ORF of the polynucleotide is at least 20%, at least 21%, at least 22%, at least 23%, at least 24%, at least 25%, at least 26%, at least 27%, at least 28%, at least 29%, or at least 30% higher than the average G/C content in the 3rd codon position in the corresponding wild-type ORF. In some embodiments, the increases in G and/or C content (absolute or relative) described herein can be conducted by replacing synonymous codons with low G, C, or G/C content with synonymous codons having higher G, C, or G/C content. In other embodiments, the increase in G and/or C content (absolute or relative) is conducted by replacing a codon ending with U with a synonymous codon ending with G or C.

In further embodiments, the ORF of the mRNA encoding a polypeptide includes less uracil pairs (UU) and/or uracil triplets (UUU) and/or uracil quadruplets (UUUU) than the corresponding wild-type nucleotide sequence encoding the polypeptide. In some embodiments, the ORF of the mRNA encoding a polypeptide of the disclosure includes no uracil pairs and/or uracil triplets and/or uracil quadruplets. In some embodiments, uracil pairs and/or uracil triplets and/or uracil quadruplets are reduced below a certain threshold, e.g., no more than 1, 2, 3, 4, 5, 6, 7, 8, 9, 10, 11, 12, 13, 14, 15, 16, 17, 18, 19, or 20 occurrences in the ORF of the mRNA encoding the polypeptide. In a particular embodiment, the ORF of the mRNA encoding the polypeptide of the disclosure contains less than 20, 19, 18, 17, 16, 15, 14, 13, 12, 11, 10, 9, 8, 7, 6, 5, 4, 3, 2, or 1 non-phenylalanine uracil pairs and/or triplets. In another embodiment, the ORF of the mRNA encoding the polypeptide contains no non-phenylalanine uracil pairs and/or triplets.

In further embodiments, the ORF of the mRNA encoding a polypeptide of the disclosure includes less uracil-rich clusters than the corresponding wild-type nucleotide sequence encoding the polypeptide. In some embodiments, the ORF of the mRNA encoding the polypeptide of the disclosure contains uracil-rich clusters that are shorter in length than corresponding uracil-rich clusters in the corresponding wild-type nucleotide sequence encoding the polypeptide.

In further embodiments, alternative lower frequency codons are employed. In some embodiment, the ORF of the polynucleotide further comprises at least one low-frequency codon. In some embodiments, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, or 100% of the codons in the polypeptide-encoding ORF of the mRNA are substituted with alternative codons, each alternative codon having a codon frequency lower than the codon frequency of the substituted codon in the synonymous codon set. The ORF may also have adjusted uracil content, as described above. In some embodiments, at least one codon in the ORF of the mRNA encoding the polypeptide is substituted with an alternative codon having a codon frequency lower than the codon frequency of the substituted codon in the synonymous codon set.

In some embodiments, the polynucleotide is an mRNA that comprises an ORF that encodes a polypeptide, wherein the uracil content of the ORF is between about 115% and about 135% of the theoretical minimum uracil content in the corresponding wild-type ORF, and wherein the uracil content in the ORF encoding the polypeptide is less than about 30% of the total nucleobase content in the ORF. In some embodiments, the ORF that encodes the polypeptide is further modified to increase G/C content of the ORF (absolute or relative) by at least about 40%, as compared to the corresponding wild-type ORF. In yet other embodiments, the ORF encoding the polypeptide contains less than 20 non-phenylalanine uracil pairs and/or triplets. In some embodiments, at least one codon in the ORF of the mRNA encoding the polypeptide is further substituted with an alternative codon having a codon frequency lower than the codon frequency of the substituted codon in the synonymous codon set.

In some embodiments, the expression of the polypeptide encoded by an mRNA comprising an ORF, wherein the uracil content of the ORF has been adjusted (e.g., the uracil content is between about 115% and about 135% of the theoretical minimum uracil content in the corresponding wild-type ORF) is increased by at least about 10-fold when compared to expression of the polypeptide from the corresponding wild-type mRNA. In some embodiments, the innate immune response induced by the mRNA including an open ORF wherein the uracil content has been adjusted (e.g., the uracil content of the ORF is between about 115% and about 135% of the theoretical minimum uracil content in the corresponding wild-type ORF) is reduced by at least about 10-fold when compared to expression of the polypeptide from the corresponding wild-type mRNA. In some embodiments, the mRNA with adjusted uracil content does not substantially induce an innate immune response of a mammalian cell into which the mRNA is introduced.

In some embodiments, the uracil content of the mRNA is adjusted as described herein, and a modified nucleoside is partially or completely substituted for the uracil remaining in the mRNA following adjustment. As a non-limiting example, the natural nucleotide uridine may be substituted with a modified nucleoside as described herein. In some embodiments, the modified nucleoside comprises pseudouridine (ψ). In some embodiments, the modified nucleoside comprises 1-methyl-pseudouridine (m1ψ). In some embodiments, the modified nucleoside comprises 1-methyl-pseudouridine (m1ψ) and 5-methyl-cytidine (m5C). In some embodiments, the modified nucleoside comprises 2-thiouridine (s2U). In some embodiments, the modified nucleoside comprises 2-thiouridine and 5-methyl-cytidine (m5C). In some embodiments, the modified nucleoside comprises 5-methoxy-uridine (mo5U). In some embodiments, the modified nucleoside comprises 5-methoxy-uridine (mo5U) and 5-methyl-cytidine (m5C). In some embodiments, the modified nucleoside comprises 2′-O-methyl uridine. In some embodiments, the modified nucleoside comprises 2′-O-methyl uridine and 5-methyl-cytidine (m5C). In some embodiments, the modified nucleoside comprises N6-methyl-adenosine (m6A). In some embodiments, the modified nucleoside comprises N6-methyl-adenosine (m6A) and 5-methyl-cytidine (m5C).

### Lipid Nanoparticle Formulations

In some embodiments, nucleic acids of the invention (e.g., mRNA encoding a secreted protein, e.g., mRNA encoding an intracellular protein) are formulated in a lipid nanoparticle (LNP). Lipid nanoparticles typically comprise ionizable cationic lipid, non-cationic lipid, sterol and PEG lipid components along with the nucleic acid cargo of interest. The lipid nanoparticles of the invention can be generated using components, compositions, and methods as are generally known in the art, see for example PCT/US2016/052352; PCT/US2016/068300; PCT/US2017/037551; PCT/US2015/027400; PCT/US2016/047406; PCT/US2016000129; PCT/US2016/014280; PCT/US2016/014280; PCT/US2017/038426; PCT/US2014/027077; PCT/US2014/055394; PCT/US2016/52117; PCT/US2012/069610; PCT/US2017/027492; PCT/US2016/059575 and PCT/US2016/069491 all of which are incorporated by reference herein in their entirety.

Nucleic acids of the present disclosure (e.g. TARGET mRNA) are typically formulated in lipid nanoparticle. In some embodiments, the lipid nanoparticle comprises at least one ionizable cationic lipid, at least one non-cationic lipid, at least one sterol, and/or at least one polyethylene glycol (PEG)-modified lipid.

In some embodiments, the lipid nanoparticle comprises a molar ratio of 20-60% ionizable cationic lipid. For example, the lipid nanoparticle may comprise a molar ratio of 20-50%, 20-40%, 20-30%, 30-60%, 30-50%, 30-40%, 40-60%, 40-50%, or 50-60% ionizable cationic lipid. In some embodiments, the lipid nanoparticle comprises a molar ratio of 20%, 30%, 40%, 50, or 60% ionizable cationic lipid.

In some embodiments, the lipid nanoparticle comprises a molar ratio of 5-25% non-cationic lipid. For example, the lipid nanoparticle may comprise a molar ratio of 5-20%, 5-15%, 5-10%, 10-25%, 10-20%, 10-25%, 15-25%, 15-20%, or 20-25% non-cationic lipid. In some embodiments, the lipid nanoparticle comprises a molar ratio of 5%, 10%, 15%, 20%, or25% non-cationic lipid.

In some embodiments, the lipid nanoparticle comprises a molar ratio of 25-55% sterol. For example, the lipid nanoparticle may comprise a molar ratio of 25-50%, 25-45%, 25-40%, 25-35%, 25-30%, 30-55%, 30-50%, 30-45%, 30-40%, 30-35%, 35-55%, 35-50%, 35-45%, 35-40%, 40-55%, 40-50%, 40-45%, 45-55%, 45-50%, or 50-55% sterol. In some embodiments, the lipid nanoparticle comprises a molar ratio of 25%, 30%, 35%, 40%, 45%, 50%, or 55% sterol.

In some embodiments, the lipid nanoparticle comprises a molar ratio of 0.5-15% PEG-modified lipid. For example, the lipid nanoparticle may comprise a molar ratio of 0.5-10%, 0.5-5%, 1-15%, 1-10%, 1-5%, 2-15%, 2-10%, 2-5%, 5-15%, 5-10%, or 10-15%. In some embodiments, the lipid nanoparticle comprises a molar ratio of 0.5%, 1%, 2%, 3%, 4%, 5%, 6%, 7%, 8%, 9%, 10%, 11%, 12%, 13%, 14%, or 15% PEG-modified lipid.

In some embodiments, the lipid nanoparticle comprises a molar ratio of 20-60% ionizable cationic lipid, 5-25% non-cationic lipid, 25-55% sterol, and 0.5-15% PEG-modified lipid.

In some embodiments, the lipid nanoparticle comprises a molar ratio of 45-49% Compound 1, 8-13% DSPC, 35-40% cholesterol, and 0.5-3.0 mol % Compound 2. In some embodiments, the lipid nanoparticle comprises a molar ratio of 48-49% Compound 1, 10-12% DSPC, 38-40% cholesterol, and 0.5-2.5 mol % Compound 2.

### Ionizable Lipids

In some aspects, the ionizable lipids of the present disclosure may be one or more of compounds of Formula (I):

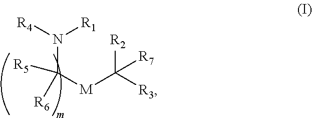

or their N-oxides, or salts or isomers thereof, wherein:

R₁ is selected from the group consisting of C₅₋₃₀ alkyl, C₅₋₂₀ alkenyl, —R*YR″, —YR″, and —R″M′R′;

R₂ and R₃ are independently selected from the group consisting of H, C₁₋₁₄ alkyl, C₂₋₁₄ alkenyl, —R*YR″, —YR″, and —R*OR″, or R₂ and R₃, together with the atom to which they are attached, form a heterocycle or carbocycle;

R₄ is selected from the group consisting of hydrogen, a C₃₋₆ carbocycle, —(CH₂)ₙQ, —(CH₂)ₙCHQR,

—CHQR, —CQ(R)₂, and unsubstituted C₁₋₆ alkyl, where Q is selected from a carbocycle, heterocycle, —OR, —O(CH₂)ₙN(R)₂, —C(O)OR, —OC(O)R, —CX₃, —CX₂H, —CXH₂, —CN, —N(R)₂, —C(O)N(R)₂, —N(R)C(O)R, —N(R)S(O)₂R, —N(R)C(O)N(R)₂, —N(R)C(S)N(R)₂, —N(R)R₈, —N(R)S(O)₂R₈, —O(CH₂)ₙOR, —N(R)C(═NR₉)N(R)₂, —N(R)C(═CHR₉)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)N(R)₂, —C(═NR₉)R, —C(O)N(R)OR, and —C(R)N(R)₂C(O)OR, and each n is independently selected from 1, 2, 3, 4, and 5;

each R₅ is independently selected from the group consisting of C₁₋₃ alkyl, C₂₋₃ alkenyl, and H;

each R₆ is independently selected from the group consisting of C₁₋₃ alkyl, C₂₋₃ alkenyl, and H;

M and M′ are independently selected from —C(O)O—, —OC(O)—, —OC(O)-M″-C(O)O—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group, in which M″ is a bond, C₁₋₁₃ alkyl or C₂₋₁₃ alkenyl; R₇ is selected from the group consisting of C₁₋₃ alkyl, C₂₋₃ alkenyl, and H; R₈ is selected from the group consisting of C₃₋₆ carbocycle and heterocycle; R₉ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;

each R is independently selected from the group consisting of C₁₋₃ alkyl, C₂₋₃ alkenyl, and

H;

each R′ is independently selected from the group consisting of C₁₋₁₈ alkyl, C₂₋₁₈ alkenyl, —R*YR″, —YR″, and H;

each R″ is independently selected from the group consisting of C₃₋₁₅ alkyl and C₃₋₁₅ alkenyl;

each R* is independently selected from the group consisting of C₁₋₁₂ alkyl and C₂₋₁₂ alkenyl;

each Y is independently a C₃₋₆ carbocycle;

each X is independently selected from the group consisting of F, Cl, Br, and I; and m is selected from 5, 6, 7, 8, 9, 10, 11, 12, and 13; and wherein when R₄ is (CH₂)ₙQ, (CH₂)ₙCHQR, —CHQR, or CQ(R)₂, then (i) Q is not N(R)₂ when n is 1, 2, 3, 4 or 5, or (ii) Q is not 5, 6, or 7-membered heterocycloalkyl when n is 1 or 2.

In one embodiment, the compounds of Formula (I) are of Formula (IIa),

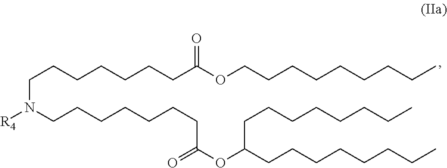

or their N-oxides, or salts or isomers thereof, wherein R₄ is as described herein.

In another embodiment, the compounds of Formula (I) are of Formula (IIb),

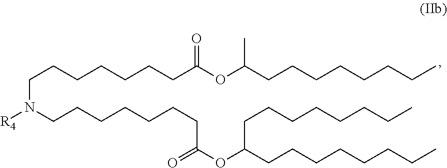

or their N-oxides, or salts or isomers thereof, wherein R₄ is as described herein.

In some embodiments, the ionizable lipid is Compound 1 shown below:

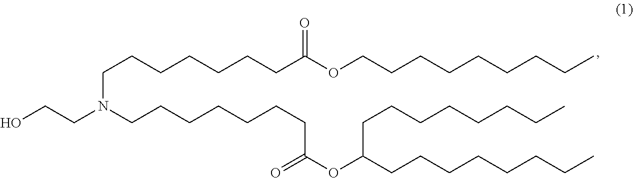 [SMILES: CCCCCCCCCOC(=O)CCCCCCCN(CCO)CCCCCCCC(=O)OC(CCCCCCCC)CCCCCCCC]

or a salt thereof.

### Phospholipids

The lipid composition of the lipid nanoparticle composition disclosed herein can comprise one or more phospholipids, for example, one or more saturated or (poly)unsaturated phospholipids or a combination thereof. In general, phospholipids comprise a phospholipid moiety and one or more fatty acid moieties.

A phospholipid moiety can be selected, for example, from the non-limiting group consisting of phosphatidyl choline, phosphatidyl ethanolamine, phosphatidyl glycerol, phosphatidyl serine, phosphatidic acid, 2-lysophosphatidyl choline, and a sphingomyelin.

A fatty acid moiety can be selected, for example, from the non-limiting group consisting of lauric acid, myristic acid, myristoleic acid, palmitic acid, palmitoleic acid, stearic acid, oleic acid, linoleic acid, alpha-linolenic acid, erucic acid, phytanoic acid, arachidic acid, arachidonic acid, eicosapentaenoic acid, behenic acid, docosapentaenoic acid, and docosahexaenoic acid.

Particular phospholipids can facilitate fusion to a membrane. For example, a cationic phospholipid can interact with one or more negatively charged phospholipids of a membrane (e.g., a cellular or intracellular membrane). Fusion of a phospholipid to a membrane can allow one or more elements (e.g., a therapeutic agent) of a lipid-containing composition (e.g., LNPs) to pass through the membrane permitting, e.g., delivery of the one or more elements to a target tissue.

Non-natural phospholipid species including natural species with modifications and substitutions including branching, oxidation, cyclization, and alkynes are also contemplated. For example, a phospholipid can be functionalized with or cross-linked to one or more alkynes (e.g., an alkenyl group in which one or more double bonds is replaced with a triple bond). Under appropriate reaction conditions, an alkyne group can undergo a copper-catalyzed cycloaddition upon exposure to an azide. Such reactions can be useful in functionalizing a lipid bilayer of a nanoparticle composition to facilitate membrane permeation or cellular recognition or in conjugating a nanoparticle composition to a useful component such as a targeting or imaging moiety (e.g., a dye).

Phospholipids include, but are not limited to, glycerophospholipids such as phosphatidylcholines, phosphatidylethanolamines, phosphatidylserines, phosphatidylinositols, phosphatidy glycerols, and phosphatidic acids. Phospholipids also include phosphosphingolipid, such as sphingomyelin.

In some embodiments, a phospholipid of the invention comprises 1,2-distearoyl-sn-glycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-gly cero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2 cholesterylhemisuccinoyl-sn-glycero-3-phosphocholine (OChemsPC), 1-hexadecyl-sn-glycero-3-phosphocholine (C16 Ly so PC), 1,2-dilinolenoyl-sn-glycero-3-phosphocholine,1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin, and mixtures thereof.

### Structural Lipids

The lipid composition of a pharmaceutical composition disclosed herein can comprise one or more structural lipids. As used herein, the term “structural lipid” refers to sterols and also to lipids containing sterol moieties.

Incorporation of structural lipids in the lipid nanoparticle may help mitigate aggregation of other lipids in the particle. Structural lipids can be selected from the group including but not limited to, cholesterol, fecosterol, sitosterol, ergosterol, campesterol, stigmasterol, brassicasterol, tomatidine, tomatine, ursolic acid, alpha-tocopherol, hopanoids, phytosterols, steroids, and mixtures thereof. In some embodiments, the structural lipid is a sterol. As defined herein, “sterols” are a subgroup of steroids consisting of steroid alcohols. In certain embodiments, the structural lipid is a steroid. In certain embodiments, the structural lipid is cholesterol. In certain embodiments, the structural lipid is an analog of cholesterol. In certain embodiments, the structural lipid is alpha-tocopherol.

### Polyethylene Glycol (PEG)-Lipids

The lipid composition of a pharmaceutical composition disclosed herein can comprise one or more a polyethylene glycol (PEG) lipid.

As used herein, the term “PEG-lipid” refers to polyethylene glycol (PEG)-modified lipids. Non-limiting examples of PEG-lipids include PEG-modified phosphatidylethanolamine and phosphatidic acid, PEG-ceramide conjugates (e.g., PEG-CerC14 or PEG-CerC20), PEG-modified dialkylamines and PEG-modified 1,2-diacyloxypropan-3-amines. Such lipids are also referred to as PEGylated lipids. For example, a PEG lipid can be PEG-c-DOMG, PEG-DMG, PEG-DLPE, PEGDMPE, PEG-DPPC, or a PEG-DSPE lipid.

In some embodiments, the PEG-lipid includes, but not limited to 1,2-dimyristoyl-sn-glycerol methoxypolyethylene glycol (PEG-DMG), 1,2-distearoyl-sn-glycero-3-phosphoethanolamine-N-[amino(polyethylene glycol)] (PEG-DSPE), PEG-disteryl glycerol (PEG-DSG), PEG-dipalmetoleyl, PEG-dioleyl, PEG-distearyl, PEG-diacylglycamide (PEG-DAG), PEG-dipalmitoyl phosphatidylethanolamine (PEG-DPPE), or PEG-1,2-dimyristyloxlpropyl-3-amine (PEG-c-DMA).

In certain embodiments, a PEG lipid useful in the present invention is a PEGylated fatty acid. In certain embodiments, a PEG lipid useful in the present invention is a compound of Formula (III).

Provided herein are compounds of Formula (III):

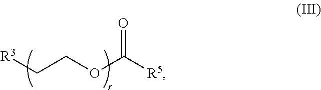

or a salts thereof, wherein:
R³ is —OR^{O};
R^{O} is hydrogen, optionally substituted alkyl or an oxygen protecting group;
r is an integer between 1 and 100, inclusive;
R⁵ is optionally substituted C₁₀₋₄₀ alkyl, optionally substituted C₁₀₋₄₀ alkenyl, or optionally substituted C₁₀₋₄₀ alkynyl; and optionally one or more methylene groups of R⁵ are replaced with optionally substituted carbocyclylene, optionally substituted heterocyclylene, optionally substituted arylene, optionally substituted heteroarylene, —N(R^{N}), O, S, C(O), C(O)N(R^{N}), NR^{N}C(O), NR^{N}C(O)N(R^{N}), C(O)O, OC(O), OC(O)O, OC(O)N(R^{N}), NR^{N}C(O)O, C(O)S, SC(O), C(═NR^{N}), C(═NR^{N})N(R^{N}), NR^{N}C(═NR^{N}), —NR^{N}C(═NR^{N})N(R^{N}), C(S), C(S)N(R^{N}), NR^{N}C(S), NR^{N}C(S)N(R^{N}), S(O), OS(O), S(O)O, OS(O)O, OS(O)₂, S(O)₂O, OS(O)₂O, N(R^{N})S(O), S(O)N(R^{N}), N(R^{N})S(O)N(R^{N}), —OS(O)N(R^{N}), N(R^{N})S(O)O, S(O)₂, N(R^{N})S(O)₂, S(O)₂N(R^{N}), N(R^{N})S(O)₂N(R^{N}), —OS(O)₂N(R^{N}), or N(R^{N})S(O)₂O; and
each instance of R^{N} is independently hydrogen, optionally substituted alkyl, or a nitrogen protecting group.

In certain embodiments, the compound of Formula (III) is of Formula (III-OH):

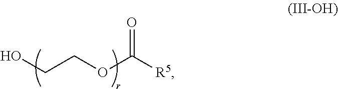

or a salt thereof. In some embodiments, r is 45.

In yet other embodiments the compound of Formula (III) is:

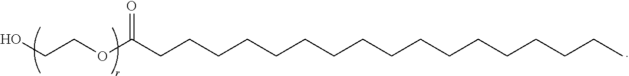

or a salt thereof.

In one embodiment, the compound of Formula (III) is Compound 2 shown below:

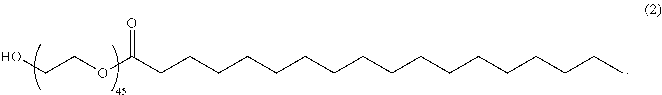

In some embodiments, a LNP of the invention comprises an ionizable cationic lipid of

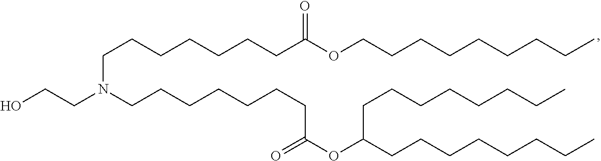 [SMILES: CCCCCCCCCOC(=O)CCCCCCCN(CCO)CCCCCCCC(=O)OC(CCCCCCCC)CCCCCCCC]

and a PEG lipid comprising Formula III.

In some embodiments, a LNP of the invention comprises an ionizable cationic lipid of Compound 1 and an PEG lipid of Compound 2.

In some embodiments, a LNP of the invention comprises an N:P ratio of from about 2:1 to about 30:1.

In some embodiments, a LNP of the invention comprises an N:P ratio of about 6:1.

In some embodiments, a LNP of the invention comprises an N:P ratio of about 3:1.

In some embodiments, a LNP of the invention comprises a wt/wt ratio of the ionizable cationic lipid component to the RNA of from about 10:1 to about 100:1.

In some embodiments, a LNP of the invention comprises a wt/wt ratio of the ionizable cationic lipid component to the RNA of about 20:1.

In some embodiments, a LNP of the invention comprises a wt/wt ratio of the ionizable cationic lipid component to the RNA of about 10:1.

In some embodiments, a LNP of the invention has a mean diameter from about 50 nm to about 150 nm.

In some embodiments, a LNP of the invention has a mean diameter from about 70 nm to about 120 nm.

Methods of Determining mRNA Therapeutic Effect

### Measuring Expression of an Encoded Polypeptide

In some aspects, the disclosure provides mRNAs comprising an ORF encoding a polypeptide of interest. Methods for determining polypeptide expression and/or activity are known to those of skill in the art and are described herein. Such methods include, but are not limited to, quantitative immunofluorescence (QIF), flow cytometry, reverse transcription polymerase chain reaction (RT-PCR), competitive RT-PCR, real-time RT-PCR, RNase protection assay (RPA), northern blotting, nucleic acid microarray using DNA, western blotting, enzyme-linked immunosorbent assay (ELISA), radioimmunoassay (RIA), tissue immunostaining, immunoprecipitation assay, complement fixation assay, fluorescence-activated cell sorting (FACS), mass spectrometry, magnetic bead-antibody immunoprecipitation, protein chip, or biochemical or biomarker assays to determine enzymatic activity in vitro or in vivo.

Certain aspects of the disclosure feature measurement, determination and/or monitoring of the expression level or levels of an encoded polypeptide of interest in a subject, for example, in an animal (e.g., rodents, primates, and the like) or in a human subject. Animals include normal, healthy or wild-type animals, as well as animal models for use in understanding the pathophysiology or disease state resulting from the deficiency of a polypeptide of interest (e.g., a therapeutic protein, such as an intracellular or secreted protein).

Expression levels of an encoded polypeptide of interest can be measured or determined by any art-recognized method for determining protein levels in biological samples, e.g., from blood samples or a needle biopsy. It will be understood by the skilled artisan that in certain embodiments the sample may be subjected, e.g., to any of the following: purification, precipitation, separation, e.g. centrifugation and/or HPLC, and subsequently subjected to determining the level of the protein, e.g., using mass and/or spectrometric analysis. In exemplary embodiments, enzyme-linked immunosorbent assay (ELISA) can be used to determine protein expression levels. In other exemplary embodiments, protein purification, separation and LC-MS can be used as a means for determining the level of a protein according to the invention. In some embodiments, the level of expression of a polypeptide of interest is determined in any tissue collected from a subject, non-limiting examples including bone, blood, heart, kidney, liver, skin, intestine, brain, spleen, thyroid, or lung.

In some embodiments, an mRNA therapy of the disclosure (e.g., a single intravenous dose) results in increased expression level of a polypeptide of interest in a given tissue of the subject (e.g., liver, kidney, or heart) that is 2-fold, 3-fold, 4-fold, 5-fold, 6-fold, 7-fold, 8-fold, 9-fold, 10-fold, 20-fold, 30-fold, 40-fold, 50-fold increase and/or increased to at least 50%, at least 60%, at least 70%, at least 75%, 80%, at least 85%, at least 90%, at least 95%, or at least 100% of normal levels for at least 6 hours, at least 12 hours, at least 24 hours, at least 36 hours, at least 48 hours, at least 60 hours, at least 72 hours, at least 84 hours, at least 96 hours, at least 108 hours, at least 122 hours, at least 144 hours, at least 168 hours, at least 192 hours, at least 240 hours, at least 288 hours, at least 336 hours, at least 384 hours, at least 432 hours, at least 480 hours, at least 504 hours at least 528 hours, at least 672 hours after administration of a single dose of the mRNA therapy. In some embodiments, an mRNA therapy of the disclosure (e.g., a single intravenous dose) results in increased expression level of a polypeptide of interest in a given tissue of the subject (e.g., liver, kidney, or heart) that is 2-fold, 3-fold, 4-fold, 5-fold, 6-fold, 7-fold, 8-fold, 9-fold, 10-fold, 20-fold, 30-fold, 40-fold, 50-fold increase and/or increased to at least 50%, at least 60%, at least 70%, at least 75%, 80%, at least 85%, at least 90%, at least 95%, or at least 100% of normal levels for at least 6 hours, at least 12 hours, at least 24 hours, or at least 2, 3, 4, 5, 6, 7, 8, 9, 10, 11, 12, 13, 14, 15, 16, 17, 18, 19, 20, 21, 22, 23, 24, 25, 26, 27, 28, 29, or 30 or more days after administration of a single dose of the mRNA therapy.

### Measuring Activity of a Translated Protein

In some patients with a disease or disorder, the activity of a polypeptide of interest is reduced compared to a normal physiological activity level. Further aspects of the disclosure feature measurement, determination and/or monitoring of the activity level(s) of a polypeptide of interest in a subject, for example, in an animal (e.g., rodent, primate, and the like) or in a human subject.

Activity levels can be measured or determined by any art-recognized method for determining activity levels (e.g., enzymatic activity levels) in biological samples. In certain embodiments, an mRNA therapy of the disclosure features a pharmaceutical composition comprising a dose of mRNA effective to result in at least 5 U/mg, at least 10 U/mg, at least 20 U/mg, at least 30 U/mg, at least 40 U/mg, at least 50 U/mg, at least 60 U/mg, at least 70 U/mg, at least 80 U/mg, at least 90 U/mg, at least 100 U/mg, or at least 150 U/mg of activity in tissue (e.g., liver) between 6 and 12 hours, or between 12 and 24, between 24 and 48, or between 48 and 72 hours post administration (e.g., at 48 or at 72 hours post administration).

In some embodiments, an mRNA therapy of the disclosure (e.g., a single intravenous dose) results in increased activity levels in the liver, kidney or heart tissue of the subject (e.g., 2-fold, 3-fold, 4-fold, 5-fold, 6-fold, 7-fold, 8-fold, 9-fold, 10-fold, 20-fold, 30-fold, 40-fold, 50-fold increase and/or increased to at least 50%, at least 60%, at least 70%, at least 75%, 80%, at least 85%, at least 90%, at least 95%, or at least 100% of normal levels) for at least 6 hours, at least 12 hours, at least 24 hours, or at least 2, 3, 4, 5, 6, 7, 8, 9, 10, 11, 12, 13, 14, 15, 16, 17, 18, 19, 20, 21, 22, 23, 24, 25, 26, 27, 28, 29, or 30 or more days after administration of a single dose of the mRNA therapy.

In exemplary embodiments, an mRNA therapy of the disclosure features a pharmaceutical composition comprising a single intravenous dose of mRNA that results in the above-described levels of activity. In another embodiment, an mRNA therapy of the disclosure features a pharmaceutical composition which can be administered in multiple single unit intravenous doses of mRNA that maintain the above-described levels of activity.

### Measuring Biomarkers of a Translated Polypeptide of Interest

Further aspects of the disclosure feature determining the level (or levels) of a biomarker, (e.g., a metabolite or enzymatic product produced by a cellular enzyme) determined in a sample as compared to a level (e.g., a reference level) of the same or another biomarker in another sample, e.g., from the same patient, from another patient, from a control and/or from the same or different time points, and/or a physiologic level, and/or an elevated level, and/or a supraphysiologic level, and/or a level of a control. The skilled artisan will be familiar with physiologic levels of biomarkers, for example, levels in normal or wild-type animals, normal or healthy subjects, and the like, in particular, the level or levels characteristic of subjects who are healthy and/or normal functioning.

In one embodiment, a control is a sample of a healthy patient. In another embodiment, the control is a sample from at least one subject having a known disease status, for example, a severe, mild, or healthy disease status, e.g. a control patient. In another embodiment, the control is a sample from a subject not being treated for the disease. In a still further embodiment, the control is a sample from a single subject or a pool of samples from different subjects and/or samples taken from the subject(s) at different time points.

Biomarkers of the disclosure include, for example, metabolites or enzymatic products of a cellular enzyme. It will be understood by the skilled artisan that in certain embodiments the sample may be subjected to, e.g., one or more of the following: substance purification, precipitation, separation, e.g. centrifugation and/or HPLC and subsequently subjected to determining the level of the biomarker, e.g. using mass spectrometric analysis. In certain embodiments, LC-MS can be used as a means for determining the level of a biomarker according to the disclosure.

In some embodiments, comparing the level of the biomarker in a sample from a subject in need of treatment for a disease or disorder (e.g., a disease or disorder resulting from an enzyme deficiency) or in a subject being treated for a disease or disorder (e.g., a disease or disorder resulting from an enzyme deficiency) to a control level of the biomarker comprises comparing the level of the biomarker in the sample from the subject (e.g., in need of treatment or being treated for the disease or disorder) to a baseline or reference level, wherein if a level of the biomarker in the sample from the subject (e.g., in need of treatment or being treated for a disease or disorder) is elevated, increased or higher compared to the baseline or reference level, this is indicative that the subject is suffering from the disease or disorder and/or is in need of treatment; and/or wherein if a level of the biomarker in the sample from the subject (e.g., in need of treatment or being treated for the disease or disorder) is decreased or lower compared to the baseline level this is indicative that the subject is not suffering from, is successfully being treated for the disease or disorder (e.g., a disease or disorder resulting from an enzyme deficiency), or is not in need of treatment for the disease or disorder (e.g., a disease or disorder resulting from an enzyme deficiency). The stronger the reduction (e.g., at least 1.5-fold, at least 2-fold, at least 3-fold, at least 4-fold, at least 5-fold, at least 6-fold, at least 7-fold, at least 8-fold, at least 10-fold, at least 20-fold, at least-30 fold, at least 40-fold, at least 50-fold reduction and/or at least 10%, at least 20%, at least 30% at least 40%, at least 50%, at least 60%, at least 70%, at least 80%, at least 90%, or at least 100% reduction) of the level of a biomarker, within a certain time period, e.g., within 6 hours, within 12 hours, 24 hours, 36 hours, 48 hours, 60 hours, or 72 hours, and/or for a certain duration of time, e.g., 48 hours, 72 hours, 96 hours, 120 hours, 144 hours, 1 week, 2 weeks, 3 weeks, 4 weeks, 1 month, 2 months, 3 months, 4 months, 5 months, 6 months, 7 months, 8 months, 9 months, 10 months, 11 months, 12 months, 18 months, 24 months, etc. the more successful is a therapy, such as for example an mRNA therapy of the disclosure (e.g., a single dose or a multiple regimen).

A reduction of at least about 20%, at least about 30%, at least about 40%, at least about 50%, at least about 60%, at least about 70%, at least about 80%, at least about 90%, at least 100% or more of the level of biomarker, in particular, in bodily fluid (e.g., plasma, serum, red blood cells (RBC), urine, e.g., urinary sediment) or in tissue(s) in a subject (e.g., liver) within 1, 2, 3, 4, 5, 6 or more days following administration is indicative of a dose suitable for successful treatment the disease or disorder, wherein reduction as used herein, preferably means that the level of biomarker determined at the end of a specified time period (e.g., post-administration, for example, of a single intravenous dose) is compared to the level of the same biomarker determined at the beginning of said time period (e.g., pre-administration of said dose). Exemplary time periods include 12, 24, 48, 72, 96, 120 or 144 hours post administration, in particular 24, 48, 72 or 96 hours post administration.

### Pharmaceutical Compositions

The present disclosure includes pharmaceutical compositions comprising an mRNA or a nanoparticle (e.g., a lipid nanoparticle) described herein, in combination with one or more pharmaceutically acceptable excipient, carrier or diluent. In particular embodiments, the mRNA is present in a nanoparticle, e.g., a lipid nanoparticle. In particular embodiments, the mRNA or nanoparticle is present in a pharmaceutical composition.

Pharmaceutical compositions may optionally include one or more additional active substances, for example, therapeutically and/or prophylactically active substances. Pharmaceutical compositions of the present disclosure may be sterile and/or pyrogen-free. General considerations in the formulation and/or manufacture of pharmaceutical agents may be found, for example, in Remington: The Science and Practice of Pharmacy 21st ed., Lippincott Williams & Wilkins, 2005 (incorporated herein by reference in its entirety). In particular embodiments, a pharmaceutical composition comprises an mRNA and a lipid nanoparticle, or complexes thereof.

The mRNAs of the disclosure can be formulated using one or more excipients to: (1) increase stability; (2) increase cell transfection; (3) permit the sustained or delayed release (e.g., from a depot formulation of the mRNA); (4) alter the biodistribution (e.g., target the mRNA to specific tissues or cell types); (5) increase the translation of a polypeptide encoded by the mRNA in vivo; and/or (6) alter the release profile of a polypeptide encoded by the mRNA in vivo. In addition to traditional excipients such as any and all solvents, dispersion media, diluents, or other liquid vehicles, dispersion or suspension aids, surface active agents, isotonic agents, thickening or emulsifying agents, preservatives, excipients of the present disclosure can include, without limitation, lipidoids, liposomes, lipid nanoparticles (e.g., liposomes and micelles), polymers, lipoplexes, core-shell nanoparticles, peptides, proteins, carbohydrates, cells transfected with mRNAs (e.g., for transplantation into a subject), hyaluronidase, nanoparticle mimics and combinations thereof. Accordingly, the formulations of the disclosure can include one or more excipients, each in an amount that together increases the stability of the mRNA, increases cell transfection by the mRNA, increases the expression of a polypeptide encoded by the mRNA, and/or alters the release profile of an mRNA-encoded polypeptide. Further, the mRNAs of the present disclosure may be formulated using self-assembled nucleic acid nanoparticles.

In some embodiments, the formulations described herein may contain at least one type of mRNA. As a non-limiting example, the formulations may contain 1, 2, 3, 4, 5 or more than 5 mRNAs described herein. In some embodiments, the formulations described herein may contain at least one mRNA encoding a polypeptide and at least one nucleic acid sequence such as, but not limited to, an siRNA, an shRNA, a snoRNA, and an miRNA.

### Kits

In some embodiments, the disclosure provides a kit comprising an mRNA, or composition (e.g. lipid nanoparticle) comprising an mRNA as described herein. In some embodiments, a kit comprises a container comprising a pharmaceutical composition comprising a lipid nanoparticle comprising an mRNA described herein; and a pharmaceutically acceptable carrier, and a package insert comprising instructions for administration of the mRNA.

In some embodiments, a kit comprises a container comprising a pharmaceutical composition comprising a lipid nanoparticle comprising an mRNA encoding a polypeptide described herein; and a pharmaceutically acceptable carrier, and a package insert comprising instructions for administration of the mRNA and instruction for use in combination with a second composition comprising a second therapeutic agent.

In some embodiments, the disclosure provides a kit comprising a container comprising an LNP-formulated mRNA, an optional pharmaceutically acceptable carrier, and a package insert comprising instructions for producing a therapeutic level of an antibody in a human subject, wherein the mRNA encodes the antibody.

In some embodiments, the disclosure provides a kit comprising a container comprising an LNP-formulated mRNA, an optional pharmaceutically acceptable carrier, and a package insert comprising instructions for administration of the LNP-formulated mRNA to a subject for preventing a nosomcomial infection resulting from an infectious agent, wherein the mRNA encodes an antibody targeting the infectious agent. In some embodiments, the instructions further comprise administering the LNP-formulated mRNA prior to the subject being admitted to a hospital for an inpatient procedure (e.g., a surgical procedure).

In some embodiments, the disclosure provides a kit comprising a container comprising an LNP-formulated mRNA, an optional pharmaceutically acceptable carrier, and a package insert comprising instructions for administration of the LNP-formulated mRNA to a subject for preventing the onset or development of a seasonal respiratory virus in the subject, wherein the mRNA encodes an antibody targeting the virus. In some embodiments, the instructions further comprise administering the LNP-formulated mRNA prior to or concurrent with a seasonal epidemic caused by the virus.

In some embodiments, the disclosure provides a kit comprising a container comprising an LNP-formulated mRNA, an optional pharmaceutically acceptable carrier, and a package insert comprising instructions for administration of the LNP-formulated mRNA to a subject for preventing an endemic infectious disease, wherein the mRNA encodes an antibody targeting the infectious disease. In some embodiments, the instructions further comprise administering the LNP-formulated mRNA prior to the subject traveling to a region with a high prevalence of the disease.

In some embodiments, the disclosure provides a kit comprising a container comprising an LNP-formulated mRNA, an optional pharmaceutically acceptable carrier, and a package insert comprising instructions for administration of the LNP-formulated mRNA to a subject for preventing a cytokine storm, wherein the mRNA encodes an antibody targeting an inflammatory cytokine.

### Definitions

Unless otherwise defined herein, scientific and technical terms used in connection with the present disclosure shall have the meanings that are commonly understood by those of ordinary skill in the art. Further, unless otherwise required by context, singular terms shall include pluralities and plural terms shall include the singular.

Activity: As used herein, the term “activity” refers to an activity of a polypeptide (e.g., an enzyme) encoded by an mRNA of the disclosure. As is generally known by one skilled in the art, enzymes are macromolecular biological catalysts that accelerate biochemical reactions. The molecule upon which a enzyme acts is called a substrate and the enzyme converts the substrates into different molecules known as products. In some embodiments, the activity of a polypeptide is the conversion of a substrate into a product. The activity of a polypeptide is determined by any suitable method known in the art. In some embodiments, the activity of a polypeptide is determined by measuring the enzymatic activity or specific enzymatic activity of the polypeptide. In some embodiments, the activity of a polypeptide is determined by detecting the presence or determining an amount of product formed by the polypeptide in a sample or a subject.

Enzymatic activity is a measure of a quantity of active enzyme and is expressed in enzyme units (“U”) per volume, mass or weight of a sample or total protein within a sample. 1 enzyme unit (U) is defined as the amount of enzyme that catalyzes the conversion of one nanomole of substrate per hour (nmol/hr) under the specified conditions of an enzyme assay. The specified conditions are typically the optimum conditions that yield the maximal substrate conversion rate for a particular enzyme, and may include, but not be limited to, optimal temperature, pH and substrate concentration. In exemplary embodiments, the activity of a polypeptide (e.g., an enzyme) is described in terms of units (U) per milliliter (mL) of fluid (e.g., bodily fluid, e.g., serum, plasma, urine and the like) or is described in terms of units (U) per weight of tissue or per weight of protein (e.g., total protein) within a sample. In some embodiments, the activity of a polypeptide encoded by an mRNA of the disclosure is described in terms of U/mL plasma or U/mg protein (tissue). In some embodiments, the activity of a polypeptide encoded by an mRNA of the disclosure is described in terms of U/mL cell lysate or U/mL of tissue homogenate.

Some aspects of the disclosure feature measurement, determination and/or monitoring of the activity of a polypeptide encoded by an mRNA, such as those described herein, in a sample or a subject, for example, in animals (e.g., rodents, primates, and the like) or in human subjects. In some embodiments, an activity of a polypeptide encoded by an mRNA of the disclosure is measured, determined, or monitored by any art-recognized method for determining enzymatic activity in biological samples. In some embodiments, an activity of a polypeptide encoded by an mRNA of the disclosure is measured, determined, or monitored by any art-recognized method for detecting the presence or determining an amount of product formed by the polypeptide in biological samples. The skilled artisan will appreciate that the mRNAs provided by the disclosure can be characterized or determined by measuring the activity of a polypeptide (e.g., enzyme) encoded by the mRNA in a sample or in samples taken from a subject (e.g., from a preclinical test subject (rodent, primate, etc.) or from a clinical subject (human).

Administering: As used herein, “administering” refers to a method of delivering a composition to a subject or patient. A method of administration may be selected to target delivery (e.g., to specifically deliver) to a specific region or system of a body. For example, an administration may be parenteral (e.g., subcutaneous, intracutaneous, intravenous, intraperitoneal, intramuscular, intraarticular, intraarterial, intrasynovial, intrasternal, intrathecal, intralesional, or intracranial injection, as well as any suitable infusion technique), oral, trans- or intra-dermal, interdermal, rectal, intravaginal, topical (e.g., by powders, ointments, creams, gels, lotions, and/or drops), mucosal, nasal, buccal, enteral, vitreal, intratumoral, sublingual, intranasal; by intratracheal instillation, bronchial instillation, and/or inhalation; as an oral spray and/or powder, nasal spray, and/or aerosol, and/or through a portal vein catheter.

Approximately, about: As used herein, the terms “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Biomarker As used herein, the term “biomarker” (alternatively “response biomarker”) refers to a substance that is detected and/or measured in a sample or subject as an indicator of an activity of a polypeptide. For example, in some aspects, a biomarker is a product formed by the activity of a polypeptide encoded by an mRNA provided by the disclosure. In other aspects, a biomarker is a receptor whose expression and/or activity changes in response to the activity of a polypeptide encoded by an mRNA of the disclosure. In some aspects, the activity of a polypeptide is characterized or determined by measuring the level of an appropriate biomarker in sample(s) taken from a subject. The term “level of a biomarker” as used herein, preferably means the mass, weight or concentration of a biomarker, for example, a product formed by the activity of a polypeptide encoded by an mRNA, described herein, within a sample or a subject. It will be understood by the skilled artisan that in certain embodiments the sample may be subjected, e.g. to a step of substance purification, precipitation, separation, e.g. centrifugation and/or HPLC and subsequently subjected to a step of determining the level of the biomarker, e.g. using mass spectrometric analysis. In exemplary embodiments, LC-MS can be used as a means for determining the level of a biomarker according to the invention.

Coefficient of Variation (CV): As used herein, the term “coefficient of variation” refers to a statistical measure of the dispersion of data points in a data series relative to the average of the data points. As known to the skilled artisan, the coefficient of variation represents the ratio of the standard deviation and average obtained from a set of data points in a data series and provides a measure of the relative variability in the date set. For example, for a given data set A and a given data set B, the data set with a higher CV value represents the data set with a greater degree of variability. In some embodiments, following administration of an mRNA encoding a therapeutic polypeptide in more than one subject, a CV is used as a measure of variability between subjects for the therapeutic level of the encoded therapeutic polypeptide measured in serum or tissue. In some embodiments, when used to assess the therapeutic level of a therapeutic polypeptide in multiple subjects, a CV value below about 30%, 25%, 20%, 15%, 10%, or 5% is indicative of low variability.

Comparing: As used herein, the term “comparing” or “compared to” preferably means the mathematical comparison of the two or more values, e.g., of the levels of the biomarker(s). It will thus be readily apparent to the skilled artisan whether one of the values is higher, lower or identical to another value or group of values if at least two of such values are compared with each other. Comparing or comparison to can be in the context, for example, of comparing to a control value, e.g., as compared to a reference blood plasma, serum, red blood cells (RBC) and/or tissue (e.g., liver, kidney, heart) biomarker level, and/or a reference serum, blood plasma, tissue (e.g., liver, kidney, or heart), and/or urinary biomarker level, in said subject prior to administration (e.g., in a person suffering from a disease or disorder resulting from an enzyme deficiency) or in a normal or healthy subject. Comparing or comparison to can also be in the context, for example, of comparing to a control value, e.g., as compared to a reference blood plasma, serum, red blood cells (RBC) and/or tissue (e.g., liver, kidney, or heart) biomarker level, and/or a reference serum, blood plasma, tissue (e.g., liver), and/or urinary biomarker level in said subject prior to administration (e.g., in a person suffering from a disease or disorder resulting from an enzyme deficiency) or in a normal or healthy subject.

Determining the level: As used herein , the term “determining the level” of a substance (e.g., biomarker) refers to methods to quantify an amount of the substance in a sample, for example, from a subject (e.g., a bodily fluid; e.g., blood, lymph, serum, plasma, urine, etc.) or in a tissue of the subject (e.g., liver, heart, spleen kidney, etc.).

Encapsulate: As used herein, the term “encapsulate” means to enclose, surround, or encase. In some embodiments, a compound, an mRNA, or other composition may be fully encapsulated, partially encapsulated, or substantially encapsulated. For example, in some embodiments, an mRNA of the disclosure may be encapsulated in a lipid nanoparticle, e.g., a liposome.

Effective amount: As used herein, the term “effective amount” of an agent is that amount sufficient to effect beneficial or desired results, for example, clinical results, and, as such, an “effective amount” depends upon the context in which it is being applied. For example, in the context of administering an agent that treats cancer, an effective amount of an agent is, for example, an amount sufficient to achieve treatment, as defined herein, of cancer, as compared to the response obtained without administration of the agent. In some embodiments, a therapeutically effective amount is an amount of an agent to be delivered (e.g., nucleic acid, drug, therapeutic agent, diagnostic agent or prophylactic agent) that is sufficient, when administered to a subject suffering from or susceptible to an infection, disease, disorder, and/or condition, to treat, improve symptoms of, diagnose, prevent, and/or delay the onset of the infection, disease, disorder, and/or condition.

Frequency: As used herein, the term “frequency” refers to the rate of repeated administration of a drug (e.g, an LNP-formulated mRNA) over a certain period of time. For example, in some embodiments, administration of a dose of drug (e.g., an LNP-formulated mRNA) is repeated at a frequency that is daily, weekly, bi-weekly, monthly, etc. over a defined period of time or indefinitely.

Modified. As used herein “modified” refers to a changed state or structure of a molecule of the disclosure. Molecules may be modified in many ways including chemically, structurally, and functionally. In one embodiment, the mRNA molecules of the present disclosure are modified by the introduction of non-natural nucleosides and/or nucleotides, e.g., as it relates to the natural ribonucleotides A, U, G, and C. Noncanonical nucleotides such as the cap structures are not considered “modified” although they differ from the chemical structure of the A, C, G, U ribonucleotides.

Normal subject: As used herein, the term “normal subject” or “healthy subject” refers to a subject not suffering from symptoms associated with a disease or disorder (e.g., a disease or disorder resulting from an enzyme deficiency). Moreover, a subject will be considered to be normal (or healthy) if it has no mutation of the functional portions or domains of a polypeptide of interest and/or no mutation of the polypeptide of interest gene resulting in a reduction of or deficiency of the polypeptide of interest expression level or the activity thereof. Said mutations will be detected if a sample from the subject is subjected to a genetic testing for such mutations. In certain embodiments of the present disclosure, a sample from a healthy subject is used as a control sample, or the known or standardized value for the level of biomarker from samples of healthy or normal subjects is used as a control.

Patient: As used herein, “patient” refers to a subject who may seek or be in need of treatment, requires treatment, is receiving treatment, will receive treatment, or a subject who is under care by a trained professional for a particular disease or condition. In particular embodiments, a patient is a human patient. In some embodiments, a patient is a patient suffering from cancer (e.g., liver cancer or colorectal cancer).

Pharmaceutically acceptable: The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio

Reference level: The term “reference level” as used herein can refer to levels (e.g., of a biomarker) in a subject prior to administration of an mRNA therapy of the disclosure (e.g., in a person suffering from a disease or disorder resulting from an enzyme deficiency) or in a normal or healthy subject.

Subject: As used herein, the term “subject” refers to any organism to which a composition in accordance with the disclosure may be administered, e.g., for experimental, diagnostic, prophylactic, and/or therapeutic purposes. Typical subjects include animals (e.g., mammals such as mice, rats, rabbits, non-human primates, and humans) and/or plants. In some embodiments, a subject may be a human patient having ovarian cancer.

Systemic administration: As used herein, the term “systemic administration” refers to any route of administration (e.g., intravenous, subcutaneous, intracutaneous, intravenous, intraperitoneal, intramuscular, etc.) of a drug (e.g, an LNP-formulated mRNA) that results in the drug entering the circulatory system and distributing throughout the body.

Therapeutic ceiling: As used herein, the term “therapeutic ceiling” refers to the concentration of expressed therapeutic protein at which the maximum tolerated side effects occur.

Therapeutic level: As used herein, the term “therapeutic expression level”, or “therapeutic level” or “expression level” preferably means an amount (weight or mass) or concentration of a therapeutic polypeptide translated from an RNA (e.g., an mRNA) within the serum or a given tissue sample collected from a subject. It will be understood by the skilled artisan that in certain embodiments the level is measured in a sample taken from the subject and may be further manipulated, e.g. through a step of purification, precipitation, separation, and/or centrifugation, and subsequently subjected to a step of determining an protein level, e.g., using mass spectrometric analysis, ELISA, protein immunoprecipitation, immunoelectrophoresis, western blot, and/or immunostaining (e.g., immunofluorescence staining, immunohistochemical staining) with analysis by flow cytometry or microscopy.

In some embodiments, the concentration of expressed therapeutic protein in one or more subjects during a certain period of time is depicted in a concentration/time curve. In exemplary embodiments, the concentration of expressed therapeutic protein is depicted in a plasma concentration/time curve. In other embodiments, the concentration of expressed therapeutic protein is depicted in a tissue concentration/time curve.

Therapeutic threshold: As used herein, the term “therapeutic threshold” refers to the concentration of expressed therapeutic protein that provides the minimum useful therapeutic effect. In a particular embodiment, when the mRNA encodes a secreted protein, the concentration of expressed therapeutic protein is determined in serum. In another embodiment, when the mRNA encodes an intracellular protein, the concentration of expressed therapeutic protein is determined in tissue, e.g., in liver tissue. In another embodiment, when the mRNA encodes an intracellular protein, the concentration of expressed therapeutic protein is determined in a cell population within a tissue, e.g., in liver hepatocytes. In certain embodiments, where the therapeutic protein is an intracellular enzyme, the therapeutic level may be determined indirectly by measuring the effects of the enzyme's activity on the subject's condition.

Methods of determining a therapeutic threshold are known in the art and depend upon the therapeutic polypeptide. In some embodiments, the therapeutic threshold is determined for a therapeutic polypeptide that is an infectious disease antibody, wherein data is collected from subjects having existing immunity to an infectious disease antigen targeted by the antibody, and wherein the therapeutic threshold is determined to be substantially equivalent to the concentration of the infectious disease antibody in the serum of subjects immune to re-infection with the infectious disease. As another example, the therapeutic threshold for a therapeutic intracellular protein, for example, in an enzyme deficiency disorder or disease, can be determined based on data collected from normal human subjects.

Tmax: As used herein, the term “Tmax” refers to the time at which the maximum concentration (Cmax) is observed in serum or a given tissue sample relative to the time of administration. Cmax refers to the maximum (or peak) concentration of a drug after the drug has been administered and prior to administration of a second dose. Specifically, as used herein, the Tmax refers to the time at which the concentration of expressed therapeutic protein is maximal relative to the time of administration of an mRNA encoding the therapeutic protein and prior to a administration of a second dose of the mRNA.

Treating: As used herein, the term “treating” refers to partially or completely alleviating, ameliorating, improving, relieving, delaying onset of, inhibiting progression of, reducing severity of, and/or reducing incidence of one or more symptoms or features of ovarian cancer. For example, “treating” cancer may refer to inhibiting survival, growth, and/or spread of a tumor. Treatment may be measured by reduction in numbers of tumors or reduction in size of a particular tumor and/or reduction in metastasis. Treatment may be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition.

Preventing: As used herein, the term “preventing” refers to partially or completely inhibiting the onset of one or more symptoms or features of a particular infection, disease, disorder, and/or condition.

### Equivalents and Scope

Those skilled in the art will recognize, or be able to ascertain using no more than routine experimentation, many equivalents to the specific embodiments in accordance with the disclosure described herein. The scope of the present disclosure is not intended to be limited to the Description below, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or all of the group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the disclosure, to the tenth of the unit of the lower limit of the range, unless the context clearly dictates otherwise.

All cited sources, for example, references, publications, databases, database entries, and art cited herein, are incorporated into this application by reference, even if not expressly stated in the citation. In case of conflicting statements of a cited source and the instant application, the statement in the instant application shall control.

### Construct Sequences

| | mRNA | ORF Sequence | ORF Sequence | 5′ UTR | 3′ UTR | Construct |
|---|---|---|---|---|---|---|
| | Name | (Amino Acid) | (Nucleotide) | Sequence | Sequence | Sequence |
| SEQ ID | | 1 | 2 | 13 | 14 | 5 |
| NO: | | | | | | |
| | CHIKV- | METDTLLLWVLLLWVPGSTGQV | AUGGAAACCGACACA | GGGAAAU | UGAUAAUA | SEQ ID |
| | 24 | QLVESGGGVVQPGKSLRLSCAA | CUGCUGCUGUGGGUG | AAGAGAG | GGCUGGAG | NO: 5 |
| | heavy | SGFTFRNYGMHWVRQAPGKGLD | CUGCUUCUUUGGGUG | AAAAGAA | CCUCGGUG | consists |
| | chain | WVALISYDGTHKYYKDSLKGRF | CCCGGAUCUACAGGA | GAGUAAG | GCCUAGCU | from 5′ |
| | | TISRDNFQNTVDLQINSLRPDD | CAGGUGCAGCUGGUU | AAGAAAU | UCUUGCCC | to 3′ |
| | | TAVYYCAKELATSGVVEPLDSW | GAAUCUGGCGGCGGA | AUAAGAC | CUUGGGCC | end: 5′ |
| | | GQGTLVTVSSASTKGPSVFPLA | GUUGUGCAGCCUGGC | CCCGGCG | UCCCCCCA | UTR of |
| | | PSSKSTSGGTAALGCLVKDYFP | AAGUCUCUGAGACUG | CCGCCAC | GCCCCUCC | SEQ ID |
| | | EPVTVSWNSGALTSGVHTFPAV | AGCUGUGCCGCCAGC | C | UCCCCUUC | NO: 13, |
| | | LQSSGLYSLSSVVTVPSSSLGT | GGCUUCACCUUCAGA | | CUGCACCC | ORF |
| | | QTYICNVNHKPSNTKVDKKVEP | AACUACGGCAUGCAC | | GUACCCCC | sequence |
| | | KSCDKTHTCPPCPAPELLGGPS | UGGGUCCGACAGGCU | | GUGGUCUU | of SEQ |
| | | VFLFPPKPKDTLMISRTPEVTC | CCAGGCAAAGGCCUU | | UGAAUAAA | ID NO: 2, |
| | | VVVDVSHEDPEVKFNWYVDGVE | GAUUGGGUCGCCCUG | | GUCUGAGU | and 3′ |
| | | VHNAKTKPREEQYNSTYRVVSV | AUCAGCUACGACGGC | | GGGCGGC | UTR of |
| | | LTVLHQDWLNGKEYKCKVSNKA | ACCCACAAGUACUAC | | | SEQ ID |
| | | LPAPIEKTISKAKGQPREPQVY | AAGGACAGCCUGAAG | | | NO: 14 |
| | | TLPPSRDELTKNQVSLTCLVKG | GGCAGAUUCACCAUC | | | |
| | | FYPSDIAVEWESNGQPENNYKT | AGCCGGGACAACUUC | | | |
| | | TPPVLDSDGSFFLYSKLTVDKS | CAGAACACCGUGGAC | | | |
| | | RWQQGNVFSCSVLHEALHSHYT | CUGCAGAUCAACAGC | | | |
| | | QKSLSLSPGK | CUGAGGCCUGACGAC | | | |
| | | | ACCGCCGUGUACUAC | | | |
| | | | UGCGCCAAAGAGCUG | | | |
| | | | GCUACAAGCGGCGUG | | | |
| | | | GUGGAACCUCUGGAU | | | |
| | | | UCUUGGGGACAGGGC | | | |
| | | | ACCCUGGUCACAGUG | | | |
| | | | UCUAGCGCCUCUACA | | | |
| | | | AAGGGACCCAGCGUG | | | |
| | | | UUCCCUCUGGCUCCU | | | |
| | | | AGCAGCAAGAGCACA | | | |
| | | | AGCGGAGGAACAGCC | | | |
| | | | GCUCUGGGCUGUCUG | | | |
| | | | GUCAAGGACUACUUU | | | |
| | | | CCCGAGCCUGUGACC | | | |
| | | | GUGUCCUGGAAUUCU | | | |
| | | | GGCGCUCUGACAUCC | | | |
| | | | GGCGUGCACACCUUU | | | |
| | | | CCAGCUGUGCUGCAA | | | |
| | | | AGCAGCGGCCUGUAC | | | |
| | | | UCUCUGAGCAGCGUC | | | |
| | | | GUGACAGUGCCAAGC | | | |
| | | | AGCUCUCUGGGCACC | | | |
| | | | CAGACCUACAUCUGC | | | |
| | | | AACGUGAACCACAAG | | | |
| | | | CCUAGCAACACCAAG | | | |
| | | | GUGGACAAGAAGGUG | | | |
| | | | GAACCCAAGAGCUGC | | | |
| | | | GACAAGACCCAGACC | | | |
| | | | UGUCCACCCUGUCCU | | | |
| | | | GCUCCAGAACUGCUC | | | |
| | | | GGCGGACCUUCCGUG | | | |
| | | | UUCCUGUUUCCUCCA | | | |
| | | | AAGCCUAAGGACACC | | | |
| | | | CUGAUGAUCAGCAGA | | | |
| | | | ACACCCGAAGUGACC | | | |
| | | | UGCGUGGUGGUGGAC | | | |
| | | | GUGUCUCACGAGGAC | | | |
| | | | CCUGAAGUGAAGUUC | | | |
| | | | AAUUGGUACGUGGAC | | | |
| | | | GGCGUGGAAGUGCAC | | | |
| | | | AACGCCAAGACCAAG | | | |
| | | | CCUAGAGAGGAACAG | | | |
| | | | UACAACAGCACCUAC | | | |
| | | | AGAGUGGUGUCCGUG | | | |
| | | | CUGACCGUGCUGCAC | | | |
| | | | CAGGAUUGGCUGAAC | | | |
| | | | GGCAAAGAGUACAAG | | | |
| | | | UGCAAGGUGUCCAAC | | | |
| | | | AAGGCCCUGCCUGCU | | | |
| | | | CCUAUCGAGAAGACC | | | |
| | | | AUCAGCAAGGCCAAG | | | |
| | | | GGCCAGCCUAGGGAA | | | |
| | | | CCUCAGGUGUACACA | | | |
| | | | CUGCCUCCAAGCAGG | | | |
| | | | GACGAGCUGACCAAG | | | |
| | | | AAUCAGGUGUCCCUG | | | |
| | | | ACCUGCCUCGUGAAG | | | |
| | | | GGCUUCUACCCUUCC | | | |
| | | | GAUAUCGCCGUGGAG | | | |
| | | | UGGGAGAGCAACGGC | | | |
| | | | CAGCCUGAGAACAAC | | | |
| | | | UACAAGACCACUCCU | | | |
| | | | CCUGUGCUGGACAGC | | | |
| | | | GACGGCUCAUUCUUC | | | |
| | | | CUGUACAGCAAGCUG | | | |
| | | | ACAGUGGACAAGUCC | | | |
| | | | AGGUGGCAGCAGGGC | | | |
| | | | AACGUGUUCAGCUGC | | | |
| | | | AGCGUGCUGCACGAA | | | |
| | | | GCCCUGCACAGCCAC | | | |
| | | | UACACCCAGAAGUCC | | | |
| | | | CUGUCUCUGAGCCCU | | | |
| | | | GGCAAA | | | |

CHIKV-24 heavy chain portions

| Signal | | Amino acids 1-20 of | Nucleotides 1- | | | |
|---|---|---|---|---|---|---|
| sequence | | SEQ ID NO: 1 | 60 of SEQ ID | | | |
| | | | NO: 2 | | | |
| Variable | | Amino acids 21-142 of | Nucleotides 61- | | | |
| region (VH) | | SEQ ID NO: 1 | 426 of SEQ ID | | | |
| | | | NO: 2 | | | |
| HCDR1 | | Amino acids 46-53 of | Nucleotides | | | |
| | | SEQ ID NO: 1 | 136-159 of SEQ | | | |
| | | (underlined) | ID NO: 2 | | | |
| | | | (underlined) | | | |
| HCDR2 | | Amino acids 71-78 of | Nucleotides | | | |
| | | SEQ ID NO: 1 | 211-234 of SEQ | | | |
| | | (underlined) | ID NO:2 | | | |
| | | | (underlined) | | | |
| HCDR3 | | Amino acids 117-131 | Nucleotides | | | |
| | | of SEQ ID NO: 1 | 349-393 of SEQ | | | |
| | | (underlined) | ID NO: 2 | | | |
| | | | (underlined) | | | |
| | | Amino acids 143-472 | Nucleotides | | | |
| | | of SEQ ID NO: 1 | 427-1416 of SEQ | | | |
| | | | ID NO: 2 | | | |
| SEQ ID | | 3 | 4 | 13 | 14 | 6 |
| NO: | | | | | | |
| | CHIKV- | METPAQLLFLLLLWLPDTT | AUGGAAACACCCGCU | GGGAAAU | UGAUAAUA | SEQ ID |
| | 24 | GEIVLTQSPGTLSLSPGER | CAGCUGCUGUUCCUG | AAGAGAG | GGCUGGAG | NO: 6 |
| | light | ATLSCRASQSLVSSYFGWY | CUGCUGCUGUGGCUG | AAAAGAA | CCUCGGUG | consists |
| | chain | QQKRGQSPRLLIYAASTRA | CCUGAUACCACAGGC | GAGUAAG | GCCUAGCU | from 5′ |
| | | TGIPDRFSGSGSGTDFTLT | GAGAUCGUGCUGACA | AAGAAAU | UCUUGCCC | to 3′ |
| | | ISRLEPEDFAVYYCQQYGN | CAGAGCCCUGGCACA | AUAAGAC | CUUGGGCC | end: 5′ |
| | | TPFTFGGGTKVEIKRTVAA | CUGUCACUGUCUCCA | CCCGGCG | UCCCCCCA | UTR of |
| | | PSVFIFPPSDEQLKSGTAS | GGCGAAAGAGCCACA | CCGCCAC | GCCCCUCC | SEQ ID |
| | | VVCLLNNFYPREAKVQWKV | CUGAGCUGUAGAGCC | C | UCCCCUUC | NO: 13, |
| | | DNALQSGNSQESVTEQDSK | AGCCAGAGCCUGGUG | | CUGCACCC | ORF |
| | | DSTYSLSSTLTLSKADYEK | UCCAGCUACUUCGGC | | GUACCCCC | sequence |
| | | HKVYACEVTHQGLSSPVTK | UGGUAUCAGCAGAAG | | GUGGUCUU | of SEQ |
| | | SFNRGEC | AGAGGCCAGUCUCCU | | UGAAUAAA | ID NO: 4, |
| | | | CGGCUGCUGAUCUAC | | GUCUGAGU | and 3′ |
| | | | GCCGCUUCUACAAGA | | GGGCGGC | UTR of |
| | | | GCCACCGGCAUUCCC | | | SEQ ID |
| | | | GAUAGAUUCAGCGGC | | | NO: 14 |
| | | | UCUGGCAGCGGCACC | | | |
| | | | GAUUUCACCCUGACA | | | |
| | | | AUCAGCAGACUGGAA | | | |
| | | | CCCGAGGACUUCGCC | | | |
| | | | GUGUACUACUGUCAG | | | |
| | | | CAGUACGGCAACACA | | | |
| | | | CCCUUCACCUUUGGC | | | |
| | | | GGAGGCACCAAGGUG | | | |
| | | | GAAAUCAAGAGAACA | | | |
| | | | GUGGCUGCUCCCAGC | | | |
| | | | GUGUUCAUCUUCCCA | | | |
| | | | CCUUCCGACGAGCAG | | | |
| | | | CUGAAGUCUGGCACA | | | |
| | | | GCCUCUGUCGUGUGC | | | |
| | | | CUGCUGAACAACUUC | | | |
| | | | UACCCUCGGGAAGCC | | | |
| | | | AAGGUGCAGUGGAAG | | | |
| | | | GUGGACAACGCCCUG | | | |
| | | | CAGAGCGGCAACAGC | | | |
| | | | CAAGAGAGCGUGACA | | | |
| | | | GAGCAGGACAGCAAG | | | |
| | | | GACUCCACCUACAGC | | | |
| | | | CUGAGCAGCACACUG | | | |
| | | | ACCCUGAGCAAGGCC | | | |
| | | | GACUACGAGAAGCAC | | | |
| | | | AAGGUGUACGCCUGC | | | |
| | | | GAAGUGACACACCAG | | | |
| | | | GGCCUGUCUAGCCCU | | | |
| | | | GUGACCAAGAGCUUC | | | |
| | | | AACAGAGGCGAGUGC | | | |

CHIKV-24 light chain portions

| | | |
|---|---|---|
| Signal sequence | Amino acids 1-20 of | Nucleotides 1- |
| | SEQ ID NO: 3 | 60 of SEQ ID |
| | | NO: 4 |
| Variable region | Amino acids 21-128 | Nucleotides 61- |
| (VL) | of SEQ ID NO: 3 | 384 of SEQ ID |
| | | NO: 4 |
| LCDR1 | Amino acids 47-53 | Nucleotides |
| | of SEQ ID NO: 3 | 139-159 of SEQ |
| | (underlined) | ID NO: 4 |
| | | (underlined) |
| LCDR2 | Amino acids 71-73 | Nucleotides |
| | of SEQ ID NO: 3 | 211-219 of SEQ |
| | (underlined) | ID NO: 4 |
| | | (underlined) |
| LCDR3 | Amino acids 110-118 | Nucleotides |
| | of SEQ ID NO: 3 | 328-354 of SEQ |
| | (underlined) | ID NO: 4 |
| | | (underlined) |
| Constant region | Amino acids 129-235 | Nucleotides |
| | of SEQ ID NO: 3 | 385-705 of SEQ |
| | | ID NO: 4 |

### Other Embodiments

E1. A method of expressing a therapeutic level of a protein in a human subject in vivo, the method comprising
systemically administering to the human subject a dose of LNP-formulated mRNA, the mRNA encoding the therapeutic protein, optionally at a frequency, effective to achieve a therapeutically-effective level of the therapeutic protein in serum or tissue of the subject.

E2. The method of embodiment 1, wherein the dose is effective to maintain a therapeutically-effective level of the therapeutic protein for a duration.

E3. The method of any one of embodiments 1-2, further comprising administering at least one second dose of the LNP-formulated mRNA to maintain a therapeutically-effective level of the therapeutic protein for a duration.

E4. The method of any one of embodiments 1-3, wherein the therapeutic protein is a secreted protein.

E5. The method of embodiment 4, wherein the protein is an antibody or antigen binding fragment thereof.

E6. The method of embodiment 5, wherein the antibody is a prophylactic antibody, optionally for use in protecting the subject against an infectious disease, optionally wherein the subject is naïve for the infectious disease.

E7. The method of any one of embodiments 5 or 6, wherein the antibody comprises an antibody heavy chain (HC) and an antibody light chain (LC), and wherein the HC and LC are encoded on separate mRNAs.

E8. The method of embodiment 7, wherein the mRNAs encoding the HC and LC are co-formulated in the same LNP, optionally at a 2:1 (HC:LC) w/w ratio.

E9. The method of any one of embodiments 7 or 8, wherein the antibody comprises an engineered single chain antibody, and wherein the HC and LC are encoded by a single mRNA.

E10. The method of any one of embodiments 1-3, wherein the therapeutic protein is an intracellular protein.

E11. The method of any one of embodiments 1-3, wherein the therapeutic protein is a metabolic enzyme.

E12. The method of embodiment 11, wherein the therapeutic protein is a hepatic metabolic enzyme.

E13. The method of any one of embodiments 10-12, wherein the therapeutic protein is expressed in the liver of the subject.

E14. The method of embodiment 13, wherein the therapeutic protein is expressed in hepatocytes of the subject.

E15. The method of any one of the preceding embodiments, wherein the human subject is systemically administered a dose of LNP-formulated mRNA once weekly, once every 2 weeks, once every 3 weeks, once every 4 weeks.

E16. The method of any one of the preceding embodiments, wherein the dose is between 0.1-0.6 mg/kg.

E17. The method of any one of the preceding embodiments, wherein the dose is between 0.2-0.5 mg/kg.

E18. The method of any one of the preceding embodiments wherein the therapeutically-effective level of the therapeutic protein is in the serum of the human subject.

E19. The method of embodiment 18, wherein the therapeutically-effective level is a Cmax at least 2-fold, at least 4-fold, at least 6-fold, at least 8-fold, or at least 10-fold higher than a therapeutic threshold level for the therapeutic protein.

E20. The method of any one of the preceding embodiments wherein the therapeutically-effective level of the therapeutic protein is in a tissue of the human subject.

E21. The method of embodiment 20, wherein the therapeutically-effective level is an increase in expression or bioactivity of the therapeutic protein or a change in the level of a biomarker of the therapeutic protein in a tissue.

E22. A method of protecting a human subject against an infectious disease, the method comprising
systemically administering to the human subject a dose of LNP-formulated mRNA, the mRNA encoding a prophylactic antibody, optionally at a frequency, effective to achieve a therapeutically-effective level of the prophylactic antibody in serum of the subject.

E23. The method of embodiment 22, wherein the subject is naïve for the infectious disease.

E24. The method of any one of embodiments 22 or 23, wherein the prophylactic antibody comprises an antibody heavy chain (HC) and an antibody light chain (LC) comprising amino acid sequences set forth by SEQ ID NOs: 1 and 3 respectively.

E25. The method of embodiment 24, wherein the HC and LC are encoded on separate mRNAs, and wherein the HC and LC comprise nucleotide sequences set forth by SEQ ID NOs: 2 and 4 respectively.

E26. The method of embodiment 25, wherein the mRNAs encoding the HC and LC are co-formulated in the same LNP, optionally at a 2:1 (HC:LC) w/w ratio.

E27. The method of any one of embodiments 22-26, wherein the human subject is systemically administered a dose of LNP-formulated mRNA once weekly, once every 2 weeks, once every 3 weeks, once every 4 weeks.

E28. The method of embodiment 27, wherein the dose is between 0.1-0.6 mg/kg.

E29. The method of embodiment 27, wherein the dose is between 0.2-0.5 mg/kg.

E30. The method of embodiment 29, wherein a therapeutically-effective level is determined by measuring the concentration of the prophylactic antibody in serum collected from the subject.

E31. The method of embodiment 30, wherein the Cmax of the prophylactic antibody in serum is at least 2 μg/mL, at least 3 μg/mL, at least 4 μg/mL, at least 5 μg/mL, at least 6 μg/mL, at least 7 μg/mL, at least 8 μg/mL, at least 9 μg/mL, or at least 10 μg/mL.

E32. The method of embodiment 31, wherein the therapeutically-effective level is a Cmax at least 2-fold, at least 4-fold, at least 6-fold, at least 8-fold, or at least 10-fold higher than a therapeutic threshold level for the prophylactic antibody.

E33. The method of any one of embodiments 29-32, wherein the therapeutically-effective level is maintained for a duration of at least 12 weeks, at least 13 weeks, at least 14 weeks, at least 15 weeks, at least 16 weeks, at least 18 weeks, at least 19 weeks or at least 20 weeks following systemic administration.

E34. The method of any one of embodiments 29-33, wherein the therapeutically-effective levels of the prophylactic antibody in serum are sufficient for neutralization of a target infectious agent.

E35. A method of expressing a therapeutic level of a hepatic metabolic enzyme in a human subject in vivo, the method comprising
systemically administering to the human subject a dose of LNP-formulated mRNA, the mRNA encoding the hepatic metabolic enzyme, optionally at a frequency, effective to achieve a therapeutically-effective level of the hepatic metabolic enzyme in the liver of the subject.

E36. The method of embodiment 35, wherein the human subject is systemically administered a dose of LNP-formulated mRNA once weekly, once every 2 weeks, once every 3 weeks, once every 4 weeks.

E37. The method of embodiment 36, wherein the dose is between 0.1-0.6 mg/kg.

E38. The method of embodiment 37, wherein the therapeutically-effective level of the hepatic metabolic enzyme is in hepatocytes of the human subject.

E39. The method of embodiment 38, wherein the therapeutically-effective level is measured as a change in the level of expression or bioactivity of the hepatic metabolic enzyme or a change in the level of a substrate of the hepatic metabolic enzyme.

E40. The method of any of the preceding embodiments, wherein the LNP-formulated mRNA is systemically administered via intravenous infusion or intravenous injection.

E41. The method of embodiment 40, wherein the LNP-formulated mRNA is systemically administered via intravenous infusion for a duration of 30 minutes to 4 hours.

E42. The method of any one of the preceding embodiments, wherein the human subject is premedicated or co-medicated with a therapeutic agent.

E43. The method of any one of the preceding embodiments, wherein the LNP comprises an ionizable amino lipid, a phospholipid, a cholesterol lipid or cholesterol-derivative lipid, a PEG-lipid or conjugated lipid.

E44. The method of embodiment 43, wherein the ionizable amino lipid is Compound 1.

E45. The method of any one of embodiments 43 or 44 wherein the PEG-lipid is Compound 2.

E46. The method of any one of embodiments 43-45, wherein the LNP comprises 50 mol % ionizable amino lipid and 2 mol % PEG lipid.

E47. The method of embodiment 46, wherein the LNP comprises 50 mol % ionizable amino lipid, 10% DSPC, 38% cholesterol, and 2 mol % PEG lipid.

### EXAMPLES

### Example 1

### Synthesis and Formulation of mRNA Encoding a Fully Human Anti-Chikungunya Antibody

Sequence optimized mRNA encoding human anti-chikungunya antibody heavy chain and light polypeptides were synthesized and prepared. The fully human IgG antibody encoded by the mRNA is CHIKV-24, an antibody originally isolated from B cells of a patient with prior history of potent immunity against chikungunya infection (see, e.g., Kose, N. et al (2019) Science Immunology 4 (35) eaaw6647). The CHIKV-24 antibody binds to the chikungunya envelope E2 protein and has high in vitro neutralizing activity against virus strains from African, Asian, and American viral lineages (EC₅₀ of <10 ng/mL). The heavy and light chains of the encoded antibody were prepared with the variable region amino acid sequences of CHIKV-24 in an IgG1 backbone. The CHIKV-24 heavy chain comprises an amino acid sequence set forth by SEQ ID NO: 1; the CHIKV-24 light chain comprises an amino acid sequence set forth by SEQ ID NO: 3.

The mRNA encoding human anti-chikungunya antibody heavy chain polypeptide was constructed using an ORF sequence identified by SEQ ID NO: 2. The mRNA encoding human anti-chikungunya antibody light chain polypeptide was constructed using an ORF sequence identified by SEQ ID NO: 4. The mRNA sequences included both 5′ and 3′ UTR regions flanking the ORF sequence. The 5′UTR and 3′UTR sequences are set forth in Table 2 below.

TABLE 2
5′UTR and 3′UTR sequences of mRNA encoding
CHIKV-24 heavy or light chain

| | | SEQ | |
|---|---|---|---|
| | | ID | |
| Identifier | Name | NO | Nucleotide sequence |
| 5′UTR | 5′UTR v1.1 | 13 | GGGAAAUAAGAGAGAAAAGAAGA |
| | | | GUAAGAAGAAAUAUAAGACCCCG |
| | | | GCGCCGCCACC |
| 3′UTR | 3′UTR v1.1 | 14 | UGAUAAUAGGCUGGAGCCUCGGU |
| | | | GGCCUAGCUUCUUGCCCCUUGGG |
| | | | CCUCCCCCCAGCCCCUCCUCCCC |
| | | | UUCCUGCACCCGUACCCCCGUGG |
| | | | UCUUUGAAUAAAGUCUGAGUGGG |
| | | | CGGC |

The antibody heavy and light chain mRNA sequences were prepared as modified mRNA. Specifically, during in vitro transcription, modified mRNA were generated using N1-methylpseudouridine-5′-Triphosphate rather than UTP to ensure that the mRNAs contain 100% N1-methyl pseudouridine (m¹ψ) in place of uracil. Further, mRNA were synthesized with a primer that introduces a polyA-tail, and a Cap 1 structure is generated on both mRNAs using Vaccinia Virus Capping Enzyme and a 2′-O methyl-transferase to generate: m7G(5′)ppp(5′)G-2′-O-methyl.

The mRNA encoding heavy or light chains of anti-chikungunya antibody (i.e., CHIKV-24) were co-encapsulated in a lipid nanoparticle at a ratio of 2:1 (HC:LC) by weight. The lipid nanoparticles comprised an ionizable lipid, a sterol, a phospholipid, and a polyethylene glycol (PEG) lipid. The structural components and ratios used to generate the LNPs are shown below in Table 3. The ionizable lipid referred to as Compound 1 and the PEG lipid referred to as Compound 2 are further described herein.

Modified mRNAs encoding the human heavy and light chains of the CHIKV-24 antibody and co-formulated at a 2:1 heavy chain:light chain (HC:LC) w/w ratio in a lipid nanoparticle as described in Table 3 is referred to herein as “LNP-1”. The same mRNAs co-formulated in a Compound 1- and PEG-DMG-containing lipid nanoparticle is referred to as “LNP-2”.

TABLE 3
LNP formulation for delivery of mRNAs encoding CHIKV-24
heavy and light chains

| | Non-cationic | | | |
|---|---|---|---|---|
| Ionizable | helper lipid/ | Structural | | |
| lipid | Phospholipid | lipid | PEG lipid | Ratio |
| (I) | (H) | (S) | (P) | (I:S:H:P) |
| Compound 1 | DSPC | Cholesterol | Compound 2 | 50:10:38:2 |

### Example 2

### Multiple-Dose Study of In Vivo Expression of mRNA Encoding Anti-Chikungunya Virus Antibody in Mice

The prophylactic efficacy of LNP-1 was evaluated in a lethal mouse model of chikungunya infection in AG129 mice. These mice lack IFNα/β and γ receptors and are acutely sensitive to chikungunya infection. To assess the ability of mRNAs encoding the human heavy and light chains of the CHIKV-24 antibody to facilitate protein expression in vivo, mRNAs encoding the heavy and light chains were co-formulated at a 2:1 heavy chain:light chain (HC:LC) w/w ratio, and intravenously administered into AG129 mice via tail vein IV bolus at 0.5 mg/kg, 0.1 mg/kg, or 0.02 mg/kg of total mRNA. Five mice were tested at each dose. The mRNA was formulated in Compound 1- and PEG-DMG-containing lipid nanoparticles LNPs for delivery into the mice (LNP-2). Mice were challenged 24 hours later with Chikungunya virus strain LR06 (LR2006-OPY1, 2C6) at a dose of 10^{2.5} CCID50 by footpad inoculation. Animals were monitored daily for morbidity (e.g., by measuring weight loss) and mortality for up to 21 days after challenge. Control mice were injected with mRNA encoding an antibody that does not bind to chikungunya virus (the CR9114 anti-influenza antibody, as a control antibody). Test and control mice were bled at 24-hours, 48-hours, and 72-hours post-injection to measure total human IgG (huIgG) concentration. Protein was detected using a total human IgG ELISA kit (Abcam, ab100547).

The virus titers in the tissues or plasma of test and control mice were assayed using an infectious cell culture assay where a specific volume of either tissue homogenate or plasma was added to the first tube of a series of dilution tubes. Serial dilutions were made and added to Vero cells. Three days later cytopathic effect (CPE) was used to identify the end-point of infection. Four replicates were used to calculate the 50% cell culture infectious doses per mL of plasma or gram of tissues.

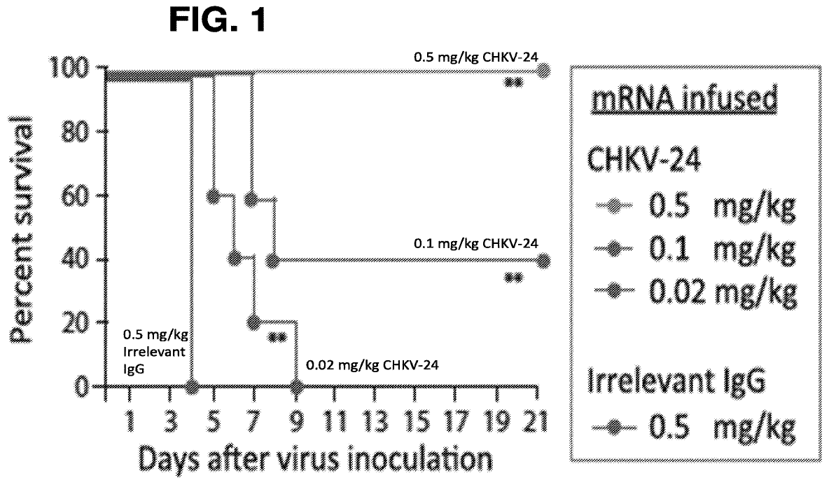

FIG. 1 shows that a dose-responsive improvement in survival of AG129 mice infected with chikungunya virus was observed after treatment with CHIKV-24 mRNA administered intravenously 24 hours prior to virus challenge (**P<0.01, as compared with placebo). 100% of the mice that were administered 0.5 mg/kg of the mRNAs encoding the heavy and light chains of the CHIKV-24 antibody (top line, amounting to a serum concentration of approximately 10 μg/mL), and 40% of the mice that were administered 0.1 mg/kg of the mRNAs encoding the CHIKV-24 antibody (middle line, amounting to a serum concentration of 3 μg/mL) survived for 21 days following challenge with virus. Mice that were administered 0.02 mg/kg of mRNAs encoding the heavy and light chains of CHIKV-24 (bottom line, amounting to 0.5 μg/mL) showed delayed mortality, compared to control mice that were administered mRNA encoding an antibody that does not bind chikungunya virus.

Additionally, mRNA-expressed CHIKV-24 antibody significantly reduced chikungunya virus titers in the serum of AG129 mice at 2 days following virus challenge at all mRNA doses (0.5 mg/kg, 0.1 mg/kg, and 0.02 mg/kg) relative to control mice that were intravenously administered 0.5 mg/kg of mRNA encoding an antibody that does not bind to chikungunya virus (***P<0.001, as compared to placebo, data not shown).

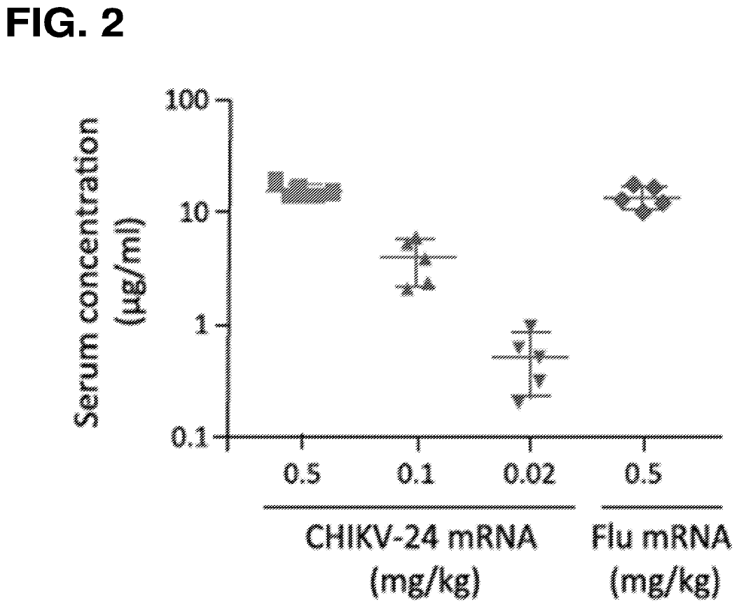

As shown in FIG. 2, administration of increasing amounts of mRNA encoding the CHIKV-24 antibody resulted in greater amounts of antibody in the serum of animals when evaluated post-injection. Serum levels of CHIKV-24 at the 0.5 mg/kg mRNA dose were similar to the same dose of the control (mRNA encoding a control antibody that does not bind to chikungunya virus).

### Example 3

### Multiple-Dose Study of In Vivo Expression of mRNA Encoding Anti-Chikungunya Virus Antibody in Non-Human Primates

To test the expression levels of human CHIKV-24 antibody from modified mRNAs in a nonhuman primate, mRNAs encoding the heavy and light chains of CHIKV-24 were delivered intravenously into cynomolgus monkeys. Either 0.3 mg/kg, 1 mg/kg, or 3 mg/kg of total mRNAs encoding the heavy and light antibody chains, were co-formulated in Compound 1- and Compound 2-containing lipid nanoparticles prior to administration (LNP-1). A total of 4 monkeys were injected at each dose of the CHIKV-24 antibody. The monkeys each received two doses, administered on day 0 and day 168 of the study. The concentration and duration of human antibody was assayed in serum collected from injected monkeys at various time points post-injection using an ELISA assay that detects human antibodies in monkeys (Cayman Chemical Human Therapeutic IgG1 ELISA kit, Item No. 500910, batch #0512236 (pre-bleed) and batch #0512421 (all time points post injection)).

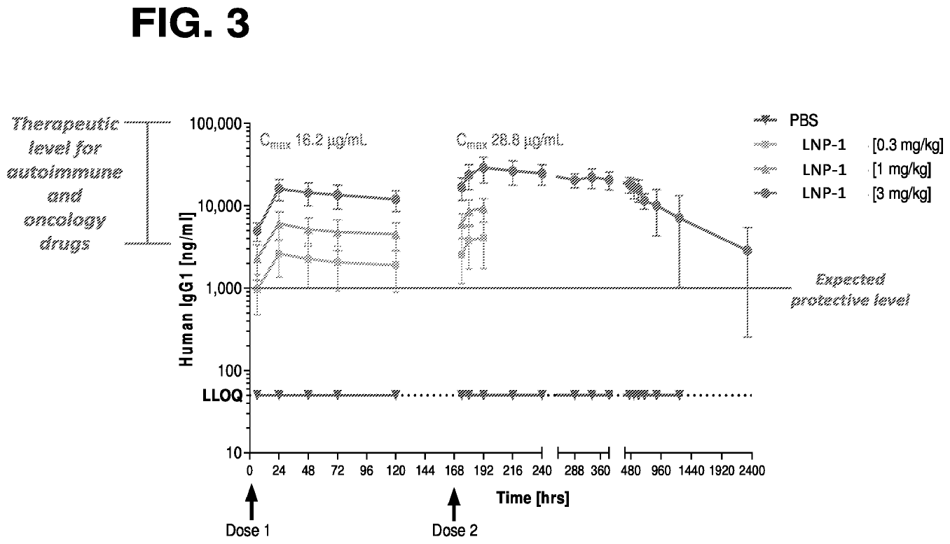

As shown in FIG. 3, the CHIKV-24 antibody was expressed from modified mRNAs injected in monkeys over the course of greater than 2000-hours post-injection. The Cmax of mRNAs injected at a dose of 3 mg/kg (top line) was approximately 16 μg/mL and 29 μg/mL for the CHIKV-24 antibody following the first and second dose respectively. The Cmax was measured at 24 hours following administration of each dose of mRNA. Antibody levels expected to be protective were maintained throughout the duration of the study for the 3 mg/kg dose. The CHIKV-24 antibody was expressed from modified mRNAs at lower concentration levels over the course of the study following injection of mRNAs at the 1 or 0.3 mg/kg dose.

These results demonstrate that therapeutic levels of CHIKV-24 expected to be protective can be achieved in the serum following multiple doses of modified mRNA encoding CHIKV-24 heavy and light chains.

### Example 4

### Evaluation of Safety and Activity of mRNA Encoding Anti-Chikungunya Virus Antibody in Humans

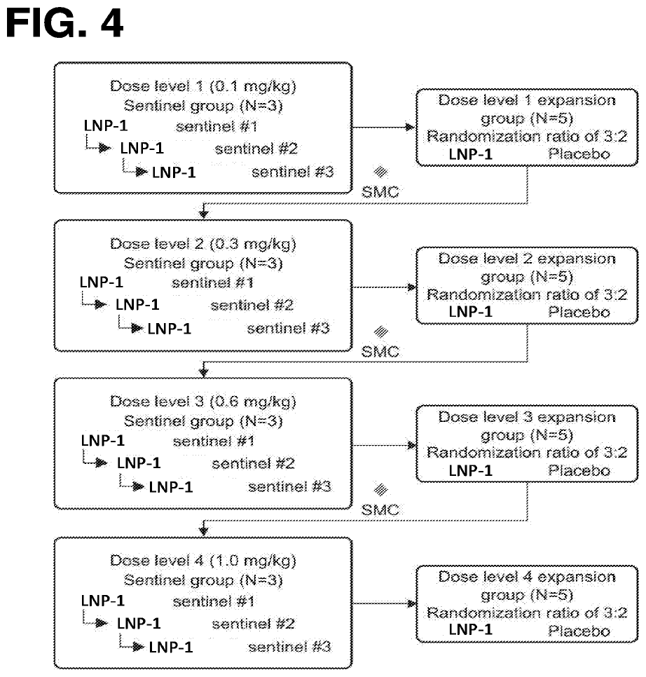

As shown in FIG. 4, a randomized, placebo-controlled Phase 1 study was designed to evaluate the safety and tolerability of up to four escalating doses (0.1, 0.3, 0.6 and 1 mg/kg of total mRNA) of LNP-1 administered via intravenous infusion to healthy adults. Secondary objectives were to determine the pharmacology of LNP-1 and to evaluate whether the antibodies produced would fold properly into functional antibodies that were then excreted into the serum and could bind and neutralize chikungunya virus in vitro, thereby confirming the potential for passive immunization of individuals via the production of functional circulating antibody. Passive immunity provides transient but rapid protection against an infectious disease and is particularly important when immediate protection is needed, such as in a pandemic setting.

### Study Design

As shown in FIG. 4, a sentinel dosing strategy was employed for each cohort. A safety group of three subjects were enrolled, all of which received LNP-1, with a staggered minimum 7-day interval between each subject prior to administering the dose to remaining subjects in the dose cohort. Following a confirmation of the safety data for the sentinel subjects of each cohort, the remainder of the dose level cohort was enrolled in the study. The remaining 5 subjects within each cohort were randomly assigned in a 3:2 ratio to receive LNP-1 or placebo.

The initial analysis evaluated the safety and activity of intravenous administration of LNP-1 at three dose levels of 0.1, 0.3 and 0.6 mg/kg. The study enrolled a total of 22 healthy adults. Of these, 6 subjects received the 0.1 mg/kg dose, 6 received the 0.3 mg/kg dose, and 4 received the 0.6 mg/kg dose. A total of 6 subjects received placebo treatment. All participants in the study received antihistamine premedication. No participants received corticosteroids either as pre-medication or treatment. For each cohort, LNP-1 was supplied in frozen vials containing a 1.4 mL fill volume at a concentration of 2 mg/mL formulated with 20 mM Tris buffer, 60 mM NaCl, 8% sucrose, 1.3% ethanol, and 1 mM diethylenetriaminepentaacetic acid at pH 7.5. The placebo was 0.9% sodium chloride obtained from a commercial vendor. LNP-1 was administered on Day 1 as a single intravenous infusion in a volume of 100 mL over 1 hour using a controlled infusion device. The infusion time was further extended up to 4 hours in the event of an infusion reaction or the occurrence of an adverse reaction assessed as related to infusion of LNP-1 experienced by any subject in a given cohort.

### Safety and Pharmacology Assessment

Subjects were observed at the study site for safety assessment and pharmacokinetic/pharmacodynamic (PK/PD) sampling for 48 hours following completion of dosing on Day 1. Adverse events were captured through the study follow-up period of 12 months and additional plasma PK samples were collected through the end of the study (Week 52; 12 months). PD assessments included collection of blood samples for the determination of serum CHIKV-24 IgG concentrations. Blood samples were collected for all subjects within 60 minutes prior to LNP-1 infusion, at mid-infusion, at end of infusion, at 2, 4, 6, 8, 12, 18, 24, 36, and 48 hours post-infusion, on Days 7, 14, 21, and 28 post-infusion, and on weeks 8, 12, 24, 36, 48, and 52 post-infusion. Serum CHIKV-24 IgG concentration was determined by ELISA.

Safety and tolerability was be assessed by the following endpoints: monitoring and recording of adverse events (AEs), prior and concomitant medication, clinical laboratory test results (hematology, coagulation, serum chemistry including liver enzymes, and urinalysis), vital sign measurements (systolic and diastolic blood pressure, heart rate, respiratory rate, and body temperature), ECG results (and cardiac enzymes when obtained per protocol), and physical examination findings.

An AE was defined as any untoward medical occurrence in a subject administered the study drug (i.e., LNP-1) and which did not necessarily have a causal relationship with receipt of the study drug. An AE could therefore be any unfavorable and unintended sign (including an abnormal laboratory finding), symptom, or disease temporally associated with the use of either the study drug, a study product, and/or a study procedure(s). Subjects were instructed to contact the investigator at any time after study drug infusion if any symptoms developed. AEs were assessed from the time of informed consent until end of study/early termination and were followed until they resolved, stabilized, or were judged by the investigator to be not clinically significant. If any doubt as to whether a clinical observation was an AE, the event was reported.

The severity (or intensity) of an AE referred to the extent to which it affected a subject's daily activities and were classified according to the toxicity grading scale as mild (grade 1), moderate (grade 2), severe (grade 3), and potentially life threatening (grade 4) using the following criteria: (i) Mild (grade 1) were events classified as not interfering with a subject's daily activities; (ii) Moderate (grade 2) were events classified as causing some interference with a subject's daily activities, but not requiring medical intervention; (iii) Severe (grade 3) were events classified as preventing a subject's daily activities and require medical intervention; and (iv)Life threatening (grade 4): were events classified as requiring an emergency room visit or hospitalization. The CTCAE v5 (National Cancer Institute Common Terminology Criteria for Adverse Events Version 5 (CTCAE v5, November 2017)) grading scale was used to assess all AEs and laboratory abnormalities. Vital signs were graded according to “Guidance for industry—Toxicity grading scale for healthy adult and adolescent volunteers enrolled in preventative vaccine clinical trials”, Tables for clinical abnormalities (DHHS 2007).

An AE was considered “serious” if it resulted in any of the following outcomes: (i) death; (ii) put the subject at risk of death at the time of the event; (iii) required inpatient hospitalization or prolonged of existing hospitalization; (iv) resulted in persistent or significant disability/incapacity; (v) was a congenital anomaly/birth defect; (vii) was a medically important event deemed as events that may not result in death, be life threatening, or require hospitalization but may be considered serious when, based upon appropriate medical judgment, they jeopardize the subject and may require medical or surgical intervention to prevent an outcome listed in (i)-(v). For example, an allergic bronchospasm that requires intensive treatment in an emergency room or at home, blood dyscrasias or convulsions that do not result in inpatient hospitalization, or the development of drug dependency or drug abuse.

### Study Evaluation

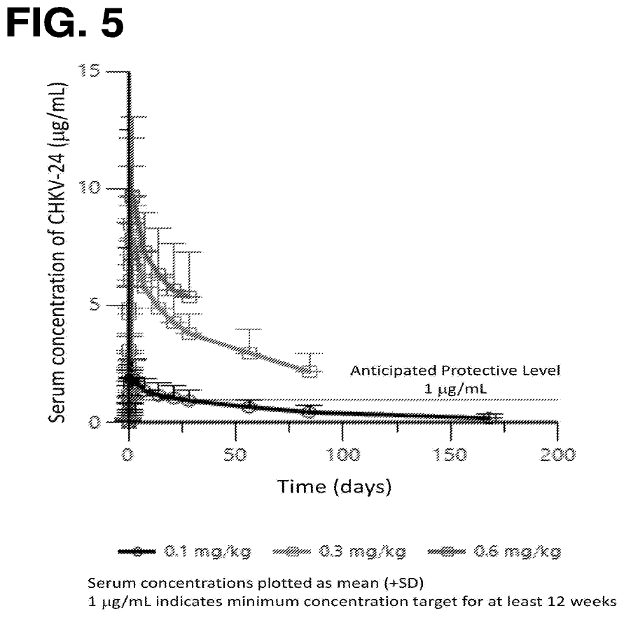

As shown in FIG. 5, administration of LNP-1 resulted in dose-related increases in CHIKV-24 antibody levels in serum. The average maximum observed serum concentration (Cₘₐₓ) antibody levels of 2.0, 7.9 and 10.2 ug/mL were measured at the low, middle and high doses, respectively. At all doses, all participants exceeded the levels of antibody expected to be protective against chikungunya infection (>1 μg/mL) following a single dose, with the middle dose projected to maintain antibody levels above protective levels for at least 16 weeks.

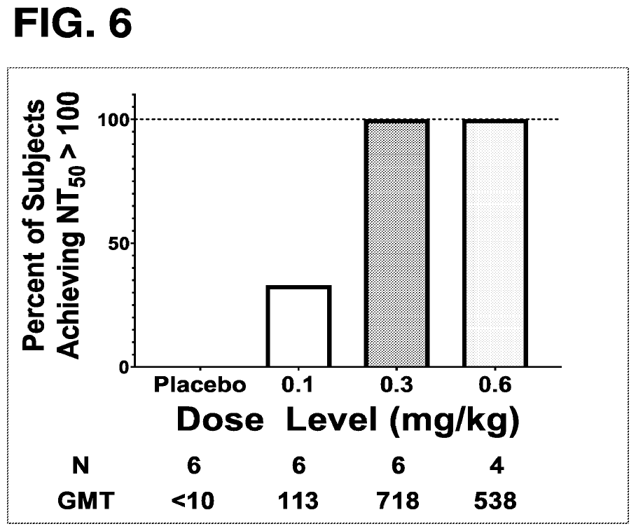

As shown in FIG. 6, participants also showed circulating neutralizing antibody activity against chikungunya virus replication. Neutralizing titers were assessed using an NT50 assay as described in Example 2. While placebo resulted in neutralizing titers below the lower limit of detection, neutralizing antibody titers were observed at all dose levels for subjects treated with LNP-1. Neutralizing antibody titers were measured by endpoint titration and calculated as 50% neutralization (NT₅₀). Additionally, treatment with 0.3 and 0.6 mg/kg doses of LNP-1 resulted in all subjects having NT₅₀ exceeding 100. The average serum CHKV-24 IgG antibody titer measured as a geometric mean titer (GMT) was 113, 718, and 538 for cohort dosed at 0.1 mg/kg, 0.3 mg/kg, and 0.6 mg/kg respectively, while the placebo GMT was <10. Thus, LNP-1 resulted in the production of fully functional protein in vivo.

Moreover, LNP-1 was well-tolerated at the 0.1 and 0.3 mg/kg doses with no significant adverse events (AEs). Three of the four participants at the 0.6 mg/kg dose level had infusion related reactions, with the highest grade by subject being Grade 1 (one participant), Grade 2 (one participant) and Grade 3 (one participant). The Grade 3 adverse events were tachycardia and an elevated white blood cell count. The fourth participant at 0.6 mg/kg had no related adverse events. There were no serious adverse events in the study. All adverse events were transient and resolved spontaneously without treatment.

These data demonstrate safety and activity in a Phase 1 study evaluating escalating doses of LNP-1 administered via intravenous infusion in healthy adults. At all three dose levels, the administration of LNP-1 led to detectable levels of CHIKV-24 antibody in all participants, ranging from 1 μg/mL to 14 μg/mL.

### Example 5

### LNP-1 Provides a Dose-Dependent Increase in Circulating Levels of Antibody

This example further describes the randomized, placebo-controlled Phase 1 study introduced in Example 4, that was updated to include additional cohorts for assessing the safety and tolerability of LNP-1 in healthy adult subjects.

Briefly, the study design as shown in FIG. 4 was updated to include 6 dose level cohorts and 1 split dose cohort. The dose level cohorts described in Example 4 were updated as follows. Cohorts 1, 2, and 3 received LNP-1 doses of 0.1, 0.3, and 0.6 mg/kg respectively as described in Example 4, and were dosed without dexamethasone in the premedication regimen. Cohort 4 was updated to be a 0.45 mg/kg dose level, and was designed to be an optional cohort. Cohort 5 was added at the 0.6 mg/kg dose level and cohort 6 was added at the 1.0 mg/kg dose level.

Due to the infusion related AEs observed following dosing of cohort 3 as described in Example 4, a dexamethasone premedication regimen was added for optional cohort 4, cohort 5, and cohort 6. The regimen included 10 mg dexamethasone administered by intravenous injection approximately 90 minutes prior to LNP-1 administration. Cohort 7 was added and designed to receive a repeat dose of LNP-1 administered once weekly for two weeks. Specifically, the cohort included administration of two infusions of LNP-1 at the 0.3 mg/kg dose level, with the first infusion administered on Day 1 and the second infusion administered on Day 8.

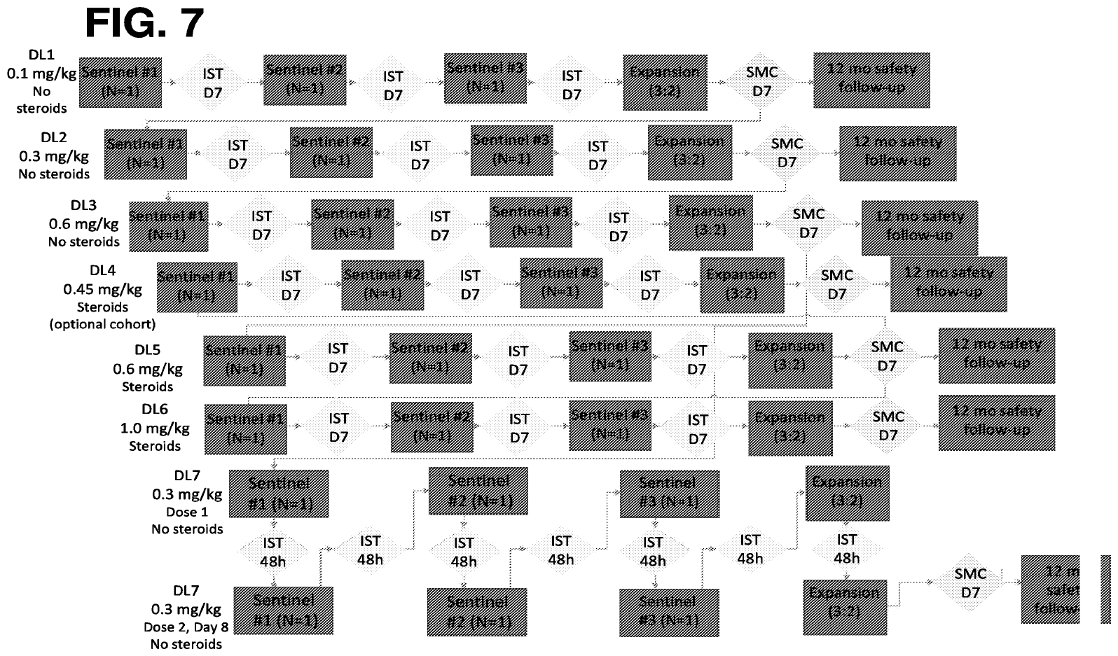

The updated study design is shown in FIG. 7. As described in Example 4, the study design included eight subjects per cohort. Three sentinel subjects were administered LNP-1, with an intervening safety evaluation between each. Following approval of the safety performance for the sentinel subjects, dosing proceeded to an expansion group of 5 subjects randomly assigned to receive LNP-1 (3 subjects) or placebo infusion of 0.9% sodium chloride (2 subjects).

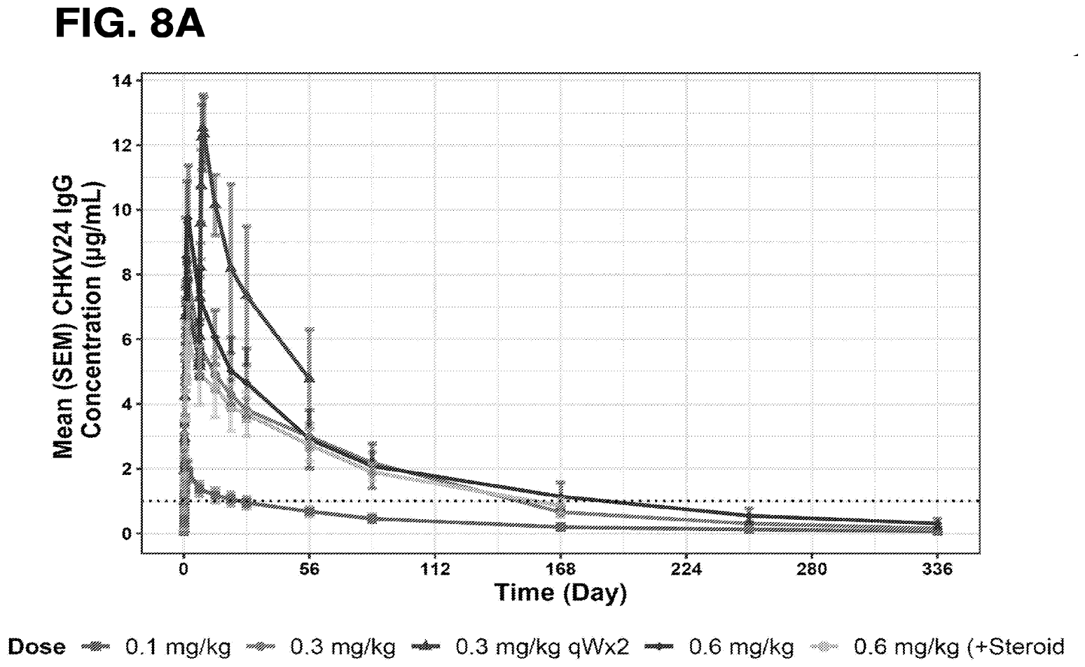

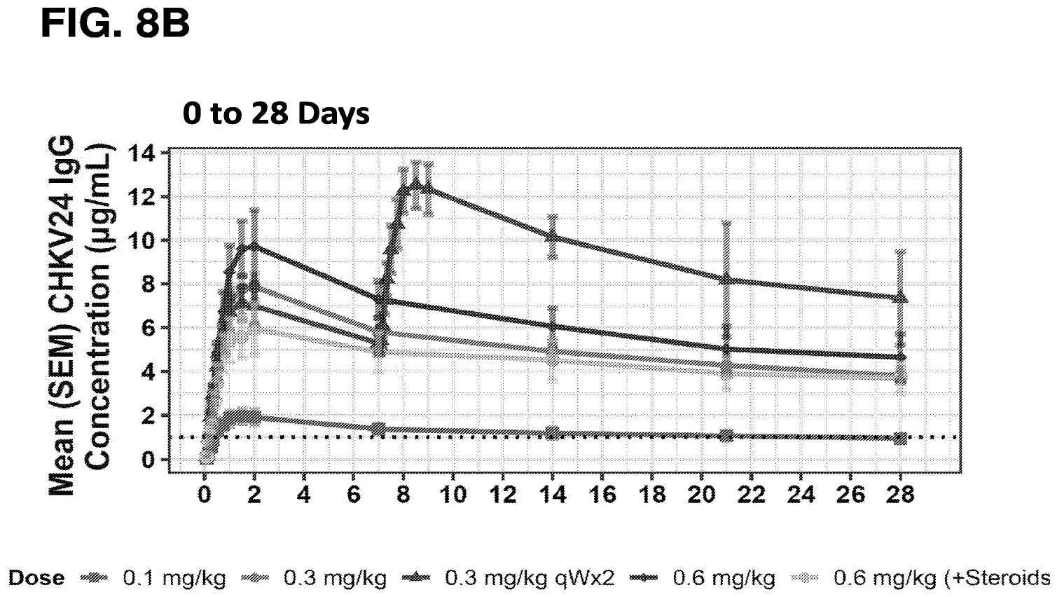

According to the updated study design, a total of five cohorts were investigated in a sequential dose-escalation manner, including cohorts 1-3 as described in Example 4, and cohort 5 and 7 as described above. Study objectives were evaluated as described in Example 4, and included safety and pharmacology endpoints. Briefly the following baseline corrected serum PD parameters were calculated as endpoints for CHIKV-24 IgG concentration based on the actual sampling times relative to the start of infusion: maximum observed effect (Eₘₐₓ); time to the maximum observed effect (TEₘₐₓ); half-life (t_{1/2}); area under the effect curve (AUEC) from time 0 to 7 days (AUEC₀₋₇); and area under the effect curve (AUEC) from time 0 to the last measurable concentration (AUECₗₐₛₜ). Quantification of the PD parameters is shown in Table 4. The average serum CHIKV-24 IgG concentration measured for each cohort over the study duration and through day 28 is shown in FIG. 8A and FIG. 8B respectively.

TABLE 4
CHIKV-24 IgG PD Parameters

| | Dose | | | TEmax | Emax | AUEC0-last | AUEC0-7 |
|---|---|---|---|---|---|---|---|
| Cohort | Number | | t1/2 (h) | (h) | (μg/mL) | (h*μg/mL) | (h*μg/mL) |
| COHORT 1 | 1 | Mean | 1470 | 36 | 1.97 | 2730 | 267 |
| (0.1 mg/kg) | | CV % | 34.3 | 21.1 | 40.4 | 58 | 40 |
| | | Min | 610 | 24 | 1.1 | 1070 | 154 |
| | | Max | 2010 | 48 | 3.1 | 5480 | 420 |
| COHORT 2 | 1 | Mean | 1940 | 46 | 7.88 | 11300 | 1090 |
| (0.3 mg/kg) | | CV % | 16.4 | 10.6 | 18.2 | 24.1 | 21.4 |
| | | Min | 1420 | 36 | 6.28 | 7720 | 870 |
| | | Max | 2290 | 48 | 9.97 | 15400 | 1420 |
| COHORT 3 | 1 | Mean | 1550 | 45 | 10.2 | 12900 | 1340 |
| (0.6 mg/kg) | | CV % | 50.4 | 13.3 | 29.6 | 55.9 | 22 |
| | | Min | 418 | 36 | 7.01 | 5070 | 957 |
| | | Max | 2210 | 48 | 14.2 | 22500 | 1650 |
| COHORT 5 | 1 | Mean | 1900 | 42 | 6.05 | 7560 | 856 |
| (0.6 mg/kg + | | CV % | 10.9 | 23.9 | 47 | 33.8 | 46.9 |
| Steroids) | | Min | 1590 | 24 | 2.34 | 4330 | 356 |
| | | Max | 2080 | 48 | 10.5 | 11800 | 1490 |
| COHORT 7 | 1 | Mean | NR | 36 | 7.17 | 988 | 988 |
| (0.3 mg/kg | | CV % | NR | 21.1 | 26.9 | 28.6 | 28.6 |
| qWx2) | | Min | NR | 24 | 4.01 | 538 | 538 |
| | | Max | NR | 48 | 9.66 | 1400 | 1400 |
| | 2 | Mean | 1075 | 40 | 12.9 | 4120 | 1860 |
| | | CV % | NR | 24.5 | 21.4 | 89.5 | 21.9 |
| | | Min | 1100 | 24 | 8.19 | 1770 | 1190 |
| | | Max | 1050 | 48 | 16.7 | 10900 | 2430 |

As shown in FIGS. 8A-8B and Table 4, administration of LNP-1 resulted in a dose-related increase in CHIKV-24 IgG exposure levels. Specifically, the average AUEC₀₋₇ for cohort 1 (dose level 0.1 mg/kg), cohort 2 (dose level 0.3 mg/kg), and cohort 3 (dose level 0.6 mg/kg) was 267, 1090, and 1340 h*μg/mL respectively.

A single 0.3 mg/kg dose (cohort 2) resulted in CHKV-24 IgG levels that exceeded the 1 μg/mL target concentration for at least 16 weeks following administration. The CHKV-24 IgG half-life (t_{1/2}) was estimated to be approximately 72 days. Together these results indicate the single 0.3 mg/kg dose level resulted in a concentration level of CHKV-24 IgG expected to provide protection against chikungunya infection for several months following LNP-1 administration.

Additionally, a similar level of CHKV-24 IgG exposure was observed following the first 0.3 mg/kg dose for both cohort 2 and cohort 7, indicating consistency in CHKV-24 IgG expression following administration of equivalent doses of LNP-1. Specifically, the average AUEC₀₋₇ for cohort 2 and cohort 7 was 1090 and 988 h*μg/mL respectively following the first dose. Additionally, administration of the second 0.3 mg/kg dose for cohort 7 resulted in an approximately 1.8-fold increase in CHKV-24 IgG exposure compared to the first dose. The average AUEC₀₋₇ following the first and second dose was 988 and 1860 h*μg/mL respectively, indicating a significant boost in CHKV-24 IgG expression with administration of the second LNP-1 dose.

Premedication with dexamethasone resulted in a decrease in CHIKV-24 IgG exposure. Specifically, cohort 5 which received dexamethasone premedication had an approximately 1.6-fold decrease in CHIKV-24 IgG exposure compared to cohort 3, which received an equivalent dose of LNP-1 (average AUEC₀₋₇ of 856 and 1340 h*μg/mL respectively). However, the CHIKV-24 IgG expression level for cohort 5 was still well above the 1 μg/mL target concentration for at least 16-weeks following administration and a t_{1/2} of approximately 72 days.

Safety endpoints were evaluated as described in Example 4. Across all cohorts evaluated, gastrointestinal AEs were the most common, and included nausea, decreased appetite, and emesis. Also reported were headache, dizziness, chills and generalized muscle aches AEs. Most of the events were Grade 1 (mild, 22 events); with three of the GI events being Grade 2 (moderate). The split dose cohort (cohort 7) experienced similar AE profile to the previous cohorts (cohorts 1, 2, 3, 5), and there was no exacerbation or worsening of symptoms in any one subject following receipt of the second dose as compared to following receipt of the first dose. Importantly, all AEs were transient and resolved spontaneously without treatment, and no serious AEs were reported. Additionally, no meaningful changes were observed in liver or kidney laboratory results.

Overall, these data demonstrate that administration of LNP-1 via intravenous infusion, particularly at the 0.3 mg/kg and 0.6 mg/kg dose levels, resulted in a safe and durable expression of CHIKV-24 IgG at levels expected to provide protection against chikungunya infection. Moreover, the safety and increased CHKV-24 IgG production based on the two-dose regimen (cohort 7) demonstrates the platform's ability for repeat dosing.

What is claimed:

1. A method of producing a therapeutic level of an antibody in a human subject in vivo, the method comprising systemically administering to the subject an LNP-formulated mRNA, the mRNA encoding the antibody, in a dose effective to produce a Cmax of at least 1 μg/mL of the antibody in serum of the subject.

2. The method of claim 1, wherein the Cmax is at least 2 μg/mL, at least 6 μg/mL, or at least 10 μg/mL.

3. The method of claim 1 or 2, wherein the antibody has a half-life of at least 50-100 days.

4. The method of any one of claims 1-3, further comprising systemically administering a second dose of the LNP-formulated mRNA within about 1 week, about 2 weeks, about 3 weeks, or about 4 weeks following the first dose.

5. The method of any one of claims 1-4, wherein the Cmax following the second dose is equal to or greater than the Cmax following the first dose.

6. The method of claim 5, wherein the Cmax following the second dose is at least 1.1-fold, 1.4-fold, or 1.8-fold greater than the Cmax following the first dose.

7. The method of any one of claims 1-6, wherein the dose is between 0.1-0.6 mg/kg.

8. The method of claim 7, wherein the dose is 0.1, 0.3, 0.45, or 0.6 mg/kg.

9. The method of any one of claims 1-8, wherein the therapeutic level of the antibody in serum is maintained for a duration following systemic administration.

10. The method of claim 9, wherein the duration is at least 12 weeks, at least 13 weeks, at least 14 weeks, at least 15 weeks, at least 16 weeks, at least 18 weeks, at least 19 weeks, at least 20 weeks, at least 21 weeks, at least 22 weeks, at least 23 weeks, or at least 24 weeks.

11. The method of any one of claims 1-10, wherein the antibody comprises an antibody heavy chain (HC) and an antibody light chain (LC), and wherein the HC and LC are encoded on separate mRNAs.

12. The method of claim 11, wherein the mRNAs encoding the HC and LC are co-formulated in the same LNP, optionally at a 2:1 (HC:LC) w/w ratio.

13. The method of claim 11 or 12, wherein when the LNP-formulated mRNA is systemically administered, the HC and LC are expressed from separate mRNAs and combined to form the antibody.

14. The method of any one of claims 1-10, wherein the antibody comprises an engineered single chain antibody, and wherein the HC and LC are encoded by a single mRNA.

15. A method of producing a therapeutic level of an antibody in a human subject in vivo, the method comprising systemically administering to the subject an LNP-formulated mRNA, the mRNA encoding the antibody, in a first dose effective to produce a Cmax of at least 1 μg/mL of the antibody in serum of the subject, and at least one second dose effective to produce a Cmax equal to or greater than a Cmax following the first dose.

16. The method of claim 15, wherein the Cmax following the second dose is at least 1.1-fold, 1.4-fold, or 1.8-fold greater than the Cmax following the first dose.

17. The method of claim 15 or 16, wherein the Cmax following the first dose is at least 2 μg/mL, at least 6 μg/mL, or at least 10 μg/mL.

18. The method of any one of claims 15-17, wherein the first and second dose of LNP-formulated mRNA is between 0.1-0.6 mg/kg.

19. The method of any one of claims 15-18, wherein the second dose is administered once weekly, once every 2 weeks, once every 3 weeks, or once every 4 weeks.

20. The method of any one of claims 15-19, wherein the therapeutic level of the antibody in serum is maintained for a duration following systemic administration.

21. The method of claim 20, wherein the duration is at least 12 weeks, at least 13 weeks, at least 14 weeks, at least 15 weeks, at least 16 weeks, at least 18 weeks, at least 19 weeks, at least 20 weeks, at least 21 weeks, at least 22 weeks, at least 23 weeks, or at least 24 weeks.

22. The method of any one of the preceding claims, wherein the antibody is a prophylactic antibody, optionally for use in protecting the subject against an infectious disease, optionally wherein the subject is naive for or uninfected by the infectious disease.

23. The method of claim 22, wherein the therapeutic level of the prophylactic antibody in serum is sufficient to neutralize a target infectious agent.

24. The method of any one of the preceding claims, wherein the LNP comprises an ionizable amino lipid, a phospholipid, a cholesterol lipid or cholesterol-derivative lipid, a PEG-lipid or conjugated lipid.

25. The method of claim 24, wherein the ionizable amino lipid is Compound 1.

26. The method of claim 24 or 25, wherein the PEG-lipid is Compound 2.

27. The method of any one of claims 24-26, wherein the LNP comprises 20-60 mol % ionizable amino lipid and 0.5-15 mol % PEG lipid.

28. The method of any one of claims 24-27, wherein the LNP comprises 20-60 mol % ionizable amino lipid, 5-25% DSPC, 25-55% cholesterol, and 0.5-15 mol % PEG lipid.

29. The method of any one of the preceding claims, wherein the LNP-formulated mRNA is systemically administered via intravenous infusion or intravenous injection.

30. The method of any one of claims 1-29, wherein the LNP and mRNA are formulated in the same vial.

31. The method of any one of claims 1-29, wherein the LNP and mRNA are formulated in separate vials.

32. The method of claim 31, wherein the LNP and mRNA are combined prior to being systemically administered.

33. The method of any one of claims 1-32, wherein the LNP-formulated mRNA is systemically administered via intravenous infusion for a duration of 30 minutes to 4 hours.


SEQUENCE LISTING (16 sequences)

SEQ ID NO: 1 - protein, 472 aa, Artificial Sequence
  Synthetic: CHIKV-24 heavy chain ORF Sequence (Amino Acid)
METDTLLLWVLLLWVPGSTGQVQLVESGGGVVQPGKSLRLSCAASGFTFRNYGMHWVRQAPGKGLDWVALISYDGTHKYYKDSLKGRFTISRDNFQNTVDLQINSLRPDDTAVYYCAKELATSGVVEPLDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 2 - RNA, 1416 nt, Artificial Sequence
  Synthetic: CHIKV-24 heavy chain ORF Sequence (Nucleotide)
auggaaaccgacacacugcugcugugggugcugcuucuuugggugcccggaucuacaggacaggugcagcugguugaaucuggcggcggaguugugcagccuggcaagucucugagacugagcugugccgccagcggcuucaccuucagaaacuacggcaugcacuggguccgacaggcuccaggcaaaggccuugauugggucgcccugaucagcuacgacggcacccacaaguacuacaaggacagccugaagggcagauucaccaucagccgggacaacuuccagaacaccguggaccugcagaucaacagccugaggccugacgacaccgccguguacuacugcgccaaagagcuggcuacaagcggcgugguggaaccucuggauucuuggggacagggcacccuggucacagugucuagcgccucuacaaagggacccagcguguucccucuggcuccuagcagcaagagcacaagcggaggaacagccgcucugggcugucuggucaaggacuacuuucccgagccugugaccguguccuggaauucuggcgcucugacauccggcgugcacaccuuuccagcugugcugcaaagcagcggccuguacucucugagcagcgucgugacagugccaagcagcucucugggcacccagaccuacaucugcaacgugaaccacaagccuagcaacaccaagguggacaagaagguggaacccaagagcugcgacaagacccacaccuguccacccuguccugcuccagaacugcucggcggaccuuccguguuccuguuuccuccaaagccuaaggacacccugaugaucagcagaacacccgaagugaccugcguggugguggacgugucucacgaggacccugaagugaaguucaauugguacguggacggcguggaagugcacaacgccaagaccaagccuagagaggaacaguacaacagcaccuacagagugguguccgugcugaccgugcugcaccaggauuggcugaacggcaaagaguacaagugcaagguguccaacaaggcccugccugcuccuaucgagaagaccaucagcaaggccaagggccagccuagggaaccucagguguacacacugccuccaagcagggacgagcugaccaagaaucaggugucccugaccugccucgugaagggcuucuacccuuccgauaucgccguggagugggagagcaacggccagccugagaacaacuacaagaccacuccuccugugcuggacagcgacggcucauucuuccuguacagcaagcugacaguggacaaguccagguggcagcagggcaacguguucagcugcagcgugcugcacgaagcccugcacagccacuacacccagaagucccugucucugagcccuggcaaa

SEQ ID NO: 3 - protein, 235 aa, Artificial Sequence
  Synthetic: CHIKV-24 light chain ORF Sequence (Amino Acid)
METPAQLLFLLLLWLPDTTGEIVLTQSPGTLSLSPGERATLSCRASQSLVSSYFGWYQQKRGQSPRLLIYAASTRATGIPDRFSGSGSGTDFTLTISRLEPEDFAVYYCQQYGNTPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - RNA, 705 nt, Artificial Sequence
  Synthetic: CHIKV-24 light chain ORF Sequence (Nucleotide)
auggaaacacccgcucagcugcuguuccugcugcugcuguggcugccugauaccacaggcgagaucgugcugacacagagcccuggcacacugucacugucuccaggcgaaagagccacacugagcuguagagccagccagagccugguguccagcuacuucggcugguaucagcagaagagaggccagucuccucggcugcugaucuacgccgcuucuacaagagccaccggcauucccgauagauucagcggcucuggcagcggcaccgauuucacccugacaaucagcagacuggaacccgaggacuucgccguguacuacugucagcaguacggcaacacacccuucaccuuuggcggaggcaccaagguggaaaucaagagaacaguggcugcucccagcguguucaucuucccaccuuccgacgagcagcugaagucuggcacagccucugucgugugccugcugaacaacuucuacccucgggaagccaaggugcaguggaagguggacaacgcccugcagagcggcaacagccaagagagcgugacagagcaggacagcaaggacuccaccuacagccugagcagcacacugacccugagcaaggccgacuacgagaagcacaagguguacgccugcgaagugacacaccagggccugucuagcccugugaccaagagcuucaacagaggcgagugc

SEQ ID NO: 5 - RNA, 1592 nt, Artificial Sequence
  Synthetic: Construct Sequence
gggaaauaagagagaaaagaagaguaagaagaaauauaagaccccggcgccgccaccauggaaaccgacacacugcugcugugggugcugcuucuuugggugcccggaucuacaggacaggugcagcugguugaaucuggcggcggaguugugcagccuggcaagucucugagacugagcugugccgccagcggcuucaccuucagaaacuacggcaugcacuggguccgacaggcuccaggcaaaggccuugauugggucgcccugaucagcuacgacggcacccacaaguacuacaaggacagccugaagggcagauucaccaucagccgggacaacuuccagaacaccguggaccugcagaucaacagccugaggccugacgacaccgccguguacuacugcgccaaagagcuggcuacaagcggcgugguggaaccucuggauucuuggggacagggcacccuggucacagugucuagcgccucuacaaagggacccagcguguucccucuggcuccuagcagcaagagcacaagcggaggaacagccgcucugggcugucuggucaaggacuacuuucccgagccugugaccguguccuggaauucuggcgcucugacauccggcgugcacaccuuuccagcugugcugcaaagcagcggccuguacucucugagcagcgucgugacagugccaagcagcucucugggcacccagaccuacaucugcaacgugaaccacaagccuagcaacaccaagguggacaagaagguggaacccaagagcugcgacaagacccacaccuguccacccuguccugcuccagaacugcucggcggaccuuccguguuccuguuuccuccaaagccuaaggacacccugaugaucagcagaacacccgaagugaccugcguggugguggacgugucucacgaggacccugaagugaaguucaauugguacguggacggcguggaagugcacaacgccaagaccaagccuagagaggaacaguacaacagcaccuacagagugguguccgugcugaccgugcugcaccaggauuggcugaacggcaaagaguacaagugcaagguguccaacaaggcccugccugcuccuaucgagaagaccaucagcaaggccaagggccagccuagggaaccucagguguacacacugccuccaagcagggacgagcugaccaagaaucaggugucccugaccugccucgugaagggcuucuacccuuccgauaucgccguggagugggagagcaacggccagccugagaacaacuacaagaccacuccuccugugcuggacagcgacggcucauucuuccuguacagcaagcugacaguggacaaguccagguggcagcagggcaacguguucagcugcagcgugcugcacgaagcccugcacagccacuacacccagaagucccugucucugagcccuggcaaaugauaauaggcuggagccucgguggccuagcuucuugccccuugggccuccccccagccccuccuccccuuccugcacccguacccccguggucuuugaauaaagucugagugggcggc

SEQ ID NO: 6 - RNA, 881 nt, Artificial Sequence
  Synthetic: Construct Sequence
gggaaauaagagagaaaagaagaguaagaagaaauauaagaccccggcgccgccaccauggaaacacccgcucagcugcuguuccugcugcugcuguggcugccugauaccacaggcgagaucgugcugacacagagcccuggcacacugucacugucuccaggcgaaagagccacacugagcuguagagccagccagagccugguguccagcuacuucggcugguaucagcagaagagaggccagucuccucggcugcugaucuacgccgcuucuacaagagccaccggcauucccgauagauucagcggcucuggcagcggcaccgauuucacccugacaaucagcagacuggaacccgaggacuucgccguguacuacugucagcaguacggcaacacacccuucaccuuuggcggaggcaccaagguggaaaucaagagaacaguggcugcucccagcguguucaucuucccaccuuccgacgagcagcugaagucuggcacagccucugucgugugccugcugaacaacuucuacccucgggaagccaaggugcaguggaagguggacaacgcccugcagagcggcaacagccaagagagcgugacagagcaggacagcaaggacuccaccuacagccugagcagcacacugacccugagcaaggccgacuacgagaagcacaagguguacgccugcgaagugacacaccagggccugucuagcccugugaccaagagcuucaacagaggcgagugcugauaauaggcuggagccucgguggccuagcuucuugccccuugggccuccccccagccccuccuccccuuccugcacccguacccccguggucuuugaauaaagucugagugggcggc

SEQ ID NO: 7-12 - skipped (6 entries)

SEQ ID NO: 13 - RNA, 57 nt, Artificial Sequence
  Synthetic: 5′ UTR Sequence
gggaaauaagagagaaaagaagaguaagaagaaauauaagaccccggcgccgccacc

SEQ ID NO: 14 - RNA, 119 nt, Artificial Sequence
  Synthetic: 3′ UTR Sequence
ugauaauaggcuggagccucgguggccuagcuucuugccccuugggccuccccccagccccuccuccccuuccugcacccguacccccguggucuuugaauaaagucugagugggcggc

SEQ ID NO: 15 - RNA, 47 nt, Artificial Sequence
  Synthetic: 5′ UTR v1.0
gggaaauaagagagaaaagaagaguaagaagaaauauaagagccacc

SEQ ID NO: 16 - RNA, 119 nt, Artificial Sequence
  Synthetic: 3′UTR v1.0
ugauaauaggcuggagccucgguggccaugcuucuugccccuugggccuccccccagccccuccuccccuuccugcacccguacccccguggucuuugaauaaagucugagugggcggc